(19) United States
(12) Patent Application Publication
(10) Pub. No.: US 2026/0327633 A1
(43) Pub. Date: Oct. 22, 2026

(54) CODON OPTIMIZATION AND METHODS OF USE THEREOF

(71) Applicant: General Medicines Inc., Philadelphia, PA (US)

(72) Inventor: Joshua Joyner SIMS, Villanova, PA (US)

(21) Appl. No.: 19/161,854

(22) PCT Filed: Mar. 6, 2024
(86) PCT No.: PCT/US2024/018742

§ 371 Date: Sep. 3, 2025

Related U.S. Application Data
(60) Provisional application No. 63/488,627, filed on Mar. 6, 2023.
(60) Provisional application No. 63/498,902, filed on Apr. 28, 2023.

(51) Int. Cl. C12N 15/67; A61K 48/00; C12N 15/86; G16B 20/50

(52) U.S. Cl. CPC C12N 15/67; A61K 48/0066; C12N 15/86; G16B 20/50; C12N 2750/14143; C12N 2800/22

(57) ABSTRACT

Featured are methods and applications of codon optimization of a gene product. In particular, the disclosure features methods of codon optimization by reducing the frequency of m6A modifications so as to promote increased mRNA half-life and stability for the purpose of enhancing protein production. Additional methods of delivering codon optimized gene products are disclosed. The methods of the disclosure are clinically relevant for gene and cellular therapies and vaccine development.

### SEQUENCE LISTING

The instant application contains a Sequence Listing which has been submitted electronically in XML format and is hereby incorporated by reference in its entirety. Said XML copy, created Mar. 1, 2024, is named “51772-004WO4_Sequence_Listing_3_1_24” and is 18,345 bytes in size.

### FIELD OF THE INVENTION

This disclosure relates to the field of nucleic acid engineering. Specifically, the present disclosure provides compositions and methods for modifying the codon sequence of a polypeptide-encoding nucleic acid so as to improve expression of the desired polypeptide in an organism of interest, such as a human.

### BACKGROUND

N(6)-methyladenosine (m6A), the most prevalent mRNA modification in mammals, influences wide-ranging aspects of gene expression in diverse physiological and pathophysiological processes. The METTL3-METTL14 methyltransferase complex installs m6A methylation on mRNA in a common DRACH sequence motif (D=A, G, or U; R=A or G; H=A, C, or U), but only a fraction of DRACH sequences (~5%) in a subset of cellular transcripts are selected for methylation. Additionally, m6A exhibits a marked regional bias in its transcriptomic distribution, being strongly enriched in unusually long internal exons and near stop codons. Despite the central importance of specific m6A deposition in m6A-mediated gene regulation, the mechanistic basis for m6A specificity has remained poorly understood. There remains a need for compositions and methods that leverage this mechanistic basis to improve transgene expression in an organism of interest.

### SUMMARY OF THE INVENTION

In a first aspect, the disclosure features a method of codon optimization of a polynucleotide sequence that encodes a polypeptide of interest. The method includes substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide.

In some embodiments, the polynucleotide sequence comprises a plurality of DRACH motifs, and wherein the substituting is performed only in a subset of the DRACH motifs. In some embodiments, the substituting is performed only in a DRACH motif that is separated from a splice junction site within the polynucleotide sequence by at least 10 nucleotides. In some embodiments, the substituting is performed only in a DRACH motif that is separated from a splice junction site within the polynucleotide sequence by at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 110 nucleotides, at least 120 nucleotides, at least 130 nucleotides, at least 140 nucleotides, at least 150 nucleotides, at least 160 nucleotides, at least 170 nucleotides, at least 180 nucleotides, at least 190 nucleotides, at least 200 nucleotides, or more.

In some embodiments, the substituting is performed only in a DRACH motif that is separated from a splice junction site within the polynucleotide sequence by from about 10 nucleotides to about 200 nucleotides, by from about 20 nucleotides to about 190 nucleotides, by from about 30 nucleotides to about 180 nucleotides, by from about 40 nucleotides to about 170 nucleotides, by from about 50 nucleotides to about 160 nucleotides, by from about 60 nucleotides to about 150 nucleotides, by from about 70 nucleotides to about 140 nucleotides, by from about 80 nucleotides to about 130 nucleotides, by from about 90 nucleotides to about 120 nucleotides, or by about 100 nucleotides.

In some embodiments, the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC. In some embodiments, the substituting is performed in a DRACH motif selected from the group consisting of AGACA, AGACT, GAACC, GAACT, GGACA, GGACT, and TGACT.

In some embodiments, the substituting of the one or more nucleotides in the DRACH motif is performed using one or more codon usage guidelines selected from the group consisting of:
(a) never using the codon GAC to encode aspartic acid when eliminating the DRACH motif;
(b) never using the codon GAA to encode glutamic acid when eliminating the DRACH motif;
(c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif; and/or
(f) always using the codon ACG to encode threonine when eliminating the DRACH motif.

In some embodiments, the codon optimization further comprises implementing, across the full polynucleotide sequence, one or more codon usage guidelines selected from the group consisting of:
(a) never using the codon GAC to encode aspartic acid across the full polynucleotide sequence;
(b) never using the codon GAA to encode glutamic acid across the full polynucleotide sequence;
(c) never using the codon GGA to encode glycine across the full polynucleotide sequence;
(d) never using the codon AAA to encode lysine across the full polynucleotide sequence;
(e) never using the codon AAC to encode asparagine across the full polynucleotide sequence; and/or
(f) always using the codon ACG to encode threonine across the full polynucleotide sequence.

In some embodiments, prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

In some embodiments, prior to the substituting of the one or more nucleotides in the DRACH motif, the polynucleotide sequence has been codon optimized by modulating polynucleotide guanine-cytosine (GC) content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, after the substituting of the one or more nucleotides in the DRACH motif, the method of codon optimization further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, after the substituting of the one or more nucleotides in the DRACH motif, the method of codon optimization further comprises substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, after the substituting of the one or more nucleotides in the DRACH motif, the method of codon optimization further comprises codon optimizing by way of a codon optimization webtool, codon optimization software, quantum computing, a heuristic scoring method, a neural network, or a combination thereof.

In some embodiments, after the substituting of the one or more nucleotides in the DRACH motif, the method of codon optimization further comprises modulating polynucleotide GC content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, the amino acid sequence of the protein or polypeptide shares at least 85% sequence identity to a wild-type amino acid sequence of the protein or polypeptide following codon optimization. In some embodiments, the amino acid sequence of the protein or polypeptide shares 100% sequence identity to a wild-type amino acid sequence of the protein or polypeptide following codon optimization.

In some embodiments, the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method. In some embodiments, the number of DRACH motifs in the polynucleotide sequence is reduced by 100%, as compared to a corresponding polynucleotide sequence that has not been subjected to the method.

In some embodiments, the method of codon optimization results in increased mRNA transcript stability or half-life.

In some embodiments, the increased mRNA transcript stability or half-life is assessed by an increase in concentration or relative abundance as compared to a corresponding polynucleotide sequence that has not been subjected to the method.

In some embodiments, the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

In some embodiments, the increased mRNA transcript stability or half-life is assessed by reduced rates of decay as detected by one or more pulse-chase methodologies.

In some embodiments, reducing the number of DRACH motifs in the polynucleotide sequence reduces the number of N(6)-methyladenosine (m6A) modifications in an mRNA transcript as compared to a corresponding polynucleotide sequence that has not been subjected to the method.

In some embodiments, the method of codon optimization results in increased protein expression or stability as compared to the protein encoded by a corresponding polynucleotide sequence that has not been subjected to the method. In some embodiments, the corresponding polynucleotide sequence is a wild-type polynucleotide sequence.

In some embodiments, the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a corresponding polynucleotide sequence that has not been subjected to the method.

In some embodiments, the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a corresponding polynucleotide sequence that has not been subjected to the method.

In some embodiments, the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

In another aspect, the disclosure features a polynucleotide produced by the method of any one of the foregoing aspects or embodiments of the disclosure.

In another aspect, the disclosure features a method of delivering a polypeptide-encoding polynucleotide sequence to a host cell, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the resulting polynucleotide sequence to the host cell.

In a further aspect, the disclosure features a method of expressing an mRNA transcript in a host cell from a polypeptide-encoding polynucleotide sequence, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the polynucleotide sequence to the host cell.

In yet another aspect, the disclosure features a method of expressing a protein in a host cell from a polynucleotide sequence encoding the protein, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded protein, and (ii) providing the polynucleotide sequence to the host cell.

In a further aspect, the disclosure features a method of delivering a polypeptide-encoding polynucleotide sequence to a subject, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the resulting polynucleotide sequence to the subject.

In a further aspect, the disclosure features a method of expressing an mRNA transcript in a subject from a polypeptide-encoding polynucleotide sequence, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the polynucleotide sequence to the subject.

In another aspect, the disclosure features a method of expressing a protein in a subject from a polynucleotide sequence encoding the protein, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded protein, and (ii) providing the polynucleotide sequence to the subject.

In some embodiments of any of the preceding aspects of the disclosure, the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC. In some embodiments, the substituting is performed in a DRACH motif is selected from the group consisting of AGACA, AGACT, GAACC, GAACT, GGACA, GGACT, and TGACT.

In some embodiments, the substituting of the one or more nucleotides in the DRACH motif is performed using one or more codon usage guidelines selected from the group consisting of:
(a) never using the codon GAC to encode aspartic acid when eliminating the DRACH motif;
(b) never using the codon GAA to encode glutamic acid when eliminating the DRACH motif;
(c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif; and/or
(f) always using the codon ACG to encode threonine when eliminating the DRACH motif.

In some embodiments, the codon optimization further comprises implementing, across the full polynucleotide sequence, one or more codon usage guidelines selected from the group consisting of:
(a) never using the codon GAC for aspartic acid across the full polynucleotide sequence;
(b) never using the codon GAA for glutamic acid across the full polynucleotide sequence;
(c) never using the codon GGA for glycine across the full polynucleotide sequence;
(d) never using the codon AAA for lysine across the full polynucleotide sequence;
(e) never using the codon AAC for asparagine across the full polynucleotide sequence; and/or
(f) always using the codon ACG for threonine across the full polynucleotide sequence.

In some embodiments, prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

In some embodiments, prior to the substituting of the one or more nucleotides in the DRACH motif, the polynucleotide sequence has been codon optimized by modulating polynucleotide GC content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, after the substituting of the one or more nucleotides in the DRACH motif, the method further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, after the substituting of the one or more nucleotides in the DRACH motif, the method further comprises substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, after the substituting of the one or more nucleotides in the DRACH motif, the method further comprises codon optimizing by way of a codon optimization webtool, codon optimization software, quantum computing, a heuristic scoring method, a neural network, or a combination thereof.

In some embodiments, after the substituting of the one or more nucleotides in the DRACH motif, the method of codon optimization further comprises modulating polynucleotide GC content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, the amino acid sequence of the protein or polypeptide shares at least 85% sequence identity to a wild-type amino acid sequence of the protein or polypeptide following codon optimization.

In some embodiments, the amino acid sequence of the protein or polypeptide shares 100% sequence identity to a wild-type amino acid sequence of the protein or polypeptide following codon optimization.

In some embodiments, the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method.

In some embodiments, the number of DRACH motifs in the polynucleotide sequence is reduced by 100%, as compared to a corresponding polynucleotide sequence that has not been subjected to the method.

In some embodiments, the method results in increased mRNA transcript stability or half-life.

In some embodiments, the increased mRNA transcript stability or half-life is assessed by an increase in concentration or relative abundance as compared to a corresponding polynucleotide sequence that has not been subjected to the method.

In some embodiments, the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

In some embodiments, the increased mRNA transcript stability or half-life is assessed by reduced rates of decay as detected by one or more pulse-chase methodologies.

In some embodiments, reducing the number of DRACH motifs in the polynucleotide sequence reduces the number of N(6)-methyladenosine (m6A) modifications in an mRNA transcript as compared to a corresponding polynucleotide sequence that has not been subjected to the method.

In some embodiments, the method results in increased protein expression or stability as compared to the protein encoded by a corresponding polynucleotide sequence that has not been subjected to the method.

In some embodiments, the corresponding polynucleotide sequence is a wild-type polynucleotide sequence.

In some embodiments, the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a corresponding polynucleotide sequence that has not been subjected to the method.

In some embodiments, the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a corresponding polynucleotide sequence that has not been subjected to the method.

In some embodiments, the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

In some embodiments, the polynucleotide is delivered to the host cell by contacting the host cell with a vehicle comprising the polynucleotide.

In some embodiments, the polynucleotide is delivered to the subject by administering to the subject a vehicle comprising the polynucleotide.

In some embodiments, the vehicle is selected from the group consisting of a lipid nanoparticle, a liposome, a viral genome, and a viral vector.

In some embodiments, the viral vector is an adeno-associated virus (AAV), an adenovirus, a retrovirus, or a lentivirus.

In some embodiments, the viral vector is an AAV. In some embodiments, the AAV is pseudotyped. In some embodiments, the AAV comprises an inverted terminal repeat (ITR) sequence and a capsid protein derived from different serotypes.

In some embodiments, the delivery to the host cell occurs in vivo, in vitro, or ex vivo.

In some embodiments, the delivery to the subject occurs by way of (i) in vivo administration of the polynucleotide to the subject or (ii) ex vivo expression of the polynucleotide in a host cell, followed by administration of the host cell to the subject.

In some embodiments, the method is used to treat a subject that has or is at risk of having a disease characterized by low expression or activity of a protein.

In another aspect, the disclosure features a method of treating a subject who has or is at risk of developing a disease, the method comprising (i) substituting one or more nucleotides in a DRACH motif within a polynucleotide sequence encoding a polypeptide associated with the disease with an equivalent quantity of alternative nucleotides that eliminate the DRACH motif, wherein the substituting does not alter the amino acid sequence of the encoded polypeptide, and (ii) administering the polynucleotide sequence to the subject.

In a further aspect, the disclosure features a method of treating a subject who has or is at risk of developing a disease, the method comprising administering a polynucleotide sequence encoding a polypeptide associated with the disease to the subject, wherein prior to the administering, one or more nucleotides in a DRACH motif within the polynucleotide sequence has been substituted with an equivalent quantity of alternative nucleotides that eliminate the DRACH motif, wherein the substituting does not alter the amino acid sequence of the encoded polypeptide.

In some embodiments, the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC. In some embodiments, the substituting is performed in a DRACH motif selected from the group consisting of AGACA, AGACT, GAACC, GAACT, GGACA, GGACT, and TGACT.

In some embodiments, the substituting of the one or more nucleotides in the DRACH motif is performed using one or more codon usage guidelines selected from the group consisting of: (a) never using the codon GAC to encode aspartic acid when eliminating the DRACH motif;
(b) never using the codon GAA to encode glutamic acid when eliminating the DRACH motif; (c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif; and/or (f) always using the codon ACG to encode threonine when eliminating the DRACH motif.

In some embodiments, prior to the administering, the polynucleotide sequence has been codon optimized by a method comprising implementing, across the full polynucleotide sequence, one or more codon usage guidelines selected from the group consisting of:
(a) never using the codon GAC to encode aspartic acid across the full polynucleotide sequence;
(b) never using the codon GAA to encode glutamic acid across the full polynucleotide sequence;
(c) never using the codon GGA to encode glycine across the full polynucleotide sequence;
(d) never using the codon AAA to encode lysine across the full polynucleotide sequence;
(e) never using the codon AAC to encode asparagine across the full polynucleotide sequence; and/or
(f) always using the codon ACG to encode threonine across the full polynucleotide sequence.

In some embodiments, prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

In some embodiments, prior to the substituting of the one or more nucleotides in the DRACH motif, the polynucleotide sequence has been codon optimized by modulating polynucleotide guanine-cytosine (GC) content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, after the substituting of the one or more nucleotides in the DRACH motif, the method further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

In some embodiments, after the substituting of the one or more nucleotides in the DRACH motif, the method further comprises substituting one or more codons in the polynucleotide sequence with an equivalent quantity of synonymous codons that occur naturally at a higher frequency in a target organism relative to the frequency with which the one or more unmodified codons occur in the target organism. In some embodiments, the target organism is a mammal, optionally wherein the mammal is a human.

In some embodiments, after the substituting of the one or more nucleotides in the DRACH motif, the method further comprises codon optimizing by way of a codon optimization webtool, codon optimization software, quantum computing, a heuristic scoring method, a neural network, or a combination thereof.

In some embodiments, after the substituting of the one or more nucleotides in the DRACH motif, the method further comprises modulating the polynucleotide GC content, polynucleotide secondary structure, mRNA motifs, ribosomal binding sites, and/or rates of translation.

In some embodiments, the polynucleotide GC content is adjusted to between 50% and 80%. In some embodiments, the polynucleotide GC content is adjusted to between 65% and 75%.

In some embodiments, the amino acid sequence of the protein or polypeptide shares at least 85% sequence identity to a wild-type amino acid sequence of the protein or polypeptide following codon optimization.

In some embodiments, the amino acid sequence of the protein or polypeptide shares 100% sequence identity to a wild-type amino acid sequence of the protein or polypeptide following codon optimization.

In some embodiments, the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method.

In some embodiments, the number of DRACH motifs in the polynucleotide sequence is reduced by 100%, as compared to a corresponding polynucleotide sequence that has not been subjected to the method.

In some embodiments, the method results in increased mRNA transcript stability or half-life.

In some embodiments, the increased mRNA transcript stability or half-life is assessed by an increase in concentration or relative abundance as compared to a reference.

In some embodiments, the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

In some embodiments, reducing the number of DRACH motifs in the polynucleotide sequence reduces the number of N(6)-methyladenosine (m6A) modifications in an mRNA transcript as compared to a corresponding polynucleotide sequence that has not been subjected to the method.

In some embodiments, the method results in increased protein expression or stability as compared to a reference.

In some embodiments, the corresponding polynucleotide sequence is a wild-type polynucleotide sequence.

In some embodiments, the reference is a sample obtained from a subject that has not received treatment or a sample obtained from the subject prior to treatment.

In some embodiments, the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a reference.

In some embodiments, the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a reference.

In some embodiments, the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

In some embodiments, the polynucleotide is administered to the subject by contacting the subject with a vehicle comprising the polynucleotide.

In some embodiments, the vehicle is selected from a lipid nanoparticle, a liposome, a viral genome, and a viral vector. In some embodiments, the viral vector is an adeno-associated virus (AAV), an adenovirus, a retrovirus, a lentivirus, or a double-stranded DNA virus. In some embodiments, the viral vector is an AAV. In some embodiments, the AAV is pseudotyped. In some embodiments, the AAV comprises an inverted terminal repeat (ITR) sequence and a capsid protein derived from different serotypes.

In some embodiments, the administration occurs by directly providing the subject, in vivo, with the polynucleotide or by expressing the polynucleotide in a host cell ex vivo and subsequently administering the host cell to the subject.

In some embodiments, the delivery is performed by intramuscular, subcutaneous, intradermal, intravenous, intraperitoneal, intraarterial, intraventricular, intranasal, intraorbital, intracranial, or intraosseous administration.

In some embodiments, the treatment is a monotherapy. In some embodiments, the method is combined with the administration of one or more additional therapeutic agents.

In a further aspect, the disclosure features a kit comprising a package insert that instructs a user to perform a method of codon optimization that comprises modifying a polypeptide-encoding polynucleotide sequence by substituting one or more nucleotides in a DRACH motif in the polynucleotide sequence without altering the amino acid sequence of the encoded polypeptide, thereby eliminating the one or more DRACH motifs.

In a further aspect, the disclosure features a kit comprising a package insert that instructs a user to perform the method of any one of the above aspects or embodiments of the disclosure.

In some embodiments, the kit comprises a device for administering the polynucleotide sequence to a subject. In some embodiments, the kit comprises one or more binding molecules to detect the expression of an mRNA transcript encoding the polypeptide or the expression or activity of the polypeptide.

### BRIEF DESCRIPTION OF THE DRAWINGS

The accompanying drawings are included to illustrate embodiments of the disclosure and further an understanding of its implementations.

FIG. 1 is a schematic showing the features of the pAAV ZsGreen1 plasmid map.

FIG. 2A is a bar graph showing relative ZsGreen fluorescent protein production from all-best codon optimized open reading frames (ORFs) with or without removal of DRACH motifs. Flow cytometry measurements of HEK293T cells were taken 2 or 3 days following a 1:4 transfection with plasmids containing the all-best codon optimized ORFs, in which all codons were substituted with synonymous codons with the highest usage frequency according to Table 1. “+m6A” denotes that 14 DRACH motifs were present in the transfected ORF, and sample groups labeled “−m6A” were further modified with the six codon usage rules to eliminate all present DRACH motifs in the coding sequence prior to transfection.

FIG. 2B is a bar graph showing relative ZsGreen fluorescent protein production from all-best codon optimized ORFs with or without removal of DRACH motifs. Flow cytometry measurements of HEK293T cells were taken 2 days following a 1:16 transfection with plasmids containing the all-best codon optimized ORFs, in which all codons were substituted with synonymous codons with the highest usage frequency according to Table 1. “+m6A” denotes that 14 DRACH motifs were present in the transfected ORF, and “−m6A” denotes that the ORFs were further modified with the six codon usage rules to eliminate all present DRACH motifs in the coding sequence prior to transfection.

FIG. 3A is a bar graph showing relative ZsGreen fluorescent protein production from proportional usage codon optimized ORFs with or without removal of DRACH motifs. Flow cytometry measurements of HEK293T cells were taken 2 or 3 days following a 1:4 transfection with plasmids containing the proportional usage codon optimized ORFs, in which codons were represented based on their reported frequency shown in Table 1. “+m6A” denotes that 16 DRACH motifs were present in the transfected ORF, and “−m6A” denotes that the ORFs were further modified with the six codon usage rules to eliminate all present DRACH motifs in the coding sequence prior to transfection.

FIG. 3B is a bar graph showing relative ZsGreen fluorescent protein production from proportional usage codon optimized ORFs with or without removal of DRACH motifs. Flow cytometry measurements of HEK293T cells were taken 2 days following a 1:16 transfection with plasmids containing the proportional usage codon optimized ORFs, in which codons were represented based on their reported frequency shown in Table 1. “+m6A” denotes that 16 DRACH motifs present in the transfected ORF, and “−m6A” denotes that the ORFs were further modified with the six codon usage rules to eliminate all present DRACH motifs in the coding sequence prior to transfection.

FIG. 4 is a bar graph showing relative GLP-1-Fc protein secretion from ORFs that were codon optimized using a commercially available codon optimization webtool, with or without further modifications to remove DRACH motifs. ORFs in which DRACH motifs were removed is denoted “−m6A.” Protein secretion was measured via enzyme-linked immunosorbent assay (ELISA).

FIG. 5 is a bar graph showing relative GLP-1-Fc protein secretion as measured by from HEK293T cells transfected with AAVs containing ORFs that were codon optimized using four distinct strategies. “CH” denotes that the GLP-1-Fc ORF was codon optimized by replacing every codon with a synonymous codon having the highest usage. “IDT” denotes that the GLP-1-Fc ORF was codon optimized using a commercially available codon optimization webtool. “−m6A” further denotes that following the indicated codon optimization strategy, the ORFs were further modified to remove DRACH sequences. Protein concentrations were measured by ELISA.

FIG. 6A is a graph showing the detectable serum GLP-1-Fc protein levels in mice at different days following a low-dose (1×10⁹ genome copies per mouse) intramuscular administration of AAVs containing the native ORF or an ORF that was codon optimized using one of four distinct strategies. “CH” denotes that the GLP-1-Fc ORF was codon optimized by replacing every codon with a synonymous codon having the highest usage. “IDT” denotes that the GLP-1-Fc ORF was codon optimized using a commercially available codon optimization webtool. “m6A” denotes that following the indicated codon optimization strategy, the ORFs were further modified to remove DRACH sequences. Protein concentrations were measured by ELISA.

FIG. 6B is a graph showing the detectable serum GLP-1-Fc protein levels in mice at different days following a high-dose (1×10¹⁰ genome copies per mouse) intramuscular administration of AAVs containing the native ORF or an ORF that was codon optimized using one of four distinct strategies. “CH” denotes that the GLP-1-Fc ORF was codon optimized by replacing every codon with a synonymous codon having the highest usage. “IDT” denotes that the GLP-1-Fc ORF was codon optimized using a commercially available codon optimization webtool. “m6A” further denotes that following the indicated codon optimization strategy, the ORFs were further modified to remove DRACH sequences. Protein concentrations were measured by ELISA.

FIG. 7 is a bar graph showing the detectable serum GLP-1-Fc protein levels in mice 14 days after receiving a low-dose (1×10⁹ genome copies per mouse) intramuscular administration of AAVs containing native ORF or an ORF that was codon optimized using one of two distinct codon optimization strategies. “CH” denotes that the GLP-1-Fc ORF was codon optimized by replacing every codon with a synonymous codon having the highest usage. “m6A” denotes that following codon optimization, the ORFs were further modified to remove DRACH sequences. Protein concentrations were measured by ELISA.

FIG. 8A is a bar graph showing the detectable serum GLP-1-Fc protein levels in mice 28 days after receiving a low-dose (1×10⁹ genome copies per mouse) intramuscular administration of AAVs containing ORFs that were codon optimized using a commercially available codon optimization webtool in which DRACH motifs were left unmodified (+m6A) or were removed by further substitution (−m6A). Protein concentrations were measured by ELISA.

FIG. 8B is a bar graph showing the detectable serum GLP-1-Fc protein levels in mice 28 days after receiving a low-dose (1×10⁹ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Protein concentrations were measured by ELISA.

FIG. 8C is a bar graph showing the detectable serum GLP-1-Fc protein levels in mice 28 days after receiving a high-dose (1×10¹⁰ genome copies per mouse) intramuscular administration of AAVs containing ORFs that were codon optimized using a commercially available codon optimization webtool in which DRACH motifs were left unmodified (+m6A) or were removed by further substitution (−m6A). Protein concentrations were measured by ELISA.

FIG. 8D is a bar graph showing the detectable serum GLP-1-Fc protein levels in mice 28 days after receiving a high-dose (1×10¹⁰ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Protein concentrations were measured by ELISA.

FIG. 9A is a bar graph showing relative GLP-1-Fc mRNA transcript levels in mouse muscle tissue 60 days after receiving a low-dose (1×10⁹ genome copies per mouse) intramuscular administration of AAVs containing ORFs that were codon optimized using a commercially available codon optimization webtool in which DRACH motifs were left unmodified (+m6A) or were removed by further substitution (−m6A). Transcript levels were measured by qPCR.

FIG. 9B is a bar graph showing relative GLP-1-Fc mRNA transcript levels in mouse muscle tissue 60 days after receiving a low-dose (1×10⁹ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Transcript levels were measured by qPCR.

FIG. 9C is a bar graph showing relative GLP-1-Fc mRNA transcript levels in mouse muscle tissue 60 days after receiving a high-dose (1×10¹⁰ genome copies per mouse) intramuscular administration of AAVs containing ORFs that were codon optimized using a commercially available codon optimization webtool in which DRACH motifs were left unmodified (+m6A) or were removed by further substitution (−m6A). Transcript levels were measured by qPCR.

FIG. 9D is a bar graph showing relative GLP-1-Fc mRNA transcript levels in mouse muscle tissue 60 days after receiving a high-dose (1×10¹⁰ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Transcript levels were measured by qPCR.

FIG. 10A is a bar graph showing relative GLP-1-Fc mRNA transcript levels in mouse liver tissue 60 days after receiving a low-dose (1×10⁹ genome copies per mouse) intramuscular administration of AAVs containing ORFs that were codon optimized using a commercially available codon optimization webtool in which DRACH motifs were left unmodified (+m6A) or were removed by further substitution (−m6A). Transcript levels were measured by qPCR.

FIG. 10B is a bar graph showing relative GLP-1-Fc mRNA transcript levels in mouse liver tissue 60 days after receiving a low-dose (1×10⁹ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Transcript levels were measured by qPCR.

FIG. 10C is a bar graph showing relative GLP-1-Fc mRNA transcript levels in mouse liver tissue 60 days after receiving a high-dose (1×10¹⁰ genome copies per mouse) intramuscular administration of AAVs containing ORFs that were codon optimized using a commercially available codon optimization webtool in which DRACH motifs were left unmodified (+m6A) or were removed by further substitution (−m6A). Transcript levels were measured by qPCR.

FIG. 10D is a bar graph showing relative GLP-1-Fc mRNA transcript levels in mouse liver tissue 60 days after receiving a high-dose (1×10¹⁰ genome copies per mouse) intramuscular administration of AAVs containing ORFs that were codon optimized by replacing every codon with a synonymous codon having the highest usage, in which DRACH motifs were left unmodified (+m6A) or were removed by further substitution (−m6A). Transcript levels were measured by qPCR.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context (except where such number would exceed 100% of a possible value).

As used herein, the term “codon optimization” refers to a process of changing codons of a given gene or coding sequence in such a manner that is intended to increase expression of the encoded polypeptide, without altering the amino acid sequence of the polypeptide. This process utilizes the redundancy of the genetic code in which multiple three-base pair codon combinations encode a single amino acid. These codons, which differ in nucleic acid sequence but encode the same amino acid, are referred to herein as “synonymous codons.” As a non-limiting example, the amino acid leucine may be encoded by any one of the following six codons: UUA, UUG, CUA, CUG, CUU, or CUC, all of which are synonymous with each other. The process of codon optimization generally improves the efficacy of mRNA expression, mRNA stability, and/or polypeptide synthesis as compared to the unmodified gene or coding sequence. Codon optimization often improves expression of a target gene by modifying a codon sequence in a way that accounts for tRNA abundance in a cell type or organism, secondary structural elements in the polynucleotide sequence, and/or binding interactions of the polynucleotide sequence and one or more binding partners (e.g., ribosomes, methyltransferases, tRNA molecules, and other binding partners known in the art). An “alternative nucleotide” or “alternative codon” in reference to codon optimization refers to a different nucleotide or codon that ultimately encodes the same amino acid sequence of a polypeptide or a protein of interest.

As used herein, the terms “codon usage table,” “codon bias table,” “codon frequency lookup table,” and the like are used interchangeably to refer to a table that correlates each codon that may be used to encode a particular amino acid with the frequency with which that codon is used to encode its corresponding amino acid in a particular setting. The setting may be, for example, (i) a specific organism (e.g., a mammalian subject, such as a human), (ii) a specified class of genes within a given organism, or (iii) one or more synthetic polynucleotides. A hybrid codon usage table or hybrid codon bias table can also be constructed by combining two or more codon usage tables according to a variety of possible rules.

As used herein, an “organism” or a “target organism,” in reference to codon optimization refers to the organism intended to receive delivery of one or more polynucleotides (e.g., one or more codon optimized polynucleotides) described herein.

As used herein, a “coding sequence” refers to an open reading frame (ORF) in a nucleic acid that, upon expression, yields a polypeptide or protein. An ORF is a continuous stretch of DNA or RNA beginning with a start codon (e.g., methionine (ATG in the context of a DNA sequence or AUG in the context of an RNA sequence)) and ending with a stop codon (e.g., TAA, TAG or TGA in the context of a DNA sequence, or UAA, UAG or UGA in the context of an RNA sequence). An ORF typically encodes a protein. It will be understood that the sequences disclosed herein may further include additional elements, e.g., 5′ and 3′ untranslated regions (UTRs), but that those elements, unlike the ORF, need not necessarily be present in an RNA polynucleotide (e.g., an mRNA transcript) disclosed herein.

As used herein in the context of a target protein product, the terms “level of expression” or “expression level” are used interchangeably and refer to the amount of the protein product in a subject or biological sample. “Expression” of a desired protein, as the term is used herein, refers to the overall process by which a gene of interest is processed (e.g., in a subject, in a cell, or ex vivo by way of one or more enzymes or organelles obtained from a cell) so as to produce the protein product. Specifically, the term “expression” may be used to describe one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Accordingly, genes that are expressed using the compositions and methods described herein include those that are transcribed into a polynucleotide (such as mRNA) and then translated into a polypeptide or protein, as well as those genes that are transcribed into an RNA polynucleotide but that are not translated into a polypeptide (for example, transfer and ribosomal RNAs). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis.

Expression of a gene of interest in a subject can manifest, for example, by detecting: an increase in the quantity or concentration of mRNA encoding a corresponding protein (as assessed, e.g., using RNA detection procedures described herein or known in the art, such as quantitative polymerase chain reaction (qPCR), RT-PCR, and RNA seq techniques), an increase in the quantity or concentration of a corresponding protein (as assessed, e.g., using protein detection methods described herein or known in the art, such as enzyme-linked immunosorbent assays (ELISA), mass spectrometry, Western blot analysis, flow cytometry, immunofluorescence, colorimetry assays, or an array using targeting antibodies or hybridizing nucleotides, among others), and/or an increase in the activity of a corresponding protein (e.g., in the case of an enzyme, as assessed using an enzymatic activity assay known in the art) in a sample obtained from the subject.

As used herein, the terms “polynucleotide” or “nucleic acid” refer to polymers of nucleotides of any length and include DNA and/or RNA. The nucleotides may be deoxyribonucleotides, ribonucleotides, modified nucleotides (e.g., containing modified nucleobases), and/or a nucleotide analog that can be incorporated into a polymer by DNA or RNA polymerase or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or different molecules, the regions may include all of one or more of the molecules, but more typically involve only a region of some of the molecules. One of the molecules of a triple-helical region often is an oligonucleotide.

The term “polynucleotide” specifically includes complementary DNA (cDNA). In some embodiments, a polynucleotide is a codon optimized gene product, in which a polynucleotide (e.g., a polynucleotide encoding a protein of interest) is subjected to one or more methods of codon optimization described herein.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleoside modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-Omethyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, the terms “adenine” and “adenosine” are interchangeable terms in reference to a nucleotide that has an adenine base. As used herein, the terms “cytosine” and “cytidine” are interchangeable terms in reference to a nucleotide that has a cytosine base. As used herein, the terms “guanine” and “guanidine” are interchangeable terms in reference to a nucleotide that has a guanine base. As used herein, the terms “thymine” and “thymidine” are interchangeable terms in reference to a nucleotide that has a thymine base. As used herein, the term “uracil” and “uridine” are interchangeable terms in reference to a nucleotide that has a uracil base.

As used herein, the term “splice junction site” refers to a region in a nucleic acid (e.g., DNA or RNA) sequence that is the boundary between an intron and an exon in a gene. Features of splice junction sites or “splice sites” as well as mechanisms of splicing are described in the art, such as Roca et al. Genes Dev. 27 (2): 129-144, 2013, which is hereby incorporated by reference.

As used herein, “messenger RNA,” “mRNA,” or “mRNA transcript” is any RNA molecule that encodes a (at least one) polypeptide or fragment thereof and can be translated to produce the encoded polypeptide or the fragment thereof in vitro, in vivo, in situ, or ex vivo. Structural and topological features as well as post-transcriptional modifications of mRNA are described herein and are well-known in the art.

As used herein, the term “peptide” refers to a polymer containing a plurality of amino acid monomers (or analogs thereof) and that is less than or equal to 50 amino acids in length (e.g., about 5, 10, 15, 20, 25, 30, 35, 40, 45, or 50 amino acids in length).

As used herein, the term “polypeptide” refers to a polymer of amino acid residues linked together by peptide bonds. The term, as used herein, encompasses polypeptides of any size, structure, or function. Thus, polypeptides include gene products, naturally occurring polypeptides, synthetic polypeptides, homologs, orthologs, paralogs, fragments, and other equivalents, variants, and analogs of the foregoing. A polypeptide may be a single molecule or may be a multi-molecular complex such as a dimer, a trimer, or a tetramer. The term polypeptide may also apply to amino acid polymers in which one or more amino acid residues are an artificial chemical analog of a corresponding naturally occurring amino acid.

As used herein, the term “protein” refers to contiguous amino acids or amino acid residues. Typically, proteins have a function. However, proteins also encompass polypeptides and smaller contiguous amino acid sequences that do not have a functional activity. Examples of functional proteins include, but are not limited to, enzymes (such as dehydrogenases, hydrolases, oxidoreductases, transferases, lyases, and ligases, among others), receptors, receptor ligands, cytokines, antibodies, immunomodulatory molecules, signaling molecules, or proteins that are tagged or modified (e.g., for diagnostic or other clinical applications). Useful general classes of enzymes include, but are not limited to, proteases, cellulases, lipases, hemicellulases, laccases, amylases, glucoamylases, esterases, lactases, polygalacturonases, galactosidases, ligninases, oxidases, peroxidases, glucose isomerases, nitrilases, hydroxylases, polymerases and depolymerases. In addition to enzymes, the encoded proteins which can be used in this invention include, but are not limited to, transcription factors, antibodies, receptors, growth factors (any of the PDGFs, EGFs, FGFs, SCF, HGF, TGFs, TNFs, insulin, IGFs, LIFs, oncostatins, and CSFs), immunomodulators, peptide hormones, cytokines, integrins, interleukins, adhesion molecules, thrombomodulatory molecules, protease inhibitors, angiostatins, defensins, cluster of differentiation antigens, interferons, chemokines, antigens including those from infectious viruses and organisms, oncogene products, thrombopoietin, erythropoietin, tissue plasminogen activator, and any other biologically active protein which is desired for use in a clinical setting. Also included are deletion mutants of such proteins, individual domains of such proteins, fusion proteins made from such proteins, and mixtures of such proteins; particularly useful are those which have increased half-lives and/or increased activity.

As used herein, the term “DRACH motif” refers to a short, five base pair nucleic acid consensus sequence, in which the “D” in the D-R-A-C-H consensus sequence designates guanine, adenine, or uracil; “R” designates guanine or adenine; “A” designates adenine; “C” designates cytosine; and “H” designates adenine, uracil, or cytosine. A DRACH motif may be present in a polynucleotide sequence such as an RNA molecule (e.g., an mRNA transcript). As described herein, the presence of one or more DRACH motifs within a polynucleotide sequence can reduce the half-life of a polynucleotide (e.g., an mRNA transcript) and, in turn, reduce the concentration of a translated protein or polypeptide encoded by the polynucleotide. Without being bound by any particular theory, a DRACH motif may exhibit a higher likelihood of being modified by way of an N(6)-methyladenosine (“m6A”) modification on the adenine base in the third position of the motif. Such modifications may reduce the half-life of the modified mRNA transcript in a cell or sample that contains the mRNA transcript. One or more DRACH motifs in a nucleic sequence (e.g., a polynucleotide sequence; e.g., an mRNA sequence) may be removed or eliminated by substituting one or more nucleotides in the one or more DRACH motifs so as to furnish a synonymous codon that replaces a codon that formed part of the DRACH motif, while preserving the amino acid sequence of the encoded polypeptide product. The terms “removed” or “eliminated” and variations thereof in reference to a DRACH motif are understood to be interchangeable.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)₂ fragments) that are capable of specifically binding to a target protein. Fab and F(ab′)₂ fragments lack the Fc fragment of an intact antibody.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature 341:544-546, 1989) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “primer” refers to an oligonucleotide, either natural or synthetic, that is capable of forming a duplex with a polynucleotide template and then acting as a point of initiation of nucleic acid synthesis for extension from its 3′ end along the template nucleic acid so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, from 5 to 24 nucleotides, or from 14 to 36 nucleotides. In certain aspects, primers are universal primers or non-universal primers. Pairs of primers can flank a sequence of interest or a set of sequences of interest. Primers and probes can be degenerate in sequence. In certain aspects, primers bind adjacent to the target sequence, whether it is the sequence to be captured for analysis, or a tag that it to be copied.

As used herein, the term “vector” includes a nucleic acid, e.g., DNA (such as a plasmid) or RNA, that contains a polynucleotide encoding a gene product of interest, optionally in combination with one or more additional elements that facilitate expression of the gene product (such as a promoter, enhancer, untranslated region, or splicing modulator, among others). Vectors include both viral vectors as well as non-viral vectors. A variety of vectors have been developed for the delivery of polynucleotides encoding exogenous proteins into a eukaryotic or prokaryotic cell. Examples of such expression vectors are disclosed in, e.g., WO 1994/011026, the disclosure of which is incorporated herein by reference as it pertains to vectors suitable for the expression of a gene of interest. Certain vectors that can be used for the expression of transgenes as described herein include plasmids that contain regulatory sequences, such as promoter and enhancer regions, which direct gene transcription. Other useful vectors for expression of a transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site, which direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As used herein, the term “isolated” refers to a substance or entity that is altered or removed from the natural state (e.g., altered or removed from at least some component with which it is associated in the natural state). For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant (e.g., incorporated into a vector). Methods of isolating polynucleotides and proteins or polypeptides are routine in the art.

As used herein, the terms “naturally occurring” or “wild-type” means existing in nature without artificial aid or human involvement. “Naturally occurring” or “wild-type” may refer to a native form of a biomolecule, sequence, or entity.

As used herein, the phrase “operably linked” refers to a functional connection between two or more molecules, constructs, transcripts, entities, moieties, or the like. For example, one segment of DNA may be operably linked to another segment of DNA if they are positioned relative to one another on the same contiguous DNA molecule and have a structural or functional relationship, such as a promoter or enhancer that is positioned relative to a coding region so as to facilitate transcription of the coding region. In other examples, the operably linked nucleic acids are not contiguous, but are positioned in such a way that they have a functional relationship with each other as nucleic acids or as proteins that are expressed by them. Enhancers, for example, do not have to be contiguous. Linking may be accomplished by ligation at convenient restriction sites or by using synthetic oligonucleotide adaptors or linkers.

As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

As used herein, the term “stability” in reference to biological material or a molecule (e.g., a polynucleotide or a polypeptide) refers to the balance of production (e.g., transcription or translation) and decay or degradation or the steady-state levels of the biological material in a system, such as a whole organism, an organ, a tissue or subset of tissues, a cell or a subset of cells, or in a dish or receptacle. In some embodiments, stability refers to the half-life of the biological material or molecule.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur to a component of an organism (e.g., a tissue, a cell, or a subcellular fraction) when it is taken from the natural environment (e.g., from the body or natural structure) and placed into an artificial environment (e.g., a test tube or a culture dish, flask, or other receptacle) for experimental or clinical applications. In some instances, ex vivo experimentation or applications may involve administering (e.g., implanting, injecting, depositing, infusing, among other suitable routes of administration) the component to the same subject or a separate recipient subject following one or more ex vivo applications.

As used herein “modified” refers to a changed state or structure of a molecule (e.g., a polynucleotide; e.g., DNA or mRNA; e.g., a polypeptide or protein; e.g., an amino acid residue) of the invention. Molecules may be modified in many ways, such as structural modifications (e.g., mutation of one or more base pairs or amino acid residues) or chemical modifications (e.g., methylation, acetylation, reduction or oxidation, glycosylation, lipidation, ubiquitination, of one or more base pairs or amino acid residues). In some embodiments a molecule such as a DNA or an mRNA is modified to remove, reduce, or eliminate DRACH motifs to reduce the number of m6A methylation modifications in a gene or coding sequence of interest.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “identity” or “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be homologous to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be homologous to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term homologous necessarily refers to a comparison between at least two sequences (polynucleotide or polypeptide sequences).

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

In certain embodiments, the term “reference level” herein refers to a value from a “reference sample” to determine the effect induced by the methods described herein. A reference level may be a metric or measurement determined prior to administration or implementation of the method (e.g., one or more codon optimization methods or one or more methods of treatment described herein). A reference level may be a metric or measurement determined in a reference sample in which the methods described herein were not administered (e.g., a negative control sample; e.g., a healthy subject control or a subject with a disease or condition). A reference level may be a metric or measurement determined in a reference sample that exhibits a known or expected effect in order to evaluate the efficacy of the methods described herein (e.g., effects produced by routine methods of protein production or effects produced by known methods of treatment). In some embodiments, a reference level may be a predetermined value or a value. As the skilled artisan will appreciate, the reference level is predetermined and set to meet the requirements in terms of, for example, specificity and/or sensitivity. It may be, for example, that assay sensitivity or specificity, respectively, has to be set to certain limits, e.g., 80%, 90% or 95%. These requirements may also be defined in terms of positive or negative predictive values. In one embodiment, the reference level is determined in healthy individuals. The reference value in one embodiment has been predetermined in the disease entity to which a subject belongs. In certain embodiments, the reference level can be set to any percentage between, e.g., 25% and 75% of the overall distribution of the values in a disease entity investigated. In other embodiments, the reference level can be set to, for example, the median, tertiles, quartiles, or quintiles as determined from the overall distribution of the values in a disease entity investigated or in a given population. In one embodiment, the reference level is set to the median value as determined from the overall distribution of the values in a disease entity investigated. In some embodiments, the reference level may depend on the sex of the patient, e.g., males may have a different reference level than females.

As used herein, the term “sample” refers to a subset of its tissues, cells or component parts (e.g. body fluids, including but not limited to peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecules.

As used herein, “treatment” and “treating,” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the terms “effective amount,” “therapeutically effective amount,” and a “sufficient amount” of a composition described herein refer to a quantity sufficient to, when administered to the subject (e.g., a mammal; e.g., a human subject) yield beneficial or desired results, including clinical results, and, as such, an “effective amount” or synonym thereto depends upon the context in which it is being applied. The amount of a given composition described herein that will correspond to such an amount will vary depending upon an assortment of factors, such as the given therapeutic agent (e.g., polynucleotide, transgene, or coding sequence), the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition of the present disclosure is an amount which results in a beneficial or desired result in a subject as compared to a reference (e.g., the subject prior to treatment, a healthy control, or an untreated subject). As defined herein, a therapeutically effective amount of a composition of the present disclosure may be readily determined by one of ordinary skill by routine methods known in the art. A dosing regimen may be adjusted to provide the optimum therapeutic response.

As used herein, “administration” refers to dispensing, delivering, or applying a composition of the disclosure to a subject by any suitable route for delivery of the composition (e.g., a polynucleotide; e.g., a polynucleotide encoding a transgene), to the desired location in the subject. Exemplary routes of administration include intramuscular, subcutaneous, intradermal, intravenous, intraperitoneal, intraarterial, intraventricular, intranasal, intraorbital, intracranial, or intraosseous administration.

As used herein, the term “administered in combination” or “combined administration” means that two or more agents are administered to a subject at the same time or within an interval such that there may be an overlap of an effect of each agent on the subject in need thereof. In some embodiments, the agents are administered within about 1 or more weeks, 1 or more days, 1 or more hours, or 1 or more minutes of one another. In some embodiments, the administrations of the agents are spaced sufficiently closely together such that a combinatorial (e.g., a synergistic) effect is achieved.

As used herein, the term “pharmaceutically acceptable” refers to those compounds, materials, compositions, and/or dosage forms which are, within the scope of sound medical judgment, suitable for use in contact with the tissues a subject, such as a mammal (e.g., a human) without excessive toxicity, irritation, allergic response, or other problem or complication, commensurate with a reasonable benefit/risk ratio.

As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

### DETAILED DESCRIPTION

Described herein are compositions and methods for use in codon optimization technology. For example, the compositions and methods described herein are directed to improving mRNA stability and half-life for improved production of exogenous proteins.

Advantageously, the methods described herein enable improved production of recombinant or exogenous proteins by modifying polynucleotide sequences that encode the protein of interest such that m6A methylation of the transcribed mRNA sequence is reduced, thereby enhancing mRNA stability and yielding more robust protein expression compared to traditional methods of protein production. For example, the methods described herein may be applied to express proteins in vitro, in vivo, and/or ex vivo for therapeutic purposes, such as gene therapy, drug delivery, and/or vaccine development.

### I. Methods of Codon Optimization

The methods of codon optimization described herein may be used alone or in combination for improved protein expression in applications directed to transgene expression or heterologous gene expression. In general, codon optimization improves the translation efficiency of a target gene or transcript into a polypeptide or protein product for higher protein yield. The methods of codon optimization described herein may be applied to a gene or polynucleotide sequence that encodes any type of protein, including a soluble protein, a transmembrane protein, a membrane-associated protein, an intracellular protein, or a secreted protein, among others. In some embodiments, the methods of codon optimization described herein are applied to a gene or polynucleotide sequence that encodes a whole protein (e.g., the complete amino acid sequence), a polypeptide, or a protein fragment (e.g., one or more protein domains, a polypeptide of a protein, and/or one or more protein chains). In further embodiments, the methods of codon optimization described herein are applied to a gene or polynucleotide sequence that encodes a fusion protein (e.g., an Fc-fusion protein or an albumin fusion protein), which may further enhance protein stability and/or protein half-life.

### A. Reducing or Eliminating DRACH Motifs

In the methods described herein, increased protein expression of a protein of interest may be achieved by codon optimization techniques that reduce or eliminate DRACH motifs in a gene or coding sequence of a target of interest. A DRACH motif refers to a short, five base pair nucleic acid consensus sequence, wherein “D” designates guanine, adenine, or uracil; “R” designates guanine or adenine; “A” designates adenine; “C” designates cytosine; and “H” designates adenine, uracil, or cytosine. DRACH motifs include the following unique sequences: AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC.

A DRACH motif has a higher likelihood of undergoing the N(6)-methyladenosine (“m6A”) modification on the adenosine base in the third position, which in turn reduces the half-life of a target mRNA in a cell or a biological sample that contains mRNA. By modifying a gene or coding sequence to reduce or eliminate one or more DRACH motifs, the resulting mRNA transcript may have an increased half-life due to fewer m6A modifications as compared to the unmodified gene or coding sequence. In some embodiments, an increased half-life of the modified mRNA transcripts may be due to increased stability or reduced decay. For example, proteins of the YTHDF protein family (e.g., YTHDF2 and YTHDC2) recognize m6A-modified mRNA transcripts in the cytosol and mediate decay of these modified transcripts by destabilizing the m6A-modified mRNA transcripts and/or recruiting exonucleases.

A DRACH motif has a higher propensity or likelihood to receive an m6A modification, as demonstrated by methylation enrichment studies. The propensity of a DRACH motif undergoing an m6A modification may depend on the primary sequence of the motif, the location of the motif in the gene or coding sequence, gene or sequence topology such as secondary or tertiary structural elements surrounding the motif, or a combination thereof. For example, one or more of these factors may increase the accessibility of the DRACH sequence to a methyltransferase such as METTL3, METTL14, WTAP, KIAA1439, METTL16, RBM15, and/or ZC3H13 for m6A modification. A DRACH motif with the nucleotide sequence selected from the group AGACA, AGACT, GAACC, GAACT, GGACA, GGACT, and TGACT. has a greater likelihood of undergoing an m6A modification. Sequence-dependent enrichment of m6A modifications in DRACH motifs has been covered elsewhere such as in Schwartz et al. (Cell Rep. 8 (1): 284-296, 2014), hereby incorporated by reference. Polynucleotide topology and sequence elements also influence the enrichment of m6A modified DRACH motifs in an mRNA transcript. For example, m6A modifications may be enriched in DRACH motifs in coding sequence of a gene, the 3′ untranslated region, and/or close to stop codons. In contrast, a DRACH motif has a lower propensity of undergoing an m6A modification if the DRACH motif is in closer proximity to a splice junction or in the 5′ untranslated region. In some embodiments, a DRACH motif is less likely to receive an m6A modification if it is within about 50 nucleotides or less (e.g., about 60, about 55, about 50, about 45, about 40, about 35, about 30, about 25, about 20, about 15, about 10, or about 5 nucleotides) of a splice junction.

The presence of an m6A modification in a DRACH motif of an RNA molecule may be detected based on methods known in the art such as RNA crosslinking and immunoprecipitation techniques coupled with next-generation sequencing modalities as well as nanopore sequencing. Methods of detecting m6A modifications in mRNA transcripts are known in the art and have been described in detail in, e.g., Meyer et al. (Cell. 149 (7): 1635-1646, 2012), Dominissini et al. (Nature. 29 (485): 201-206, 2012), Linder et al. (Nat Methods. 12 (8): 767-772, 2015), Ke et al. (Genes Dev. 31 (10): 990-1006, 2017), and Lorenz et al. (RNA. 26:19-28, 2020), each of which are hereby incorporated by reference.

In some embodiments, a gene or coding sequence is modified so that between 1 and 5 DRACH motifs (e.g., 1, 2, 3, 4, or 5 DRACH motifs) are removed from a gene or coding sequence. In some embodiments, a gene or coding sequence is modified so that between 5 and 10 DRACH motifs (e.g., 5, 6, 7, 8, 9, or 10 DRACH motifs) are removed from a gene or coding sequence. In some embodiments a gene or coding sequence is modified so that between 10 and 20 DRACH motifs (e.g., 10, 11, 12, 13, 14, 15, 16, 17, 18, 19, or 20 DRACH motifs) are removed from a gene or coding sequence. In some embodiments, a gene or coding sequence is modified so that between 20 and 40 DRACH motifs (e.g., 20 to 25, 25 to 30, 30 to 35, 30 to 40; e.g., 21, 22, 23, 24, 25, 26, 27, 28, 29, 30, 31, 32, 33, 34, 35, 36, 37, 38, 39, or 40 DRACH motifs) are removed from a gene or coding sequence. In some embodiments, a gene or coding sequence is modified so that between 40 and 80 DRACH motifs (e.g., 40 to 45, 45 to 50, 50 to 55, 55 to 60, 60 to 65, 65 to 70, 70 to 75, 75 to 80; e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 DRACH motifs) are removed from a gene or coding sequence. In some embodiments, a gene or coding sequence is modified so that between 50 and 100 DRACH motifs (e.g., 50 to 60, 60 to 70, 70 to 80, 80 to 90, 90 to 100; e.g., 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DRACH motifs) are removed from a gene or coding sequence. In other embodiments, more than 100 DRACH motifs (e.g., more than 100, more than 110, more than 120, more than 130, more than 140, more than 150, more than 160, more than 170, more than 180, more than 190, or more than 200 DRACH motifs) are removed from a gene or coding sequence.

A gene or coding sequence may be modified such that all DRACH motifs are removed or eliminated from a gene or coding sequence. In some embodiments, a gene or coding sequence is modified such that the number of DRACH motifs in a sequence is reduced by at least 90%, at least 80%, at least 70%, at least 60%, at least 50%, at least 40%, at least 30%, at least 20%, or at least 10% as compared to the native gene or coding sequence.

Methods to remove one or more DRACH motifs may be combined with additional codon usage practices to further reduce the likelihood of m6A modification of a sequence. In some embodiments, the additional codon usage practice for reducing m6A modification employs one or more of the following six guidelines: (1) never use GAC for aspartic acid (Asp or D); (2) never use GAA for glutamic acid (Glu or E); (3) never use GGA for glycine (Gly or G); (4) never use AAA for lysine (Lys or K); (5) never use AAC for asparagine (Asn or N); and/or (6) always use ACG for threonine (Thr or T).

In some embodiments, removal of one or more DRACH motifs requires iteratively modifying a polynucleotide of interest to avoid introducing new DRACH motifs and/or to preserve amino acid sequence identity. In some embodiments, removal of one or more DRACH motifs involves replacing one or more codons in the polynucleotide sequence with a synonymous codon (i.e., a codon that encodes the same amino acid sequence). In some embodiments, removal of one or more DRACH motifs involves substituting nucleotides such that the primary sequence (i.e., amino acid sequence) of a polypeptide or protein has 100% sequence identity.

Other considerations that may influence mRNA stability or total protein production include mRNA GC content, mRNA secondary structure, accessibility of ribosomal binding sites, relative tRNA abundance, among other considerations known in the art. In some embodiments, higher GC content in a coding sequence improves mRNA stability and half-life by inhibiting mRNA decay and repression mechanisms in the cell (e.g., localization to P-body granules or reduced interactions with translational repressors). Optimal GC content for improved protein production may be between 50% and 80% GC content (e.g., between 50-60%, between 60-70%, or between 70-80%; e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80%) over the length of the coding sequence of interest. Structural considerations for an mRNA transcript include secondary structures such as hairpin or stem loops that may inhibit translation initiation or promote ribosome stalling. Hairpin or stem loops may form when intramolecular hydrogen bonds form between complementary nucleotides (e.g., A and T, A and U, or G and C). Mechanisms of ribosome scanning and ribosome fidelity that influence protein production are known in the art and are covered elsewhere, such as e.g., Zaher and Green (Cell. 136 (4): 746-762, 2009), which is hereby incorporated by reference. The method of codon optimization wherein one or more DRACH motifs are removed may be iteratively performed to achieve an optimal value of any one or combination of the foregoing considerations. In some embodiments, a method of codon optimization is performed 1 time, 2 times, 3 times, or more than 3 times to achieve optimal mRNA GC content and structural considerations.

In some embodiments, codon optimization by removal or elimination of one, more than one, or all present DRACH motifs leads to enhanced mRNA half-life (e.g., enhanced stability) of a gene or coding sequence of interest. In some embodiments, elimination of one, more than one, or all present DRACH motifs in a gene or coding sequence of interest increases the mRNA half-life by about 1.25-fold, by about 1.5-fold, by about 1.75-fold, by about 2-fold, by about 2.25-fold, by about 2.5-fold, by about 2.75-fold, by about 3-fold, or more as compared to a polynucleotide sequence in which the DRACH motifs has not been removed, such as the native gene or coding sequence. In some embodiments, elimination of one, more than one, or all present DRACH motifs in a gene or coding sequence of interest increases the mRNA half-life by about 5-fold to about 50-fold (e.g., by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold), as compared to a polynucleotide sequence in which the DRACH motifs has not been removed, such as the native gene or coding sequence.

In some embodiments, elimination of one, more than one, or all present DRACH motifs in a gene or coding sequence of interest increases the mRNA half-life by at least 10%, by at least 20%, by at least 30%, by at least 40%, by at least 50%, by at least 60%, by at least 70%, by at least 80%, by at least 90%, by at least 100%, or more than 100%, as compared to a polynucleotide sequence in which the DRACH motifs has not been removed, such as the native gene or coding sequence. In some embodiments, elimination of one, more than one, or all present DRACH motifs in a gene or coding sequence of interest increases the mRNA half-life, e.g., by about 1 to 24 hours, or more (e.g., about 1 hour, about 2 hours, about 3 hours, about 4 hours, about 5 hours, about 10 hours, about 15 hours, about 20 hours, about 24 hours, or more), as compared to a polynucleotide sequence in which the DRACH motifs has not been removed, such as the native gene or coding sequence. Methods of measuring mRNA half-life or stability include those known in the art and described herein.

In some embodiments, elimination of one, more than one, or all present DRACH motifs in a gene or coding sequence of interest increases the production of protein or fragment thereof (e.g., protein expression). In some embodiments, elimination of one, more than one, or all present DRACH motifs in a gene or coding sequence of interest increases the protein expression by about 1.5-fold, by about 2-fold, by about 3-fold, by about 4-fold, by about 5-fold, or more than 5-fold, as compared to a polynucleotide sequence in which the DRACH motifs has not been removed, such as the native gene or coding sequence, such as the native gene or coding sequence of interest. In some embodiments, elimination of one, more than one, or all present DRACH motifs in a gene or coding sequence of interest increases the protein expression by about 5-fold to about 50-fold (e.g., by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold), as compared as compared to a polynucleotide sequence in which the DRACH motifs has not been removed, such as the native gene or coding sequence, such as the native gene or coding sequence.

In some embodiments, elimination of one, more than one, or all present DRACH motifs in a gene or coding sequence of interest increases the protein expression by at least 10%, by at least 20%, by at least 30%, by at least 40%, by at least 50%, by at least 60%, by at least 70%, by at least 80%, by at least 90%, by at least 100%, or more than 100%, as compared to a polynucleotide sequence in which the DRACH motifs has not been removed, such as the native gene or coding sequence, such as the native gene or coding sequence. In some embodiments, an increase in the expression of a protein of interest or a fragment thereof is defined by an increase in total protein yield from recombinant production or an increase in measured protein in a biological sample. Methods of measuring protein expression are known in the art and are described herein.

### B. Codon Optimization Based on Codon Usage Bias

In some embodiments, a method of heterologous gene expression by reducing or eliminating DRACH motifs may further include codon optimization based on codon usage bias. Codon usage bias refers to the biological phenomenon in which there is preferential usage of a specific codon over other synonymous codons, which can influence rates and efficacy of protein translation. Codon usage bias may influence RNA processing, ribosomal scanning, translation initiation or termination, and/or protein folding and protein stability. Codon usage bias varies depending on the organism encoding the sequence due to evolutionary impacts on an organism's genome, including mutations, rates of recombination, and genetic drift. Varying codon usage biases are observed in organisms of different species, families, or groups and even in different genes in the same species. Therefore, codon usage bias may be considered for applications of heterologous gene expression. For example, a polynucleotide sequence that is native to a human cell may yield less abundant protein when produced in an Escherichia coli cell as compared to the human cell if it is not first modified based on codon usage in E. coli.

In some embodiments, codon optimization for heterologous gene expression involves the use of a codon usage frequency table for a given organism. An example of a codon usage frequency table for a human is shown in Table 1 below.

TABLE 1
Exemplary Codon Usage Frequency Table

| Codon | Amino Acid | Frequency |
|---|---|---|
| TGA | * | 0.52 |
| TAA | * | 0.28 |
| TAG | * | 0.20 |
| GCC | A (Ala) | 0.40 |
| GCT | A (Ala) | 0.26 |
| GCA | A (Ala) | 0.23 |
| GCG | A (Ala) | 0.11 |
| TGC | C (Cys) | 0.55 |
| TGT | C (Cys) | 0.45 |
| GAC | D (Asp) | 0.54 |
| GAT | D (Asp) | 0.46 |
| GAG | E (Glu) | 0.58 |
| GAA | E (Glu) | 0.42 |
| TTC | F (Phe) | 0.55 |
| TTT | F (Phe) | 0.45 |
| GGC | G (Gly) | 0.34 |
| GGA | G (Gly) | 0.25 |
| GGG | G (Gly) | 0.25 |
| GGT | G (Gly) | 0.16 |
| CAC | H (His) | 0.59 |
| CAT | H (His) | 0.41 |
| ATC | I (Ile) | 0.48 |
| ATT | I (Ile) | 0.36 |
| ATA | I (Ile) | 0.16 |
| AAG | K (Lys) | 0.58 |
| AAA | K (Lys) | 0.42 |
| CTG | L (Leu) | 0.41 |
| CTC | L (Leu) | 0.20 |
| TTG | L (Leu) | 0.13 |
| CTT | L (Leu) | 0.13 |
| TTA | L (Leu) | 0.07 |
| CTA | L (Leu) | 0.07 |
| ATG | M (Met) | 1.0 |
| AAC | N (Asn) | 0.54 |
| AAT | N (Asn) | 0.46 |
| CCC | P (Pro) | 0.33 |
| CCT | P (Pro) | 0.28 |
| CCA | P (Pro) | 0.27 |
| CCG | P (Pro) | 0.11 |
| CAG | Q (Gln) | 0.75 |
| CAA | Q (Gln) | 0.25 |
| CGG | R (Arg) | 0.21 |
| AGA | R (Arg) | 0.20 |
| AGG | R (Arg) | 0.20 |
| CGC | R (Arg) | 0.19 |
| CGA | R (Arg) | 0.11 |
| CGT | R (Arg) | 0.08 |
| AGC | S (Ser) | 0.24 |
| TCC | S (Ser) | 0.22 |
| TCT | S (Ser) | 0.18 |
| AGT | S (Ser) | 0.15 |
| TCA | S (Ser) | 0.15 |
| TCG | S (Ser) | 0.06 |
| ACC | T (Thr) | 0.36 |
| ACA | T (Thr) | 0.28 |
| ACT | T (Thr) | 0.24 |
| ACG | T (Thr) | 0.12 |
| GTG | V (Val) | 0.47 |
| GTC | V (Val) | 0.24 |
| GTT | V (Val) | 0.18 |
| GTA | V (Val) | 0.11 |
| TGG | W (Tryp) | 1.0 |
| TAC | Y (Tyr) | 0.57 |
| TAT | Y (Tyr) | 0.43 |

Codon: A = adenine, C = cytosine, G = guanine, T = thymine; Amino Acid: * = stop codon, A = alanine, C = cysteine, D = aspartic acid, E = glutamic acid; F = phenylalanine; G = glycine, H = histidine, I = isoleucine, K = lysine, L = leucine, M = methionine, N = asparagine, P = proline, Q = glutamine, R = arginine, S = 5 serine, T = threonine, V = valine, W = tryptophan, Y = tyrosine; Reported frequency corresponds to human codons

A method of codon optimization for increasing protein expression of a target of interest may be achieved by modifying the gene or coding sequence to replace one or more codons with the synonymous codon having the highest usage frequency. For example, a gene or coding sequence for protein expression in a human may be modified such that only the codons with the highest frequency as shown in Table 1 are represented at every instance for a particular amino acid in the modified sequence (e.g., all codons encoding leucine are modified to CTG).

In some embodiments, a gene or coding sequence for protein expression may be modified such that all recited codons are replaced with synonymous codons that have the highest usage frequency in an organism. In some embodiments, a gene or coding sequence for protein expression may be modified such that between 5% and 50% of recited codons (e.g., between 5% and 10%, between 10% and 25%, between 20% and 40%, or between 25% and 50%; e.g., about 5%, about 10%, about 15%, about 20%, about 25%, about 30%, about 35%, about 40%, about 45%, or about 50%) are replaced with synonymous codons that have the highest usage frequency. In some embodiments, a gene or coding sequence for protein expression may be modified such that between 25% and 75% (e.g., between 25% and 40%, between 30% and 50%, between 40% and 60%, or between 50% and 75%; e.g., about 25%, about 30%, about 35%, about 40%, about 45%, about 50%, about 55%, about 60%, about 65%, about 70%, or about 75%) of recited codons are replaced with synonymous codons that have the highest usage frequency. In some embodiments, a gene or coding sequence for protein expression may be modified such that between 75% and 100% (e.g., between 75% and 85%, between 80% and 90%, between 85% and 95%, between 90% and 100%; e.g., about 75%, about 80%, about 85%, about 90%, about 95%, or about 100%) of recited codons are replaced with synonymous codons that have the highest usage frequency.

A method of codon optimization for increasing protein expression of a target of interest may be achieved by modifying the gene or codon sequence such that one or more codons are represented proportional to their reported usage frequency in an organism. For instance, a gene or codon sequence for protein expression in a human may be modified such that codons encoding a particular amino acid are represented according to their usage frequency as shown in Table 1 (e.g., codons encoding leucine are modified such that CTG is represented in about 41% of instances, CTC is represented in about 20% of instances, TTG is represented in about 13% of instances, etc.).

In some embodiments, a gene or coding sequence for protein expression may be modified such that all recited codons are replaced with synonymous codons based on their proportional usage frequency. In some embodiments, a gene or coding sequence for protein expression may be modified such that between 5% and 50% of recited codons (e.g., between 5% and 10%, between 10% and 25%, between 20% and 40%, or between 25% and 50%; e.g., about 5%, about 10%, about 15%, about 20%, about 25%, about 30%, about 35%, about 40%, about 45%, or about 50%) are replaced with synonymous codons based on their proportional usage frequency. In some embodiments, a gene or coding sequence for protein expression may be modified such that between 25% and 75% (e.g., between 25% and 40%, between 30% and 50%, between 40% and 60%, or between 50% and 75%; e.g., about 25%, about 30%, about 35%, about 40%, about 45%, about 50%, about 55%, about 60%, about 65%, about 70%, or about 75%) of recited codons are replaced with synonymous codons based on their proportional usage frequency. In some embodiments, a gene or coding sequence for protein expression may be modified such that between 75% and 100% (e.g., between 75% and 85%, between 80% and 90%, between 85% and 95%, between 90% and 100%; e.g., about 75%, about 80%, about 85%, about 90%, about 95%, or about 100%) of recited codons are replaced with synonymous codons based on their proportional usage frequency.

In some embodiments, the method of codon optimization is performed using available software or webtools known in the art, wherein the codon optimization is automated or performed based on an algorithm. In some embodiments, the method of codon optimization involves a heuristic scoring method. In some embodiments, the method of codon optimization involves the use of a neural network. In some embodiments, the method of codon optimization is performed with quantum computing (see, e.g., Fox et al. PLOS ONE 16 (10): e0259101, 2021, hereby incorporated by reference). In some embodiments, a method of codon optimization is iteratively performed to achieve optimal mRNA guanine-cytosine (GC) content, mRNA secondary structure, mRNA motifs, ribosomal binding sites among other considerations.

### C. Evaluating Methods of Codon Optimization

A method of codon optimization may be evaluated by measuring mRNA stability or half-life of the modified gene or coding sequence, as compared to the native gene or coding sequence. In some embodiments, one or more methods of codon optimization described herein, or a combination thereof yields to an mRNA transcript with increased stability or an increased half-life. In some embodiments, mRNA half-life is evaluated by one or more measurements of the concentration, relative abundance, or relative stability of an mRNA transcript detected in a biological sample. The concentration, relative abundance, or relative stability of an mRNA transcript may be evaluated by reverse transcription-polymerase chain reaction (RT-PCR), fluorimetry, colorimetry, spectrophotometric analyses, flow cytometry, in situ hybridization methods, Northern blot analyses, or an array using one or more hybridizing oligonucleotides or antibodies against one or more mRNA transcripts of interest. In further embodiments, the half-life or stability of an mRNA transcript of interest is evaluated by structural or biochemical methods. In some embodiments, the half-life or stability of an mRNA transcript is evaluated by selective 2′-hydroxyl acylation analyzed by primer extension (SHAPE) or modified methodologies thereof (see, e.g., Wilkinson et al. (Nat. Prot. 1:1610-1616, 2006, hereby incorporated by reference). In further embodiments, the half-life or stability of an mRNA transcript of interest may be evaluated by mass spectrometry to assess the presence of chemical modifications. One or more of the methods of evaluating mRNA half-life or stability as described herein may be used in any combination, optionally in addition to other methods known in the art.

In some embodiments, the half-life or stability of an mRNA transcript of interest is evaluated by pulse-chase methodologies to analyze mRNA decay. In some embodiments, the mRNA half-life of an mRNA transcript of interest is evaluated by measuring the rate of mRNA decay after the inhibition of transcription (e.g., by application of actinomycin D to a biological sample in which the biological sample is derived from a subject or a population of cells cultured in vitro). In any one of the foregoing embodiments, the stability or half-life of an mRNA transcript from a modified gene or coding sequence may be compared to a reference sample. In some embodiments, the reference sample derives from the native gene or coding sequence. In other embodiments, the reference sample is a house-keeping gene (e.g., a gene associated with viability that is stably expressed in a cell; e.g., beta-actin, GAPDH, rRNA).

Additionally or alternatively, the method of codon optimization may be evaluated by measuring protein production by the modified gene or coding sequence, as compared to the native gene or coding sequence. In some embodiments, protein production is evaluated by measuring the concentration of a protein of interest in a sample. In some embodiments, the concentration of a protein of interest is measured by mass spectrometry, Western blot analysis, ELISA, immunoprecipitation methods, flow cytometry, fluorimetry, colorimetry, spectrophotometric analyses, or an array using binding oligonucleotides such as aptamers or antibodies against one or more proteins of interest.

The method of codon optimization may be evaluated based on protein quality or protein stability of the produced protein or polypeptide of interest. Protein quality or stability may be measured by functional or binding assays specific to the protein of interest (e.g., an enzyme activity assay or a binding assay using one or more known binding partners as a ligand or analyte). Protein quality or stability may also be evaluated using biochemical or biophysical methods known in the art such as, e.g., differential scanning fluorimetry, thermal shift assays, circular dichroism, dynamic light scattering, analytical ultracentrifugation, size exclusion chromatography, isothermal titration calorimetry, microscale thermophoresis, mass spectrometry, hydrogen-deuterium exchange, among other methods known in the art (see, e.g., Le Basle et al. (J. Pharm. Sci. 109 (1): 169-190, 2020), hereby incorporated by reference).

### II. Applications of Methods of Codon Optimization

### A. Methods of Protein Production

Any of the codon optimization methods described herein may be used alone or in combination for heterologous or transgenic gene expression in a host cell such as a eukaryotic cell or a prokaryotic cell. In some embodiments, the host cell is a bacterial cell, a yeast cell, an insect cell, a plant cell, or a mammalian cell. In further embodiments the host cell is cultured in vitro for heterologous gene expression or recombinant protein production. In some embodiments, the host cell is cultured as an adherent cell or as a cell in suspension. In some embodiments, the host cell is an established model cell line, such as a cell line for culturing available through the American Type Culture Collection (ATCC).

In some embodiments, the host cell is in an organism for production of a protein in vivo (e.g., transgenic expression). In some embodiments, the host cell derives from an organism (e.g., a human subject) for in vitro culture methods, wherein following in vitro culturing, the host cell may be implanted, injected, deposited, or otherwise administered to the same or a different organism (e.g., for autogenic or allogenic therapies or treatments).

### B. Methods of Delivering a Codon Optimized Gene Product

A codon optimized gene product (e.g., a transgene, an mRNA transcript, or a purified or partially purified protein) may be delivered to a host cell or a subject by way of a variety of delivery techniques. The sections that follow describe exemplary, non-limiting modalities for delivering a polynucleotide of interest (e.g., a polynucleotide in which one or more DRACH motifs have been eliminated in accordance with the methods described herein) to a host cell or subject.

i. Viral Genomes for Delivering a Codon Optimized Gene Product

Viral genomes provide a rich source of vectors that can be used for the efficient delivery of a gene of interest into the genome of a target cell in a host cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors that may be used in conjunction with the compositions and methods described herein are adeno-associated virus (AAV), retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses that may be used in conjunction with the compositions and methods described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

ii. AAV Vectors for Delivering a Codon Optimized Gene Product

In some embodiments, a codon optimized polynucleotide (e.g., a modified gene or coding sequence) described herein is incorporated into recombinant AAV (rAAV) vector in order to facilitate introduction into a cell. rAAV vectors useful in the conjunction with the compositions and methods described herein include recombinant nucleic acid constructs that contain (1) a transgene encoding a codon optimized sequence for enhanced protein production and (2) one or more nucleic acids that facilitate expression of the modified gene or coding sequence. The viral nucleic acids may include those cis-acting elements of rAAV for replication and packaging (e.g., functional inverted terminal repeats, or “ITRs”) of the DNA into a virion. Such rAAV vectors may also contain marker or reporter genes. Useful rAAV vectors include those having one or more of the naturally-occurring AAV genes deleted in whole or in part, but retain functional flanking ITR sequences. The AAV ITRs may be of any serotype (e.g., derived from serotype 2) suitable for a particular application. Methods for using rAAV vectors are described, for example, in Tal et al. (J. Biomed. Sci. 7:279-291, 2000), and Monahan and Samulski (Gene Delivery. 7:24-30, 2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al. (J. Virol. 76:791-801, 2002) and Bowles et al. (J. Virol. 77:423-432, 2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, and 9, among others. Construction and use of AAV vectors and AAV proteins of different serotypes are described, for example, in Chao et al., Mol. Ther. 2:619-623, 2000), Davidson et al. (Proc. Natl. Acad. Sci. USA 97:3428-3432, 2000), Xiao et al. (J. Virol. 72:2224-2232, 1998), Halbert et al. (J. Virol. 74:1524-1532, 2000); Halbert et al. (J. Virol. 75:6615-6624, 2001), and Auricchio et al. (Hum. Molec. Genet. 10:3075-3081, 2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV2) pseudotyped with a capsid protein derived from a serotype other than the given serotype (e.g., AAV1, AAV3, AAV4, AAV5, AAV6, AAV7, AAV8, or AAV9, among others). For example, a representative pseudotyped vector is an AAV2 vector encoding a therapeutic protein pseudotyped with a capsid protein derived from AAV serotype 8 or AAV serotype 9. In some embodiments, the pseudotyped AAV has the ITRs of one AAV serotype (e.g., AAV2) and the VP1, VP2, and/or VP3 capsid proteins from a different AAV serotype (e.g., AAV1, AAV2, AAV3, AAV4, AAV5, AAV6, AAV7, AAV8, AAV9, AAVrh10, or AAVrh74). Techniques involving the construction and use of pseudotyped rAAV virions are known in the art and are described, for example, in Duan et al., J. Virol. 75:7662-7671 (2001); Halbert et al. (J. Virol. 74:1524-1532, 2000); Zolotukhin et al. (Methods. 28:158-167, 2002); and Auricchio et al. (Hum. Molec. Genet. 10:3075-3081, 2001).

In some embodiments, the AAV comprises a capsid disclosed, e.g., in WO 2017/218842, the disclosure of which is incorporated herein by reference. In some embodiments, the AAV comprises a capsid protein disclosed in Lin et al. (Mol Brain. 13:138, 2020), the disclosure of which is incorporated herein by reference. In some embodiments, the AAV comprises an AAV2-retro or an AAV9-retro capsid protein. In some embodiments, the AAV comprises a capsid protein that is conjugated to a ligand or an aptamer.

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al. (J. Virol. 74:8635-45, 2000). Other rAAV virions that can be used in methods of the invention include those capsid hybrids that are generated by molecular breeding of viruses as well as by exon shuffling. See, e.g., Soong et al. (Nat. Genet. 25:436-439, 2000) and Kolman and Stemmer (Nat. Biotechnol. 19:423-428, 2001).

iii. Additional Methods of Delivering a Codon Optimized Gene Product to a Host Cell

In addition to the virus-based delivery modalities described above, a variety of non-viral techniques can also be used to introduce a codon optimized gene or coding sequence into a subject or host cell (e.g., a host cell derived from a human subject). For example, electroporation can be used to permeabilize mammalian cells (e.g., human cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al. (Nucleic Acids Res. 15:1311, 1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al. (Exp. Dermatol. 14:315, 2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al. (J. Vis. Exp. 81: e50980, 2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is herein incorporated by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig (Topics in Current Chemistry 228:227, 2003), the disclosure of which is incorporated herein by reference) and diethylaminoethyl (DEAE)-dextran, the use of which as a transfection agent is described in detail, for example, in Gulick et al. (Curr. Protoc. in Mol. Biol. 40:1:9.2:9.2.1, 1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al. (Methods in Cell Biology 82:309, 2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Mol. Ther. 23: Supplement 1, Abstract No. 122, 2015).

### C. Methods of Treatment

i. Therapeutic Utility of Codon-Optimized Gene Sequences

The methods of codon optimization and protein production described herein may be applied to methods of treating a disease or condition in a subject in need thereof. In some embodiments, a method of treatment may be prophylactic treatment for a subject at risk of a disease or condition. In other embodiments, a method of treatment may reduce, reverse, ameliorate, stabilize, or improve a disease state or condition in a subject. In other embodiments, the methods may be used to alleviate, ameliorate, reduce, or reverse clinical manifestations of a disease or condition.

In some embodiments, the method of treatment is intended to treat a disease or condition caused by a defect or deficiency in a single gene or single gene product (e.g., a single polynucleotide or protein). In some embodiments, the method of treatment is intended to treat a disease or condition that leads to a defect or deficiency in multiple gene products. In some embodiments, the defect or deficiency is defined by reduced expression, reduced activity, and/or aberrant localization of the one or more gene products. In some embodiments, the gene product is delivered for transient expression. In other embodiments, the gene product integrates into the host genome.

In some embodiments, the method of treatment is intended to replace, supplement, or replenish an absent, deficient (e.g., low expression levels), or defective (e.g., mutant, loss-of-function, or low biological or catalytic activity) gene product in a subject having a disorder or condition. In some embodiments, the disorder or condition is characterized by a loss-of-function mutation or a gene deletion. In other embodiments, the disorder or condition is acquired (e.g., a deficient or defective gene product from stochastic or environmental factors). In some embodiments, the codon optimized gene product encodes a polypeptide or protein that is identical (i.e., retains 100% sequence identity) to the wild-type amino acid sequence to replace, supplement, or replenish low levels of the one or more deficient or defective gene products. In other embodiments the codon optimized gene product encodes a polypeptide or protein that shares at least 85% (e.g., 85%, 86%, 87%, 88%, 89%, 90%, 91%, 92%, 93%, 94%, 95%, 96%, 97%, 98%, or 99%) sequence identity to the wild-type amino acid sequence to encode a polypeptide or protein that has improved biological function (e.g., increased catalytic function or reduced immunogenicity). Some examples in which a polypeptide or protein may be modified for improved biological function include mutating or adding sites for post-translational modifications (e.g., glycosylation, phosphorylation, ubiquitination, nitrosylation, methylation, acetylation, lipidation, among others, or a combination thereof), mutating or adding cysteines for altered or added disulfide bonds, modifying binding sites for enhanced binding activity between the protein of interest and one or more known binding partners, modifying sites of protease binding or targeted cleavage, modifying a signal sequence for enhanced secretion or altered localization, among other protein modifications known in the art.

In some embodiments, the method of treatment is intended to increase or supplement the expression of a normally expressing gene product (e.g., is present at a concentration within an accepted healthy range) in a subject having a disorder or condition that would benefit from increased expression of said gene product. Increased expression of a normally expressing gene product may be desired in order to increase the rate of an enzymatic reaction, enhance the potency or rate of a signaling response (e.g., intracellularly or extracellularly), modulate trafficking or adhesion of a cell or cellular component, increase the likelihood or propensity of binding or a transient interaction to occur (e.g., based on the affinity or K_{D} of two or more molecules), or otherwise modulate one or more biological processes. Such methods may be clinically useful for increasing the expression of a protein with a redundant function to a deficient or defective protein. Such methods may also be clinically useful for modulating a disease-causing gene product that is logistically more difficult to employ as a codon optimized gene product for a method of treatment due to an assortment of non-limiting factors including large gene size, low accessibility of a target cell or tissue, and/or high immunogenicity of the gene product.

In some embodiments, the method of treatment increases the expression of a protein or polypeptide (e.g., upregulates, induces the expression of an exogenous protein or polypeptide) to modulate or regulate a separate causative agent underlying a disease. Such embodiments may be clinically useful for blocking, inhibiting, proteolyzing, mediating clearance of, or otherwise attenuating the effect of a causative agent such as a pathogen (e.g., a virus, a bacterium, a fungus, or a parasite) or a pro-inflammatory protein (e.g., a cytokine or a cytokine receptor).

In some embodiments, treatment with an effective amount of a codon optimized gene product (e.g., a polynucleotide encoding a protein of interest; e.g., a transgene packaged into an AAV for encoding a protein of interest) or a pharmaceutical composition containing the same increases the expression of the gene product by about 5% to 50% (e.g., about 5%, about 10%, about 15%, about 20%, about 25%, about 30%, about 35%, about 40%, about 45%, or about 50%), by about 50% to 100% (about 50%, about 55%, about 60%, about 65%, about 70%, about 75%, about 80%, about 85%, about 90%, about 95%, or about 100%), or greater than 100% (about 100%, about 125%, about 150%, about 175%, about 200%, about 225%, about 250%, about 275%, about 300%, or more) as compared to a reference sample such as a biological sample from a healthy control subject or a biological sample of the same subject prior to the administration of treatment. In some embodiments, treatment with an effective amount of a codon optimized gene product (e.g., a polynucleotide encoding a protein of interest; e.g., a transgene packaged into an AAV for encoding a protein of interest) increases the expression of the gene product by about 1-fold, by about 2-fold, by about 3-fold, by about 4-fold, by about 5-fold, by about 6-fold, by about 7-fold, by about 8-fold, by about 9-fold, by about 10-fold, by about 11-fold, by about 12-fold, by about 13-fold, by about 14-fold, by about 15-fold, by about 16-fold, by about 17-fold, by about 18-fold, by about 19-fold, by about 20-fold, about 25-fold, about 30-fold, about 35-fold, about 40-fold, about 45-fold, about 50-fold, or more as compared to a reference sample such as a biological sample from a healthy control subject or a biological sample of the same subject prior to the administration of treatment. In any of the foregoing embodiments, treatment with an effective amount of a codon optimized gene product may result in increased expression globally (e.g., in all organ systems or all tissues of a subject; e.g. throughout the body) or locally (e.g., in a subset of tissues, in one or more organs, or in one or more organ system of interest).

In some embodiments, the method of treatment is directed to treating a metabolic disorder, a blood disorder, a cardiovascular disorder, a neurological disorder, an ocular or ophthalmological disorder, a reproductive disorder, an infectious disease, an autoimmune or immunological disorder, or a type of cancer. Exemplary diseases and target genes that may benefit from the methods and applications described herein are summarized in Table 2 below.

TABLE 2
Exemplary Diseases and Target Genes

| Disease(s) | Gene(s) |
|---|---|
| Achondroplasia | FGFR3 |
| Achromatopsia | CNGA3, CNGB3, GNAT2, PDE6C, PDE6H, |
| | ACHM2, ACHM3 |
| Acute Renal Injury | NFkappaB, AATF, p85alpha, FAS, Apoptosis |
| | cascade elements (e.g., FASR, Caspase 2, 3, |
| | 4, 6, 7, 8, 9, 10, AKT, TNF alpha, IGF1, |
| | IGF1R, RIPK1), p53 |
| Age Related Macular | Abcr, CCL2, CC2, CP, Timp3, cathepsinD, |
| Degeneration | VLDLR, CCR2, sFLTOI (VEGF/PIGF (placental |
| | growth factor) binding domain of human |
| | VEGFRI/FIt-1 (hVEGFRI) fused to the Fe portion |
| | of human IgG(I) through a polyglycine linker) |
| Acquired Immune Deficiency | KIR3DL1, NKAT3, NKB1, AMB11, KIR3DS1, |
| Syndrome (AIDS) | IFNG, CXCL12, SDF1 |
| Albinism (including | TYR, OCA2, TYRP1, SLC45A2, SLC24A5, |
| oculocutaneous albinism | C10orf11 |
| (Types 1-7) and ocular | |
| albinism) | |
| Alkaptonuria | HGD |
| Alpha-1 Antitrypsin | AAT, SERPINA1, those set forth in |
| Deficiency (AATD or AIAD) | WO2017165862, PiZ allele |
| or Hereditary Emphysema | |
| Amyotrophic lateral | SOD1, ALS2, ALS3, ALS5, |
| sclerosis (ALS) | ALS7, STEX, FUS, TARDBP, VEGF (VEGF- |
| | a; VEGF-b; VEGF-c), DPP6, NEFH, PTGS1, |
| | SLC1A2, TNFRSF10B, PRPH, HSP90AA1, |
| | CRIA2, IFNG, AMPA2 S100B, FGF2, AOX1, |
| | CS, TXN, RAPHJ1, MAP3K5, NBEAL1, |
| | GPX1, ICA1L, RAC1, MAPT, ITPR2, |
| | ALS2CR4, GLS, ALS2CR8, CNTFR, |
| | ALS2CR11, FOLH1, FAM117B, P4HB, |
| | CNTF, SQSTM1, STRADB, NAIP, NLR, |
| | YWHAQ, SLC33A1, TRAK2, SCA1, NIF3L1, |
| | NIF3, PARD3B, COX8A, CDK15, HECW1, |
| | HECT, C2, WW 15, NOS1, MET, SOD2, |
| | HSPB1, NEFL, CTSB, ANG, HSPA8, RNase |
| | A, VAPB, VAMP, SNCA, alpha HGF, CAT, |
| | ACTB, NEFM, TH, BCL2, FAS, CASP3, CLU, |
| | SMN1, G6PD, BAX, HSF1, RNF19A, JUN, |
| | ALS2CR12, HSPA5, MAPK14, APEX1, |
| | TXNRD1, NOS2, TIMP1, CASP9, XIAP, |
| | GLG1, EPO, VEGFA, ELN, GDNF, NFE2L2, |
| | SLC6A3, HSPA4, APOE, PSMB8, DCTN2, |
| | TIMP3, KIFAP3, SLC1A1, SMN2, CCNC, |
| | STUB1, ALS2, PRDX6, SYP, CABIN1, |
| | CASP1, GART, CDK5, ATXN3, RTN4, C1QB, |
| | VEGFC, HTT, PARK7, XDH, GFAP, MAP2, |
| | CYCS, FCGR3B, CCS, UBL5, MMP9m |
| | SLC18A3, TRPM7, HSPB2, AKT1, DEERL1, |
| | CCL2, NGRN, GSR, TPPP3, APAF1, |
| | BTBD10, GLUD1, CXCR4, SLC1A3, FLT1, |
| | PON1, AR, LIF, ERBB3, GAS1, CD44, TP53, |
| | TLR3, GRIA1, GAPDH, AMPA, GRIK1, DES, |
| | CHAT, FLT4, CHMP2B, BAG1, CHRNA4, GSS, |
| | BAK1, KDR, GSTP1, OGG1, IL6 |
| Alzheimer's Disease | E1, CHIP, UCH, UBB, Tau, LRP, PICALM, CLU, |
| | PS1, SORL1, CR1, VLDLR, UBA1, UBA3, |
| | CHIP28, AQP1, UCHL1, UCHL3, APP, AAA, |
| | CVAP, AD1, APOE, AD2, DCP1, ACE1, |
| | MPO, PACIP1, PAXIP1L, PTIP, A2M, BDNF, |
| | BLMH, BMH, PSEN1, AD3, ALAS2, ABCA1, |
| | BIN1, BDNF, BTNL8, C1ORF49, CDH4, |
| | CHRNB2, CKLFSF2, CLEC4E, CR1L, CSF3R, |
| | CST3, CYP2C, DAPK1, ESR1, FCAR, |
| | FCGR3B, FFA2, FGA, GAB2, GALP, GAPDHS, |
| | GDNF, GMPB, HP, HTR7, IDE, IF127, IFI6, |
| | IFIT2, IL1RN, IL-1RA, IL8RA, IL8RB, JAG1, |
| | KCNJ15, LRP6, MAPT, MARK4, MPHOSPH1, |
| | MTHFR, NBN, NCSTN, NIACR2, NGF, |
| | NMNAT3, NTM, ORM1, P2RY13, PBEF1, |
| | PCK1, PICALM, PLAU, PLXNC1, PRNP, |
| | PSEN1, PSEN2, PTPRA, RALGPS2, RGSL2, |
| | SELENBP1, SLC25A37, SORL1, Mitoferrin-1, |
| | TF, TFAM, TNF, TNFRSF10C, UBE1C |
| Amyloidosis | APOA1, APP, AAA, CVAP, AD1, GSN, FGA, |
| | LYZ, TTR, PALB |
| Amyloid Neuropathy | TTR, PALB |
| Anemia | CDAN1, CDA1, RPS19, DBA, PKLR, PK1, |
| | NT5C3, UMPH1, PSN1, RHAG, RH50A, |
| | NRAMP2, SPTB, ALAS2, ANH1, ASB, ABCB7, |
| | ABC7, ASAT |
| Angelman Syndrome | UBE3A |
| Anxiety | BDNF |
| Arthritis, Rheumatoid Arthritis | TNFR:Fc |
| Attention Deficit Hyperactivity | PTCHD1 |
| Disorder (ADHD) | |
| Autoimmune Lymphoproliferative | TNFRSF6, APT1, FAS, CD95, ALPS1A |
| Syndrome | |
| Autism, Autism spectrum | PTCHD1, Mecp2, BZRAP1, MDGA2, Sema5A, |
| disorders (ASDs), including | Neurexin 1, GLO1, RTT, PPMX, MRX16, RX79, |
| Asperger's and a general | NLGN3, NLGN4, KIAA1260, AUTSX2, FMR1, |
| diagnostic category called | FMR2, FXR1, FXR2, MGLUR5, ATP10C, |
| Pervasive Developmental | CDH10, GRM6, MGLUR6, CDH9, CNTN4, |
| Disorders (PDDs) | NLGN2, CNTNAP2, SEMA5A, DHCR7, |
| | NLGN4X, NLGN4Y, DPP6, NLGN5, EN2, |
| | NRCAM, MDGA2, NRXN1, FMR2, AFF2, |
| | FOXP2, OR4M2, OXTR, FXR1, FXR2, PAH, |
| | GABRA1, PTEN, GABRA5, PTPRZ1, GABRB3, |
| | GABRG1, HIRIP3, SEZ6L2, HOXA1, SHANK3, |
| | IL6, SHBZRAP1, LAMB1, SLC6A4, SERT, |
| | MAPK3, TAS2R1, MAZ, TSC1, MDGA2, TSC2, |
| | MECP2, UBE3A, WNT2, see also 20110023145 |
| Autosomal Dominant Polycystic | PKD1, PKD2 |
| Kidney Disease (ADPKD) - | |
| (includes diseases such as | |
| von Rippel-Lindau disease | |
| and Tuberous Sclerosis Complex | |
| disease) | |
| Autosomal Recessive Polycystic | PKDH1 |
| Kidney Disease (ARPKD) | |
| Ataxia-Telangiectasia (aka | ATM |
| Louis Bar syndrome) | |
| B-Cell Non-Hodgkin Lymphoma | BCL7A, BCL7 |
| Bardet-Biedl Syndrome | ARL6, BBS1, BBS2, BBS4, BBS5, BBS7, BBS9, |
| | BBS10, BBS12, CEP290, INPP5E, LZTFL1, |
| | MKKS, MKS1, SDCCAGS, TRIM32, TTC8 |
| Bare Lymphocyte Syndrome | TAPBP, TPSN, TAP2, ABCB3, PSF2, RING11, |
| | MHC2TA, C2TA, RFX5, RFXAP, RFX5 |
| Bartter's Syndrome (Types | SLC12A1 (type I), KCNJ1 (type II), CLCNKB |
| I, II, III, IVA and B, and V) | (type III), BSND (type IV A), or both the |
| | CLCNKA CLCNKB genes (type IV B), CASR |
| | (type V) |
| Batten's Disease, Late | CLN2 |
| Infantile Neuronal Ceroid | |
| Lipofuscinosis | |
| Becker Muscular Dystrophy | DMD, BMD, MYF6 |
| Best Disease (Vitelliform | VMD2 |
| Macular Dystrophy Type 2) | |
| Bipolar Disorder | BDNF |
| Bleeding Disorders | TBXA2R, P2RX1, P2XI |
| Blue Cone Monochromacy | OPN1LW, OPN1MW, LCR |
| Breast Cancer | BRCA1, BRCA2, COX-2 |
| Bruton's Disease (aka | BTK |
| X-linked Agammglobulinemia) | |
| Canavan's disease | ASPA |
| Cancers (e.g., Lymphoma, | FAS, BID, CTLA4, PDCD1, CBLB, PTPN6, |
| Chronic Lymphocytic Leukemia | TRAC, TRBC, those described in |
| (CLL), B Cell Acute Lymphocytic | WO2015048577 |
| Leukemia (B-ALL), Acute | |
| Lymphoblastic Leukemia, Acute | |
| Myeloid Leukemia, Non-Hodgkin's | |
| Lymphoma (NHL), Diffuse Large | |
| Cell Lymphoma (DLCL), Multiple | |
| Myeloma, Renal Cell Carcinoma | |
| (RCC), Neuroblastoma, Colorectal | |
| Cancer, Breast Cancer, Ovarian | |
| Cancer, Melanoma, Sarcoma, | |
| Lung Cancer, Esophageal Cancer, | |
| Hepatocellular Carcinoma, | |
| Pancreatic Cancer, Astrocytoma, | |
| Mesothelioma, Head and Neck | |
| Cancer, and Medulloblastoma) | |
| Cardiovascular Diseases | IL1B, XDH, TP53, PTGS, MB, IL4, ANGPT1, |
| | ABCGu8, CTSK, PTGIR, KCNJ11, INS, CRP, |
| | PDGFRB, CCNA2, PDGFB, KCNJ5, KCNN3, |
| | CAPN10, ADRA2B, ABCG5, PRDX2, CPAN5, |
| | PARP14, MEX3C, ACE, RNF, IL6, TNF, STN, |
| | SERPINE1, ALB, ADIPOQ, APOB, APOE, LEP, |
| | MTHFR, APOA1, EDN1, NPPB, NOS3, PPARG, |
| | PLAT, PTGS2, CETP, AGTR1, HMGCR, IGF1, |
| | SELE, REN, PPARA, PON1, KNG1, CCL2, LPL, |
| | VWF, F2, ICAM1, TGFB, NPPA, IL10, EPO, |
| | SOD1, VCAM1, IFNG, LPA, MPO, ESR1, |
| | MAPK, HP, F3, CST3, COG2, MMP9, |
| | SERPINC1, F8, HMOX1, APOC3, IL8, PROL1, |
| | CBS, NOS2, TLR4, SELP, ABCA1, AGT, LDLR, |
| | GPT, VEGFA, NR3C2, IL18, NOS1, NR3C1, |
| | FGB, HGF, IL1A, AKT1, LIPC, HSPD1, |
| | MAPK14, SPP1, ITGB3, CAT, UTS2, THBD, |
| | F10, CP, TNFRSF11B, EGFR, MMP2, PLG, |
| | NPY, RHOD, MAPK8, MYC, FN1, CMA1, PLAU, |
| | GNB3, ADRB2, SOD2, F5, VDR, ALOX5, HLA- |
| | DRB1, PARP1, CD40LG, PON2, AGER, IRS1, |
| | PTGS1, ECE1, F7, IRMN, EPHX2, IGFBP1, |
| | MAPK10, FAS, ABCB1, JUN, IGFBP3, CD14, |
| | PDE5A, AGTR2, CD40, LCAT, CCR5, MMP1, |
| | TIMP1, ADM, DYT10, STAT3, MMP3, ELN, |
| | USF1, CFH, HSPA4, MMP12, MME, F2R, |
| | SELL, CTSB, ANXA5, ADRB1, CYBA, FGA, |
| | GGT1, LIPG, HIF1A, CXCR4, PROC, SCARB1, |
| | CD79A, PLTP, ADD1, FGG, SAA1, KCNH2, |
| | DPP4, NPR1, VTN, KIAA0101, FOS, TLR2, |
| | PPIG, IL1R1, AR, CYP1A1, SERPINA1, MTR, |
| | RBP4, APOA4, CDKN2A, FGF2, EDNRB, |
| | ITGA2, VLA-2, CABIN1, SHBG, HMGB1, |
| | HSP90B2P, CYP3A4, GJA1, CAV1, ESR2, LTA, |
| | GDF15, BDNF, CYP2D6, NGF, SP1, TGIF1, |
| | SRC, EGF, PIK3CG, HLA-A, KCNQ1, CNR1, |
| | FBN1, CHKA, BEST1, CTNNB1, IL2, CD36, |
| | PRKAB1, TPO, ALDH7A1, CX3CR1, TH, F9, |
| | CH1, TF, HFE, IL17A, PTEN, GSTM1, DMD, |
| | GATA4, F13A1, TTR, FABP4, PON3, APOC1, |
| | INSR, TNFRSF1B, HTR2A, CSF3, CYP2C9, |
| | TXN, CYP11B2, PTH, CSF2, KDR, PLA2G2A, |
| | THBS1, GCG, RHOA, ALDH2, TCF7L2, |
| | NFE2L2, NOTCH1, UGT1A1, IFNA1, PPARD, |
| | SIRT1, GNHR1, PAPPA, ARR3, NPPC, AHSP, |
| | PTK2, IL13, MTOR, ITGB2, GSTT1, IL6ST, |
| | CPB2, CYP1A2, HNF4A, SLC64A, PLA2G6, |
| | TNFSF11, SLC8A1, F2RL1, AKR1A1, |
| | ALDH9A1, BGLAP, MTTP, MTRR, SULT1A3, |
| | RAGE, C4B, P2RY12, RNLS, CREB1, POMC, |
| | RAC1, LMNA, CD59, SCM5A, CYP1B1, MIF, |
| | MMP13, TIMP2, CYP19A1, CUP21A2, PTPN22, |
| | MYH14, MBL2, SELPLG, AOC3, CTSL1, PCNA, |
| | IGF2, ITGB1, CAST, CXCL12, IGHE, KCNE1, |
| | TFRC, COL1A1, COL1A2, IL2RB, PLA2G10, |
| | ANGPT2, PROCR, NOX4, HAMP, PTPN11, |
| | SLCA1, IL2RA, CCL5, IRF1, CFAR, CACA, |
| | EIF4E, GSTP1, JAK2, CYP3A5, HSPG2, CCL3, |
| | MYDSS, VIP, SOAT1, ADRBK1, NR4A2, |
| | MMPS, NPR2, GCH1, EPRS, PPARGC1A, F12, |
| | PECAM1, CCL4, CERPINA34, CASR, FABP2, |
| | TTF2, PROS1, CTF1, SGCB, YME1L1, CAMP, |
| | ZC3H12A, AKR1B1, MMP7, AHR, CSF1, |
| | HDAC9, CTGF, KCNMA1, UGT1A, PRKCA, |
| | COMT, S100B, EGR1, PRL, IL15, DRD4, |
| | CAMK2G, SLC22A2, CCL11, PGF, THPO, GP6, |
| | TACR1, NTS, HNF1A, SST, KCDN1, |
| | LOC646627, TBXAS1, CUP2J2, TBXA2R, |
| | ADH1C, ALOX12, AHSG, BHMT, GJA4, |
| | SLC25A4, ACLY, ALOX5AP, NUMA1, |
| | CYP27B1, CYSLTR2, SOD3, LTC4S, UCN, |
| | GHRL, APOC2, CLEC4A, KBTBD10, TNC, |
| | TYMS, SHC1, LRP1, SOCS3, ADH1B, KLK3, |
| | HSD11B1, VKORC1, SERPINB2, TNS1, |
| | RNF19A, EPOR, ITGAM, PITX2, MAPK7, |
| | FCGR3A, LEEPR, ENG, GPX1, GOT2, HRH1, |
| | NR112, CRH, HTR1A, VDAC1, HPSE, SFTPD, |
| | TAP2, RMF123, PTK2Bm NTRK2, IL6R, ACHE, |
| | GLP1R, GHR, GSR, NQO1, NR5A1, GJB2, |
| | SLC9A1, MAOA, PCSK9, FCGR2A, SERPINF1, |
| | EDN3, UCP2, TFAP2A, C4BPA, SERPINF2, |
| | TYMP, ALPP, CXCR2, SLC3A3, ABCG2, ADA, |
| | JAK3, HSPA1A, FASN, FGF1, F11, ATP7A, |
| | CR1, GFPA, ROCK1, MECP2, MYLK, BCHE, |
| | LIPE, ADORA1, WRN, CXCR3, CD81, SMAD7, |
| | LAMC2, MAP3K5, CHGA, IAPP, RHO, ENPP1, |
| | PTHLH, NRG1, VEGFC, ENPEP, CEBPB, |
| | NAGLU, F2RL3, CX3CL1, BDKRB1, |
| | ADAMTS13, ELANE, ENPP2, CISH, GAST, |
| | MYOC, ATP1A2, NF1, GJB1, MEF2A, VCL, |
| | BMPR2, TUBB, CDC42, KRT18, HSF1, MYB, |
| | PRKAA2, ROCK2, TFP1, PRKG1, BMP2, |
| | CTNND1, CTH, CTSS, VAV2, NPY2R, IGFBP2, |
| | CD28, GSTA1, PPIA, APOH, S100A8, IL11, |
| | ALOX15, FBLN1, NR1H3, SCD, GIP, CHGB, |
| | PRKCB, SRD5A1, HSD11B2, CALCRL, |
| | GALNT2, ANGPTL4, KCNN4, PIK3C2A, |
| | HBEGF, CYP7A1, HLA-DRB5, BNIP3, GCKR, |
| | S100A12, PADI4, HSPA14, CXCR1, H19, |
| | KRTAP19-3, IDDM2, RAC2, YRY1, CLOCK, |
| | NGFR, DBH, CHRNA4, CACNA1C, PRKAG2, |
| | CHAT, PTGDS, NR1H2, TEK, VEGFB, MEF2C, |
| | MAPKAPK2, TNFRSF11A, HSPA9, CYSLTR1, |
| | MAT1A, OPRL1, IMPA1, CLCN2, |
| | DLD, PSMA6, PSMBS, CHI3L1, ALDH1B1, |
| | PARP2, STAR, LBP, ABCC6, RGS2, EFNB2, |
| | GJB6, APOA2, AMPD1, DYSF, FDFT1, EMD2, |
| | CCR6, GJB3, IL1RL1, ENTPD1, BBS4, |
| | CELSR2, F11R, RAPGEF3, HYAL1, ZNF259, |
| | ATOX1, ATF6, KHK, SAT1, GGH, TIMP4, |
| | SLC4A4, PDE2A, PDE3B, FADS1, FADS2, |
| | TMSB4X, TXNIP, LIMS1, RHOB, LY96, FOXO1, |
| | PNPLA2, TRH, GJC1, SLC7A5, FTO, GJD2, |
| | PRSC1, CASP12, GPBAR1, PXK, IL33, TRIB1, |
| | PBX4, NUPR1, 15-SEP, CILP2, TERC, GGT2, |
| | MTCO1, UOX, AVP, ANGPLT3 |
| Cataract | CRYAA, CRYA1, CRYBB2, CRYB2, PITX3, |
| | BFSP2, CP49, CP47, CRYAA, PAX6, AN2, |
| | MGDA, CRYBA1, CRYB1, CRYGC, CRYG3, |
| | CCL, LIM2, MP19, CRYGD, CRYG4, BFSP2, |
| | CP49, CP47, HSF4, CTM, MIP, AQPO, CRYAB, |
| | CRYA2, CTPP2, CRYBB1, CRYGD, CRYG4, |
| | CRYBB2, CRYB2, CCL, GJA8, CX50, CAE1, |
| | GJA3, CX46, CZP3, CAE3, CCM1, CAM, KRIT1 |
| Catecholaminergic Polymorphic | CASQ2 |
| Ventricular Tachycardia (CPVT) | |
| CDKL-5 Deficiencies or Mediated | CDKL5 |
| Diseases | |
| Charcot-Marie-Tooth (CMT) | PMP22 (CMT1A and E), MPZ (CMT1B), LITAF |
| disease (Types 1, 2, 3, and 4) | (CMT1C), EGR2 (CMT1D), NEFL (CMT1F), |
| | GJB1 (CMT1X), MFN2 (CMT2A), KIF1B |
| | (CMT2A2B), RAB7A (CMT2B), TRPV4 |
| | (CMT2C), GARS (CMT2D), NEFL (CMT2E), |
| | GAPD1 (CMT2K), HSPB8 (CMT2L), |
| | DYNC1H1 (CMT2O), LRSAM1 (CMT2P), |
| | IGHMBP2 (CMT2S), MORC2 (CMT2Z), |
| | GDAP1 (CMT4A), MTMR2 or SBF2/MTMR13 |
| | (CMT4B), SH3TC2 (CMT4C), NDRG1 |
| | (CMT4D), PRX (CMT4F), |
| | FIG4 (CMT4J), NT-3 |
| Chediak-Higashi Syndrome | LYST |
| Choroideremia | CHM, REP1 |
| Chorioretinal Atrophy | PRDM13, RGR, TEAD1 |
| Chronic Granulomatous Disease | CYBA, CYBB, NCF1, NCF2, NCF4 |
| Chronic Heart Failure | SERCA2 |
| Chronic Mucocutaneous | AIRE, CARD9, CLEC7A IL12B, IL12B1, IL1F, |
| Candidiasis | IL17RA, IL17RC, RORC, STAT1, STAT3, |
| | TRAF31P2 |
| Cirrhosis | KRT18, KRT8, CIRH1A, NAIC, TEX292, |
| | KIAA1988 |
| Colon Cancer (Familial | FAP: APC, HNPCC: MSH2, MLH1, PMS2, SH6, |
| Adenomatous Polyposis (FAP) | PMS1 |
| and Hereditary Nonpolyposis | |
| Colon Cancer (HNPCC)) | |
| Combined Immunodeficiency | IL2RG, SCIDX1, SCIDX, IMD4, HIV-1 (CCL5, |
| | SCYA5, D17S136E, TCP228) |
| Cone(-rod) Dystrophy | AIPL1, CRX, GUA1A, GUCY2D, PITPM3, |
| | PROM1, PRPH2, RIMS1, SEMA4A, ABCA4, |
| | ADAM9, ATF6, C21ORF2, C8ORF37, |
| | CACNA2D4, CDHR1, CERKL, CNGA3, CNGB3, |
| | CNNM4, CNAT2, IFT81, KCNV2, PDE6C, |
| | PDE6H, POC1B, RAX2, RDH5, RPGRIP1, |
| | TTLL5, RetCG1, GUCY2E |
| Congenital Stationary | CABP4, CACNA1F, CACNA2D4, GNAT1, |
| Night Blindness | CPR179, GRK1, GRM6, LRIT3, NYX, PDE6B, |
| | RDH5, RHO, RLBP1, RPE65, SAG, SLC24A1, |
| | TRPM1 |
| Congenital Fructose | ALDOB |
| Intolerance | |
| Cori's Disease (Glycogen | AGL |
| Storage Disease Type III) | |
| Corneal Clouding and Dystrophy | APOA1, TGFB1, CSD2, CDGG1, CSD, |
| | BIGH3, CDG2, TACSTD2, TROP2, M1S1, |
| | VSX1, RINX, PPCD, PPD, KTCN, COL8A2, |
| | FECD, PPCD2, PIP5K3, CFD |
| Cornea Plana Congenital | KERA, CNA2 |
| Cri Du Chat Syndrome, also | Deletions involving only band 5p15.2 to the |
| known as 5p Syndrome and | entire short arm of chromosome 5, e.g., |
| Cat Cry Syndrome | CTNND2, TERT |
| Crigler-Najjar syndrome | UGT1A1 |
| Cystic Fibrosis (CF) | CTFR, ABCC7, CF, MRP7, SCNN1A, those |
| | described in WO2015157070 |
| Diabetic Nephropathy | Gremlin, 12/15- lipoxygenase, TIM44 |
| Dent Disease (Types 1 and 2) | Type 1: CLCN5, Type 2: ORCL |
| Dentatorubral-Pallidoluysian | Atrophin-1 and ATN1 |
| Atrophy (DRPLA) (aka Haw | |
| River and Naito-Oyanagi | |
| Disease) | |
| Down Syndrome | Chromosome 21 trisomy |
| Drug Addiction | PRKCE, DRD2, DRD4, ABAT, GRIA2, GRM5, |
| | GRIN1, HTR1b, GRIN2A, DRD3, PDYN, GRIA1 |
| Duane Syndrome (Types 1, | CHN1, indels on chromosomes 4 and 8 |
| 2, and 3, including | |
| subgroups A, B, and C). | |
| Other names for this | |
| condition include Duane's | |
| Retraction Syndrome (or DR | |
| Syndrome), Eye Retraction | |
| Syndrome, Retraction | |
| Syndrome, Congenital | |
| retraction Syndrome, | |
| and Stilling-Turk-Duane | |
| Syndrome | |
| Duchenne Muscular Dystrophy | DMD, BMD, dystrophin gene, intron flanking |
| (DMD) | exon 51 of DMD gene, exon 51 mutations in |
| | DMD gene, see also WO2013163628 and U.S. |
| | Pat. Pub. 20130145487 |
| Eating Disorders | BDNF |
| Edward's Syndrome | Complete or partial trisomy of chromosome 18 |
| (Trisomy 18) | |
| Ehlers-Danlos Syndrome | COL5A1, COL5A2, COL1A1, COL3A1, TNXB, |
| (Types I-VI) | PLOD1, COL1A2, FKBP14, ADAMTS2 |
| Emery-Dreifuss Muscular | LMNA, LMN1, EMD2, FPLD, CMD1A, HGPS, |
| Dystrophy | LGMD1B, LMNA, LMN1, EMD2, FPLD, CMD1A |
| Enhanced S-Cone Syndrome | NR2E3, NRL |
| Epilepsy | NPY |
| Fabry's Disease | GLA, AGA |
| Facioscapulohumeral | FSHMD1A, FSHD1A, FRG1 |
| Muscular Dystrophy | |
| Factor H and Factor H-like 1 | HF1, CFH, HUS |
| Factor V Leiden Thrombophilia | Factor V (F5) |
| and Factor V Deficiency | |
| Factor V and Factor VII | MCFD2 |
| Deficiency | |
| Factor VII Deficiency | F7 |
| Factor X Deficiency | F10 |
| Factor XI Deficiency | F11 |
| Factor XII Deficiency | F12, HAF |
| Factor XIIIA Deficiency | F13A1, F13A |
| Factor XIIIB Deficiency | F13B |
| Familial Hypercholesterolemia | APOB, LDLR, PCSK9 |
| Familial Mediterranean Fever | MEFV |
| (FMF) also called Recurrent | |
| Polyserositis or Familial | |
| Paroxysmal Polyserositis | |
| Fanconi Anemia | FANCA, FACA, FA1, FA, FAA, FAAP95, |
| | FAAP90, FLJ34064, FANCC, FANCG, RAD51, |
| | BRCA1, BRCA2, BRIP1, BACH1, FANCJ, |
| | FANCB, FANCD1, FANCD2, FANCD, FAD, |
| | FANCE, FACE, FANCF, FANCI, ERCC4, |
| | FANCL, FANCM, PALB2, RAD51C, SLX4, |
| | UBE2T, FANCB, XRCC9, PHF9, KIAA1596 |
| Fanconi Syndrome Types I | FRTS1, GATM |
| (Childhood onset) and II | |
| (Adult Onset) | |
| Fragile X Syndrome and related | FMR1, FMR2, FXR1, FXR2, mGLUR5 |
| disorders | |
| Fragile XE Mental Retardation | FMR1 |
| (aka Martin Bell Syndrome) | |
| Friedreich Ataxia (FRDA) | FXN/X25 |
| Fuchs Endothelial Corneal | TCF4, COL8A2 |
| Dystrophy | |
| Galactosemia | GALT, GALK1, GALE |
| Gastrointestinal Epithelial | CISH |
| Cancer, GI cancer | |
| Gaucher Disease (Types 1, | GBA, GCase |
| 2, and 3, as well as other | |
| unusual forms that may not | |
| fit into these types) | |
| Glaucoma | MYOC, TIGR, GLC1A, JOAG, GPOA, OPTN, |
| | GLC1E, FIP2, HYPL, NRP, CYP1B1, GLC3A, |
| | OPA1, NTG, NPG, CYP1B1, GLC3A, those |
| | described in WO2015153780 |
| Glomerulosclerosis | CC chemokine ligand 2 |
| Glycogen Storage Diseases | SLC2A2, GLUT2, G6PC, G6PT, G6PT1, GAA, |
| (Types I-VI - See also | LAMP2, LAMPB, AGL, GDE, GBE1, GYS2, |
| Cori's Disease, Pompe | PYGL, PFKM, see also Cori's Disease, |
| Disease, McArdle's Disease, | Pompe's Disease, McArdle's disease, Hers |
| Hers Disease, and Von | Disease, and Von Gierke' s disease |
| Gierke's disease) | |
| RBC Glycolytic Enzyme | any mutations in a gene for an enzyme in the |
| Deficiency | glycolysis pathway including mutations in |
| | genes for hexokinases I and II, glucokinase, |
| | phosphoglucose isomerase, |
| | phosphofructokinase, aldolase Bm |
| | triosephosphate isomerase, glyceraldehyde-3- |
| | phosphate dehydrogenase, |
| | phosphoglycerokinase, phosphoglycerate |
| | mutase, enolase I, pyruvate kinase |
| GM2 Gangliosidoses (e.g., | Hexosaminidase-alpha, hexosaminidase-beta, |
| Sandhoff Disease and Tay-Sachs | HEX-A |
| Disease) | |
| Glycogen Storage Disease | G6Pase |
| Type Ia (GSDIa) | |
| Hemophilia (A or B) | F8, F9 |
| Hartnup Disease | SLC6A19 |
| Hearing Loss | NOX3, Hes5, BDNF |
| Hemochromatosis (HH) | HFE, H63D |
| Hemophagocytic | PRF1, HPLH2, UNC13D, MUNC13-4, HPLH3, |
| Lymphohistiocytosis Disorders | HLH3, FHL3 |
| Hemorrhagic Disorders | PI, ATT, F5 |
| Hers Disease (Glycogen | PYGL |
| Storage Disease Type VI) | |
| Hereditary Angioedema (HAE) | kalikrein B1 |
| Hereditary Hemorrhagic | ACVRL1, ENG, SMAD4 |
| Telangiectasia (Osler-Weber- | |
| Rendu Syndrome) | |
| Hereditary Spherocytosis | NK1, EPB42, SLC4A1, SPTA1, SPTB |
| Hereditary Persistence of | HBG1, HBG2, BCL11A, promoter region of |
| Fetal Hemoglobin | HBG 1 and/or 2 (in the CCAAT box) |
| Hemophilia (Hemophilia A | A: FVIII, F8C, HEMA |
| (Classic), B (aka Christmas | B: FVIX, HEME, FIX |
| disease), and C) | C: F9, F11 |
| Hepatic Adenoma | TCF1, HNF1A, MODY3 |
| Hepatic Failure, early | SCOD1, SCO1 |
| onset, and Neurologic | |
| Disorder | |
| Hepatic Lipase Deficiency | LIPC |
| Hepatoblastoma, Cancer, | CTNNB1, PDGFRL, PDGRL, PRLTS, AXIN1, |
| and Carcinomas | AXIN, CTNNB1, TP53, P53, LFS1, IGF2R, |
| | MPR1, MET, CASP8, MCH5 |
| Hermansky-Pudlak Syndrome | HPS1, HPS3, HPS4, HPS5, HPS6, HPS7, |
| | DTNBP1, BLOC1, BLOC1S2, BLOC3 |
| Human immunodeficiency | IL10, CSIF, CMKBR2, CCR2, CMKBR5, |
| viruses (HIV) susceptibility | CCCKR5 (CCR5), those in WO2015148670A1 |
| or infection | |
| Holoprosencephaly (HPE) | ACVRL1, ENG, SMAD4 |
| (Alobar, Semilobar, and | |
| Lobar) | |
| Homocystinuria | CBS, MTHFR, MTR, MTRR, MMADHC |
| Human papillomavirus (HPV) | HPV16, HPV18 E6/E7 |
| Herpes simplex virus | HSV1 genes (immediate early and late HSV-1 |
| (HSV)I, HSV2, and related | genes (UL1, 1.5, 5, 6, 8, 9, 12, 15, 16, 18, 19, |
| Keratitis | 22, 23, 26, 26.5, 27, 28, 29, 30, 31, 32, 33, 34, |
| | 35, 36, 37, 38, 42, 48, 49.5, 50, 52, 54, S6, |
| | RL2, RS1, those described in WO2015153789, |
| | WO2015153791 |
| Hunter's Syndrome (aka | IDS |
| Mucopolysaccharidosis | |
| Type II) | |
| Huntington's Disease | HD, HTT, IT15, PRNP, PRIP, JPH3, JP3, |
| (HD) and HD-like Disorders | HDL2, TBP, SCA17, PRKCE, BDNF, IGF1, |
| | EP300, RCOR1, PRKCZ, HDAC4, TGM2, |
| | those described in WO2013130824, |
| | WO2015089354 |
| Hurler's Syndrome (aka | IDUA, α-L-iduronidase |
| Mucopolysaccharidosis (MPS) | |
| Type IH, MPS IH) | |
| Hurler-Scheie Syndrome (aka | IDUA, α-L-iduronidase |
| Mucopolysaccharidosis | |
| Type IH-S, MPS IH-S) | |
| Hyaluronidase Deficiency | HYAL1 |
| (aka MPS IX) | |
| Hypercholesterolaemia or | PAH, LDLR |
| Phenylketonuria (PKU) | |
| Hyper IgM Syndrome | CD40L |
| Hypertension caused | Mineral corticoid receptor |
| Renal Damage | |
| Immunodeficiencies | CD3E, CD3G, AICDA, AID, HIGM2, TNFRSF5, |
| | CD40, UNG, DGU, HIGM4, TNFSF5, CD40LG, |
| | HIGM1, IGM, FOXP3, IPEX, AIID, XPID, PIDX, |
| | TNFRSF14B, TACI |
| Inborn errors of metabolism: | See also: Carbohydrate Metabolism Disorders |
| including urea cycle | (e.g., Galactosemia), Amino Acid Metabolism |
| disorders, organic acidemias, | Disorders (e.g., Phenylketonuria), Fatty Acid |
| fatty acid oxidation defects, | Metabolism (e.g., MCAD deficiency), Urea |
| amino acidopathies, | Cycle Disorders (e.g., Citrullinemia), Organic |
| carbohydrate disorders, | Acidemias (e.g., Maple Syrup Urine Disease), |
| mitochondrial disorders | Mitochondrial Disorders (e.g., MELAS), |
| | Peroxisomal Disorders (e.g., Zellweger |
| | syndrome) |
| Inflammation | IL-10, IL-1 (IL-1a, IL-1b), IL-13, IL-17 (IL-17a |
| | (CTLA8), IL-17b, IL-17c, IL-17d, IL-17f), II-23, |
| | Cx3crl, ptpn22, TNFa, NOD2/CARD15 for IBD, |
| | IL-6, IL-12 (IL-12a, IL-12b), CTLA4, Cx3c11 |
| Inflammatory Bowel Diseases | NOD2, IRGM, LRRK2, ATG5, ATG16L1, |
| (e.g., Ulcerative Colitis | IRGM, GATM, ECM1, CDH1, LAMB1, HNF4A, |
| and Cronh's Disease) | GNA12, IL10, CARD9/15, CCR6, IL2RA, |
| | MST1, TNFSF15, REL, STAT3, IL23R, IL12B, |
| | FUT2 |
| Interstitial Renal Fibrosis | TGF-β type II receptor |
| Job's Syndrome (aka | STAT3, DOCK8 |
| Hyper IgE Syndrome) | |
| Juvenile Retinoschisis | RS1, XLRS1 |
| Kabuki Syndrome 1 | MLL4, KMT2D |
| Kennedy Disease (aka | SBMA/SMAX1/AR |
| Spinobulbar Muscular | |
| Atrophy) | |
| Klinefelter Syndrome | Extra X chromosome in males |
| Lafora Disease | EMP2A, EMP2B |
| Leber Congenital | CRB1, RP12, CORD2, CRD, CRX, IMPDH1, |
| Amaurosis | OTX2, AIPL1, CABP4, CCT2, CEP290, |
| | CLUAP1, CRB1, CRX, DTHD1, GDF6, |
| | GUCY2D, IFT140, IQCB1, KCNJ13, LCA5, |
| | LRAT, NMNAT1, PRPH2, RD3, RDH12, |
| | RPE65, RP20, RPGRIP1, SPATA7, TULP1, |
| | LCA1, LCA4, GUC2D, CORD6, LCA3 |
| Leber Hereditary Optic | ND4 |
| Lesch-Nyhan Syndrome | HPRT1 |
| Leukocyte deficiencies | ITGB2, CD18, LCAMB, LAD, EIF2B1, EIF2BA, |
| and disorders | EIF2B2, EIF2B3, EIF2B5, LVWM, CACH, |
| | CLE, EIF2B4 |
| Leukemia | TAL1, TCL5, SCL, TAL2, FLT3, NBS1, NBS, |
| | ZNFN1A1, IK1, LYF1, HOXD4, HOX4B, BCR, |
| | CML, PHL, ALL, ARNT, KRAS2, RASK2, |
| | GMPS, AF10, ARHGEF12, LARG, KIAA0382, |
| | CALM, CLTH, CEBPA, CEBP, CHIC2, BTL, |
| | FLT3, KIT, PBT, LPP, NPM1, NUP214, |
| | D9S46E, CAN, CAIN, RUNX1, CBFA2, AML1, |
| | WHSC1L1, NSD3, FLT3, AF1Q, NPM1, |
| | NUMA1, ZNF145, PLZF, PML, MYL, STAT5B, |
| | AF10, CALM, CLTH, ARL11, ARLTS1, P2RX7, |
| | P2X7, BCR, CML, PHL, ALL, GRAF, NF1, |
| | VRNF, WSS, NFNS, PTPN11, PTP2C, SHP2, |
| | NS1, BCL2, CCND1, PRAD1, BCL1, TCRA, |
| | GATA1, GF1, ERYF1, NFE1, ABL1, NQO1, |
| | DIA4, NMOR1, |
| | NUP214, D9S46E, CAN, CAIN |
| Limb-Girdle Muscular | LGMD |
| Dystrophies | |
| Lipoprotein lipase | LPL |
| (LPL) deficiency | |
| Lowe Syndrome | OCRL |
| Lupus Glomerulonephritis | MAPK1 |
| Lysosomal storage disorders | GDNF |
| Machado-Joseph's Disease | ATX3 |
| (also known as | |
| Spinocerebellar Ataxia | |
| Type 3) | |
| Macular Degeneration | ABC4, CBC1, CHM1, APOE, C1QTNF5, C2, |
| | C3, CCL2, CCR2, CD36, CFB, CFH, CFHR1, |
| | CFHR3, CNGB3, CP, CRP, CST3, CTSD, |
| | CX3CR1, ELOVL4, ERCC6, FBLN5, FBLN6, |
| | FSCN2, HMCN1, HTRA1, IL6, IL8, PLEKHA1, |
| | PROM1, PRPH2, RPGR, SERPING1, TCOF1, |
| | TIMP3, TLR3 |
| Macular Dystrophy | BEST1, C1QTNF5, CTNNA1, EFEMP1, |
| | ELOVL4, FSCN2, GUCA1B, HMCN1, IMPG1, |
| | OTX2, PRDM13, PROM1, PRPH2, RP1L1, |
| | TIMP3, ABCA4, CFH, DRAM2, IMG1, MFSD8, |
| | ADMD, STGD2, STGD3, RDS, RP7, PRPH, |
| | AVMD, AOFMD, VMD2 |
| Malattia Leventinese | EFEMP1, FBLN3 |
| Malignant Melanoma | CD86, B7-2, IL-12 |
| Maple Syrup Urine | BCKDHA, BCKDHB, DBT |
| Disease | |
| Marfan Syndrome | FBN1 |
| Maroteaux-Lamy Syndrome | ARSB |
| (aka MPS VI) | |
| medium-chain acyl-CoA | MCAD |
| dehydrogenase (MCAD) | |
| deficiency | |
| McArdle's Disease | PYGM |
| (Glycogen Storage Disease | |
| Type V, myophosphorylase | |
| deficiency) | |
| Medullary Cystic Kidney | UMOD, HNFJ, FJHN, MCKD2, ADMCKD2 |
| Disease | |
| Metachromatic Leukodystrophy | ARSA |
| Methylmalonic Acidemia | MMAA, MMAB, MUT, MMACHC, MMADHC, |
| (MMA) | LMBRD1 |
| Mitochondrial Heteroplasmy, | APALI |
| Myoclonic Epilepsy with | |
| ragged red fibers (MERRF), | |
| or Mitochondrial | |
| Encephalomyopathy, Lactic | |
| Acidosis, and Stroke-Like | |
| Episodes (ME LAS) | |
| Morquio Syndrome (aka MPS | GALNS |
| IVA and B) | |
| Mucopolysaccharidosis Type | GUSB |
| VII (MPSVII) or Sly Syndrome | |
| Mucopolysaccharidosis | See also Hurler/Scheie Syndrome, Hurler |
| diseases (Types I H/S, I | Disease, Sanfilippo Syndrome, Scheie |
| H, II, III A B and C, I S, IVA | Syndrome, Morquio Syndrome, Hyaluronidase |
| and B, IX, VII, and VI) | Deficiency, Sly Syndrome, and Maroteaux- |
| | Lamy Syndrome |
| Muscular Atrophy | VAPB, VAPC, ALS8, SMN1, SMA1, SMA2, |
| | SMA3, SMA4, BSCL2, SPG17, GARS, |
| | SMAD1, CMT2D, HEXB, IGHMBP2, SMUBP2, |
| | CATF1, SMARD1 |
| Muscular Dystrophy | Sarcoglycan α, β, γ, Δ, ε, or ζ (SGCA, SGCB, |
| | SGCG, SGCD, SGCE, or SGCZ), FKRP, |
| | MDC1C, LGMD2I, LAMA2, LAMM, LARGE, |
| | KIAA0609, MDC1D, FCMD, TTID, MYOT, |
| | CAPN3, CANP3, DYSF, LGMD2B, LGMD2C, |
| | DMDA1, SCG3, ADL, DAG2, LGMD2D, |
| | DMDA2, LGMD2E, SGD, LGMD2F, CMD1L, |
| | TCAP, LGMD2G, CMD1N, TRIM32, HT2A, |
| | LGMD2H, FKRP, MDC1C, LGMD2I, TTN, |
| | CMD1G, TMD, LGMD2J, POMT1, CAV3, |
| | LGMD1C, SEPN1, SELN, RSMD1, PLEC1, |
| | PLTN, EBS1, dystrophin, mini-dystrophin |
| Myotonic Dystrophy (Type | CNBP (Type 2), DMPK (Type 1) |
| 1 and Type 2) | |
| Neoplasia | PTEN, ATM, ATR, EGFR, ERBB2, ERBB3, |
| | ERBB4, NOTCH1, NOTCH2, NOTCH3, |
| | NOTCH4, AKT, AKT2, AKT3, HIF, HIF1A, |
| | HIF3A, MET, HRG, BCL2, PPAR alpha, PPAR |
| | gamma, WT1 (Wilms Tumor), FGF Receptor |
| | Family members (5 members: 1, 2, 3, 4, 5), |
| | CDKN2A, APC, RB (retinoblastoma), MEN1, |
| | VHL, BRCA1, BRCA2, AR (Androgen |
| | Receptor), TSG101, IGF, IGF Receptor IGF1 |
| | (4 variants), IGF2 (3 variants), IGF1 Receptor, |
| | IGF 2 Receptor, BAX, BCL2, caspases family |
| | (9 members: 1, 2, 3, 4, 6, 7, 8, 9, 12), KRAS, |
| | APC |
| Neurofibromatosis (NF) | NF1, NF2 |
| (NF1, formerly | |
| Recklinghausen's NF, | |
| and NF2) | |
| Niemann-Pick Lipidosis | Types A and B: SMPD1, Type C: NPC1 or |
| (Types A, B, and C) | NPC2 |
| Noonan Syndrome | PTPN11, SOS1, RAF1, KRAS |
| Norrie Disease or | NDP |
| X-linked Familial Exudative | |
| Vitreoretinopathy | |
| North Carolina Macular | MCDR1 |
| Dystrophy | |
| Ornithine Carbamoyltransferase | OTC |
| Deficiency | |
| Osteogenesis Imperfecta (OI) | COL1A1, COL1A2, CRTAP, P3H |
| (Types I, II, III, IV, | |
| V, VI, and VII) | |
| Osteopetrosis | LRP5, BMND1, LRP7, LR3, OPPG, VBCH2, |
| | CLCN7, CLC7, OPTA2, OSTM1, GL, TCIRG1, |
| | TIRC7, OC116, OPTB1 |
| Patau's Syndrome (Trisomv 13) | Additional copy of chromosome 13 |
| Parkinson's Disease (PD) | SNCA (PARK1), UCHL1 (PARK 5), and |
| | LRRK2 (PARK8), PARK3, PARK2, PARK4, |
| | PARK7, |
| | PINKI (PARK6), x-Synuclein, DJ-1, Parkin, |
| | NR4A2, NURR1, NOT, TINUR, SNCAIP, TBP, |
| | SCA17, NCAP, PRKN, PDJ, DBH, NDUFV2, |
| | BDNF, GDNF, GBA, AADC, GAD1, GAD2, |
| | NRTN, NPY |
| Pattern Dystrophy of the | RDS/peripherin |
| retinal pigment epithelium | |
| (RPE) | |
| Phenylketonuria (PKU) | PAH, PKU1, QDPR, DHPR, PTS |
| Polycystic Kidney and | FCYT, PKHD1, ARPKD, PKD1, PKD2, PKD4, |
| Hepatic Disease | PKDTS, PRKCSH, G19P1, PCLD, SEC63 |
| Pompe Disease | GAA |
| Porphyria (actually refers | ALAD, ALAS2, CPOX, FECH, |
| to a group of different | HMBS, PPOX, UROD, UROS |
| diseases all having a | |
| specific heme production | |
| process abnormality) | |
| Posterior Polymorphous | TCF4, COL8A2 |
| Corneal Dystrophy | |
| Prader-Willi Syndrome | Deletion of region of short arm of |
| | chromosome 15, including UBE3A |
| Primary Hyperoxaluria | LDHA (lactate dehydrogenase A) and |
| (e.g., Type 1) | hydroxyacid oxidase 1 (HAO1) |
| Primary Open Angle | MYOC |
| Glaucoma (POAG) | |
| Primary Sclerosing | TCF4, COL8A2 |
| Cholangitis | |
| Progeria (also called | LMNA |
| Hutchinson-Gilford | |
| Progeria Syndrome) | |
| Progressive External | ANT-I |
| Ophthalmoplegia | |
| Propionic Acidaemias | PCCA |
| Prostate Cancer | HOXB13, MSMB, GPRC6A, TP53, GM-CSF |
| Pyruvate Dehydrogenase | PDHA1 |
| Deficiency | |
| Kidney/Renal Carcinoma | RLIP76, VEGF |
| Rett Syndrome | MECP2, RTT, PPMX, MRX16, MRX79, |
| | CDKL5, STK9, MECP2, RTT, PPMX, MRX16, |
| | MRX79, x- |
| | Synuclein, DJ-1 |
| Retinitis Pigmentosa (RP) | ADIPOR1, ABCA4, AGBL5, ARHGEF18, |
| | ARL2BP, ARL3, ARL6, BEST1, BBS1, BBS2, |
| | C2ORF71, C8ORF37, CA4, CERKL, CLRN1, |
| | CNGA1, CMGB1, CRB1, CRX, CYP4V2, |
| | DHDDS, DHX38, EMC1, EYS, FAM161A, |
| | FSCN2, GPR125, GUCA1B, HK1, HPRPF3, |
| | HGSNAT, IDH3B, IMPDH1, IMPG2, IFT140, |
| | IFTI72, KLHL7, KIAA1549, KIZ, LRAT, MAK, |
| | MERTK, MVK, NEK2, NUROD1, NR2E3, NRL, |
| | OFD1, PDE6A, PDE6B, PDE6G, POMGNT1, |
| | PRCD, PROM1, PRPF3, PRPF4, PRPF6, |
| | PRPF8, PRPF31, PRPH2, RPB3, RDH12, |
| | REEP6, RP39, RGR, RHO, RLBP1, ROM1, |
| | RP1, RP1L1, RPY, RP2, RP9, RPE65, RPGR, |
| | SAMD11, SAG, SEMA4A, SLC7A14, |
| | SNRNP200, SPP2, SPATA7, TRNT1, |
| | TOPORS, TTC8, TULP1, USH2A, ZNF408, |
| | ZNF513, see also 20120204282 |
| Sanfilippo Syndrome or | NAGLU |
| Mucopolysaccharidosis | |
| Type III B (MPS IIIB) | |
| short-chain acyl-CoA | SCAD |
| dehydrogenase (SCAD) | |
| deficiency | |
| Scheie Syndrome (also | IDUA, α-L-iduronidase |
| known as Mucopolysaccharidosis | |
| Type I S (MPS I-S)) | |
| Schizophrenia | Neuregulin1 (NRG1), ERB4 (receptor for |
| | Neuregulin), Complexin1 (CPLX1), TPH1 |
| | Tryptophan hydroxylase, TPH2 Tryptophan |
| | hydroxylase 2, Neurexin 1, GSK3, GSK3a, |
| | GSK3b, 5-HTT (Slc6a4), COMT, DRD (Drd1a), |
| | SLC6A3, DAOA, DTNBP1, Dao (Dao1), TCF4, |
| | COL8A2 |
| Secretase Related | APH-1 (alpha and beta), PSEN1, |
| Disorders | NCSTN, PEN-2, Nos1, Parp1, Nat1, Nat2, |
| | CTSB, APP, APH1B, PSEN2, PSENEN, |
| | BACE1, ITM2B, CTSD, NOTCH1, TNF, INS, |
| | DYT10, ADAM17, APOE, ACE, STN, TP53, |
| | IL6, NGFR, IL1B, ACHE, CTNNB1, IGF1, |
| | IFNG, NRG1, CASP3, MAPK1, CDH1, APBB1, |
| | HMGCR, CREB1, PTGS2, HES1, CAT, |
| | TGFB1, ENO2, ERBB4, TRAPPC10, MAOB, |
| | NGF, MMP12, JAG1, CD40LG, PPARG, |
| | FGF2, LRP1, NOTCH4, MAPK8, PREP, |
| | NOTCH3, PRNP, CTSG, EGF, REN, CD44, |
| | SELP, GHR, ADCYAP1, INSR, GFAP, MMP3, |
| | MAPK10, SP1, MYC, CTSE, PPARA, JUN, |
| | TIMP1, IL5, IL1A, MMP9, HTR4, HSPG2, |
| | KRAS, CYCS, SMG1, IL1R1, PROK1, MAPK3, |
| | NTRK1, IL13, MME, TKT, CXCR2, CHRM1, |
| | ATXN1, PAWR, NOTCJ2, M6PR, CYP46A1, |
| | CSNK1D, MAPK14, PRG2, PRKCA, L1 CAM, |
| | CD40, NR1I2, JAG2, CTNND1, CMA1, |
| | SORT1, DLK1, THEM4, JUP, CD46, CCL11, |
| | CAV3, RNASE3, HSPA8, CASP9, CYP3A4, |
| | CCR3, TFAP2A, SCP2, CDK4, JOF1A, |
| | TCF7L2, B3GALTL, MDM2, RELA, CASP7, |
| | IDE, FANP4, CASK, ADCYAP1R1, ATF4, |
| | PDGFA, C21ORF33, SCG5, RMF123, NKFB1, |
| | ERBB2, CAV1, MMP7, TGFA, RXRA, STX1A, |
| | PSMC4, P2RY2, TNFRSF21, DLG1, NUMBL, |
| | SPN, PLSCR1, UBQLN2, UBQLN1, PCSK7, |
| | SPON1, SILV, QPCT, HESS, GCC1 |
| Selective IgA Deficiency | Type 1: MSH5, Type 2: TNFRSF13B |
| Severe Combined | JAK3, JAKL, DCLRE1C, ARTEMIS, SCIDA, |
| Immunodeficiency (SCID), | RAG1, RAG2, ADA, PTPRC, CD45, LCA, |
| SCID-XI, and Adenosine | IL7R, CD3D, T3D, IL2RG, SCIDX1, SCIDX, |
| deaminase (ADA)-SCID | IMD4, those identified in U.S. Pat. App. Pub. |
| | 20110225664, 20110091441, 20100229252, |
| | 20090271881 and 20090222937 |
| Sickle Cell Disease | HBB, BCL11A, BCL11Ae, cis- regulatory |
| | elements of the B-globin locus, HBG 1/2 |
| | promoter, HBG distal CCAAT box region |
| | between −92 and −130 of the HBG |
| | Transcription Start Site, those described in |
| | WO2015148863, WO 2013/126794, |
| | U.S. Pat. Pub. 20110182867 |
| Spinocerebellar Ataxias | ATXN1, ATXN2, ATX3 |
| (SCA Types 1, 2, 3, 6, | |
| 7, 8, 12, and 17) | |
| Sorsby Fundus Dystrophy | TIMP3 |
| Stargardt Disease | ABCR, ELOVL4, ABCA4, PROM1 |
| Thalassemia (Alpha, | HBA1, HBA2 (Alpha), HBB (Beta), HBB and |
| Beta, Delta) | HBD (delta), LCRB, BCL11A, BCL11Ae, cis- |
| | regulatory elements of the B-globin locus, HBG |
| | 1/2 promoter, those described in |
| | WO2015148860, U.S. Pat. Pub. |
| | 20110182867, 2015/148860 |
| Thymic Aplasia (DiGeorge | Deletion of 30 to 40 genes in the middle of |
| Syndrome; 22q 11.2 | chromosome 22 at a location known as |
| Deletion Syndrome) | 22g11.2, including TBX1, DGCR8 |
| Transthyretin Amyloidosis | TTR (transthyretin) |
| (ATTR) | |
| Trimethylaminuria | FMO3 |
| Trinucleotide Repeat | HTT, SBMA/SMAX1/AR, FXN/X25 ATX3, |
| Disorders (generally) | ATXN1, ATXN2, DMPK, Atrophin-1 and Atn1 |
| | (DRPLA Dx), CBP (Creb-BP - global |
| | instability), VLDLR, Atxn7, Atxn10, FEN1, |
| | TNRC6A, PABPN1, JPH3, MED15, ATXN3, |
| | TBP, CACNA1A, ATXN80S, PPP2R2B, |
| | ATXN7, TNRC6B, TNRC6C, CELF3, |
| | MAB21L1, MSH2, TMEM185A, SIX5, CNPY3, |
| | RAXE, GNB2, RPL14, ATXN8, ISR, TTR, |
| | EP400, GIGYF2, OGG1, STC1, CNDP1, |
| | C10ORF2, MAML3, DKC1, PAXIP1, CASK, |
| | MAPT, SP1, POLG, AFF2, THBS1, TP53, |
| | ESR1, CGGBP1, ABT1, KLK3, PRNP, JUN, |
| | KCNN3, BAX, FRAXA, KBTBD10, MBNL1, |
| | RAD51, NCOA3, ERDA1, TSC1, COMP, |
| | GGLC, RRAD, MSH3, DRD2, CD44, CTCF, |
| | CCND1, CLSPN, MEF2A, PTPRU, GAPDH, |
| | TRIM22, WT1, AHR, GPX1, TPMT, NDP, |
| | ARX, TYR, EGR1, UNG, NUMBL, FABP2, |
| | EN2, CRYGC, SRP14, CRYGB, PDCD1, |
| | HOXA1, ATXN2L, PMS2, GLA, CBL, FTH1, |
| | IL12RB2, OTX2, HOXA5, POLG2, DLX2, |
| | AHRR, MANF, RMEM158, see also |
| | 20110016540 |
| Triple X (XXX) Syndrome | X chromosome trisomy |
| Turner's Syndrome (XO) | Monosomy X |
| Tuberous Sclerosis | TSC1, TSC2 |
| Usher Syndrome (Types I, | ABHD12, CDH23, CIB2, CLRN1, DFNB31, |
| II, and III) | GPR98, HARS, MYO7A, PCDH15, USH1C, |
| | USH1G, USH2A, USHI11A, those described in |
| | WO2015134812A1 |
| Velocardiofacial Syndrome | Many genes are deleted, COM, TBX1, and |
| (aka 22q11.2 Deletion | other are associated with symptoms |
| Syndrome, DiGeorge Syndrome, | |
| Conotruncal Anomaly Face | |
| Syndrome (CTAF), Autosomal | |
| Dominant Opitz G/BB | |
| Syndrome or Cayler | |
| Cardio-Facial Syndrome) | |
| very long-chain acyl-CoA | VLCAD |
| dehydrogenase (VLCAD) | |
| deficiency | |
| Von Gierke's Disease | G6PC, SLC37A4 |
| (Glycogen Storage | |
| Disease Type I) | |
| Von Hippel-Lindau Syndrome | VHL |
| Von Willebrand Disease | VWF |
| (Types I, II, and III) | |
| Wilson Disease | ATP7B |
| Wiskott-Aldrich Syndrome | WAS |
| Xeroderma Pigmentosum | POLH |
| X-linked myotubular myopathy | MTM1 |

ii. Administering a Codon Optimized Gene Product

The method of treatment includes delivering a codon optimized gene product or a pharmaceutical composition containing the same to the subject (e.g., a human) by any appropriate route of administration (e.g., intramuscular, subcutaneous, intradermal, intravenous, intraperitoneal, intraarterial, intraventricular, intranasal, intraorbital, intracranial, or intraosseous administration). In some embodiments, the method of treatment includes administering one codon optimized gene product or a pharmaceutical composition containing the same to a subject to produce a therapeutic effect. In some embodiments, the codon optimized gene product or a pharmaceutical composition containing the same is administered to a subject as a monotherapy. In some embodiments, the codon optimized gene product or a pharmaceutical composition containing the same is administered to the subject with one or more additional therapies (e.g., 1, 2, 3, 4, or 5 additional therapeutic agents) as a combination therapy. In some embodiments, the combination therapy includes delivering two or more (e.g., 2, 3, 4, 5, or more than 5) codon optimized gene products or pharmaceutical compositions containing the same to a subject. In some embodiments, the combination therapy includes delivering one or more codon optimized gene products or pharmaceutical compositions containing the same to the subject with one or more additional therapeutic agents or other standard of care interventions for a particular disease or condition. In some embodiments, the combination therapy is administered to the subject to produce an additive or synergistic therapeutic effect.

The codon optimized gene product or a pharmaceutical composition containing the same may be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. In some embodiments, treatment is used to reduce, slow, and/or inhibit the onset or progression of a disease or condition. In other embodiments, the methods may be used to alleviate, ameliorate, reduce, or reverse clinical manifestations of a disease or condition. In some embodiments, the method of treatment includes administering one or more codon optimized gene products to a subject in a single dose. In some embodiments, the method of treatment includes administering one or more codon optimized gene products to a subject in multiple doses. Administration may occur any suitable number of times per day, week, month or year, and for as long as necessary. Subjects may be adult or pediatric humans, with or without a comorbid condition.

iii. Evaluating Efficacy

Determining the efficacy of a method of treatment described herein may require evaluating the subject's response to the treatment. The subject's response may be evaluated in an in-patient treatment setting or an out-patient treatment setting. Evaluation of the subject's response may occur one or more times following the administration of one or more codon optimized gene products or a pharmaceutical composition containing the same. Evaluation of the subject's response may occur continuously, either sporadically or at designated time points following administration of the one or more codon optimized gene products. Evaluation of the subject's response may occur, for example, 1-10 days after administration of the one or more codon optimized gene product (e.g., 7-10 days, 6-8 days, 5-7 days, 3-5 days, 2-4 days, 1-3 days, or within 1 day following administration of the one or more codon optimized gene product; e.g., 10 days, 9 days, 8 days, 7 days, 6 days, 5 days, 4 days, 3 days, 2 days, 1 day, or less than a day following administration of the one or more codon optimized gene product). Evaluation of the subject's response may occur 1-12 weeks or more after administration of the one or more codon optimized gene product (e.g., 10-12 weeks, 8-12 weeks, 6-12 weeks, 8-10 weeks, 6-10 weeks, 4-10 weeks, 6-8 weeks, 4-8 weeks, 2-8 weeks, 4-6 weeks, 2-6 weeks, 3-6 weeks, 2-4 weeks, 1-3 weeks, or 1-2 weeks following administration of the one or more codon optimized gene product; e.g., later than 12 weeks, 12 weeks, 11 weeks, 10 weeks, 9 weeks, 8 weeks, 7 weeks, 6 weeks, 5 weeks, 4 weeks, 3 weeks, 2 weeks, or 1 week following administration of the one or more codon optimized gene product). Evaluation of the subject's response may occur 1-12 months after administration of the one or more codon optimized gene product (e.g., 10-12 months, 8-months, 6-8 months, 4-6 months, 3-5 months, 2-4 months, or 1-3 months following administration 10 of the one or more codon optimized gene product; e.g., 12 months, 11 months, 10 months, 9 months, 8 months, 7 months, 6 months, 5 months, 4 months, 3 months, 2 months, or 1 month following administration of the one or more codon optimized gene product). Evaluation of the subject's response may occur 1-5 years or longer after administration of the one or more codon optimized gene product (e.g., 4-5 years, 3-5 years, 2-3 years, or 1-3 years following administration of the one or more codon optimized gene product; e.g., later than 5 years, 5 years, 4 years, 3 years, 2 years, or 1 year following administration of the one or more codon optimized gene product).

The efficacy of the method of treatment may be evaluated by comparing one or more metrics of a subject that is administered the treatment compared to a relevant control or reference. In some embodiments, the efficacy of the method of treatment is evaluated by comparing one or more metrics of a subject undergoing treatment to one or more metrics of the same subject prior to treatment. In some embodiments, the efficacy of the method of treatment is evaluated by comparing one or more metrics of a subject undergoing treatment to one or more metrics of a different subject who has the same disease or condition and has not undergone treatment. In further embodiments, the efficacy of the method of treatment is evaluated by comparing one or more metrics of a subject undergoing treatment to one or more metrics of a healthy subject (e.g., one who does not have the disease or condition).

In some embodiments, the metric is the expression level of the protein or polypeptide encoded by a codon optimized gene product or an mRNA transcript thereof. In some embodiments, the metric is the activity or biological function of a protein or polypeptide, in which the protein is the same as the one encoded by the codon optimized gene product, or a different protein (e.g., a downstream protein, an effector protein, or a binding protein). In some embodiments, the expression or activity of one or more proteins of interest is evaluated by taking one or more biological samples from the subject and comparing the results from earlier measurements from the subject and/or results from the subject that were measured prior to administration of the one or more codon optimized gene product. In some embodiments, the expression or activity of the one or more proteins of interest is detected by biological or analytical methods known in the art or described herein.

Depending on the efficacy of the treatment administered to the subject, the treatment regimen may change over the course of treatment. The treatment regimen and efficacy thereof may be determined by a practitioner skilled in the art (e.g., a physician, clinician, or medical specialist). In some embodiments, the dose of the one or more codon optimized gene product or a pharmaceutical composition containing the same may be adjusted (e.g., decreased or increased) over the course of treatment. In some embodiments, the dose of the one or more codon optimized gene product or a pharmaceutical composition containing the same may be increased by about 10%, 25%, 50%, 75%, 100%, 125%, 150%, 175%, 200%, or more over the course of treatment. In some embodiments, the dose of the one or more codon optimized gene product or a pharmaceutical composition may be decreased by about 10%, 25%, 50%, 75%, or more than 75% over the course of treatment. In some embodiments, the frequency of administration of the one or more codon optimized gene product or a pharmaceutical composition may increase or decrease over the course of treatment. In further embodiments, one or more additional therapies may be further added to the treatment regimen depending on the disease or condition and the standard of care thereof.

### III. Kits

The compositions or methods described herein can be provided in a kit for use in producing a protein or gene product. In some embodiments, the compositions and methods described herein can be provided in a kit for use in treating a disease or condition. In some embodiments, the kit may include a package insert that instructs a user of the kit to perform any one of the methods of codon optimization described herein. In further embodiments, the kit may include a package insert that instructs a user of the kit to perform any one of the methods of treatment described herein. The kit may optionally include a syringe or device for administering the compositions of the present disclosure. In some embodiments, the kit may include one or more additional therapeutic agents. In some embodiments, the kit includes one or more antibodies or binding molecules to detect the expression or activity of a protein of interest.

### EXAMPLES

### Example 1. Determining ZsGreen Fluorescent Protein Yield Following 4 Distinct Codon Optimization Methods

This example describes the process of codon optimizing a sequence encoding a ZsGreen fluorescent protein reporter via two common codon usage schemes and comparatively analyzing the resulting sequences and protein production thereof with or without applying a series of codon usage rules to reduce or eliminate DRACH motifs. The two common codon usage schemes are the “all-best” (also called “AllBest” in this example) scheme, which refers to replacing one or more codons with the synonymous codon having the highest usage frequency, and the “proportional usage” scheme (also called “AllProb” in this example), which refers to modifying the gene or codon sequence such that one or more codons are represented proportional to their reported usage frequency. Exemplary codons and their usage frequency are shown in Table 1. The codon usage rules (also called “pRules” in this example) are a strategy for reducing m6A modifications through the removal of DRACH motifs and, in this and the following examples, are applied after modifying the coding sequence with one of the described codon usage schemes. The codon usage rules, or pRules, are as follows:
1. Never use GAC for aspartic acid (D)
2. Never use GAA for glutamic acid (E)
3. Never use GGA for glycine (G)
4. Never use AAA for lysine (K)
5. Never use AAC for asparagine (N)
6. Always use ACG for threonine (T)
   In this example, when the six codon usage rules were used, they were applied at every instance D, E, G, K, N, and T codons appear in the coding sequence. This approach eliminated all the DRACH motifs and changed many codons that were otherwise not implicated in m6A modifications.

The open reading frame (ORF) of the ZsGreen DNA sequence following the application of one or more codon optimization strategy to eliminate DRACH motifs are shown below:

1. ZsG ORF following codon optimization with the all-best codon usage scheme
>ZsG AllBest
(SEQ ID NO: 1)
ATGGCCCAGAGCAAGCACGGCCTGACCAAGGAGATGACCATGAAGTACCGGATGGAGGGCTGCGTGGACGGCCAC
AAGTTCGTGATCACCGGCGAGGGCATCGGCTACCCCTTCAAGGGCAAGCAGGCCATCAACCTGTGCGTGGTGGAG
GGCGGCCCCCTGCCCTTCGCCGAGGACATCCTGAGCGCCGCCTTCATGTACGGCAACCGGGTGTTCACCGAGTAC
CCCCAGGACATCGTGGACTACTTCAAGAACAGCTGCCCCGCCGGCTACACCTGGGACCGGAGCTTCCTGTTCGAG
GACGGCGCCGTGTGCATCTGCAACGCCGACATCACCGTGAGCGTGGAGGAGAACTGCATGTACCACGAGAGCAAG
TTCTACGGCGTGAACTTCCCCGCCGACGGCCCCGTGATGAAGAAGATGACCGACAACTGGGAGCCCAGCTGCGAG
AAGATCATCCCCGTGCCCAAGCAGGGCATCCTGAAGGGCGACGTGAGCATGTACCTGCTGCTGAAGGACGGCGGC
CGGCTGCGGTGCCAGTTCGACACCGTGTACAAGGCCAAGAGCGTGCCCCGGAAGATGCCCGACTGGCACTTCATC
CAGCACAAGCTGACCCGGGAGGACCGGAGCGACGCCAAGAACCAGAAGTGGCACCTGACCGAGCACGCCATCGCC
AGCGGCAGCGCCCTGCCCTGA
2. ZsG ORF following codon optimization with the all-best codon usage scheme
and then the six codon usage rules
>ZsG_ABpRules
(SEQ ID NO: 2)
ATGGCCCAGAGCAAGCACGGCCTGACGAAGGAGATGACGATGAAGTACCGGATGGAGGGCTGCGTGGATGGCCAC
AAGTTCGTGATCACGGGCGAGGGCATCGGCTACCCCTTCAAGGGCAAGCAGGCCATCAATCTGTGCGTGGTGGAG
GGCGGCCCCCTGCCCTTCGCCGAGGATATCCTGAGCGCCGCCTTCATGTACGGCAATCGGGTGTTCACGGAGTAC
CCCCAGGATATCGTGGATTACTTCAAGAATAGCTGCCCCGCCGGCTACACGTGGGATCGGAGCTTCCTGTTCGAG
GATGGCGCCGTGTGCATCTGCAATGCCGATATCACGGTGAGCGTGGAGGAGAATTGCATGTACCACGAGAGCAAG
TTCTACGGCGTGAATTTCCCCGCCGATGGCCCCGTGATGAAGAAGATGACGGATAATTGGGAGCCCAGCTGCGAG
AAGATCATCCCCGTGCCCAAGCAGGGCATCCTGAAGGGCGATGTGAGCATGTACCTGCTGCTGAAGGATGGCGGC
CGGCTGCGGTGCCAGTTCGATACGGTGTACAAGGCCAAGAGCGTGCCCCGGAAGATGCCCGATTGGCACTTCATC
CAGCACAAGCTGACGCGGGAGGATCGGAGCGATGCCAAGAATCAGAAGTGGCACCTGACGGAGCACGCCATCGCC
AGCGGCAGCGCCCTGCCCTGA
3. ZsG ORF following codon optimization with the proportional usage codon
usage scheme
>ZsG_AllProb
(SEQ ID NO: 3)
ATGGCCCAGTCAAAACATGGACTCACTAAAGAGATGACCATGAAATACAGAATGGAGGGGTGCGTCGATGGTCAC
AAATTCGTCATCACCGGCGAGGGGATTGGATATCCATTCAAGGGAAAGCAGGCCATTAACCTGTGCGTCGTGGAA
GGAGGACCACTGCCTTTCGCAGAAGATATCCTCAGTGCCGCATTCATGTATGGAAACAGGGTCTTCACTGAGTAC
CCACAGGATATTGTTGACTATTTTAAAAATTCTTGCCCAGCCGGCTATACATGGGACCGCTCCTTCCTCTTTGAA
GACGGAGCGGTGTGTATCTGCAACGCAGATATCACAGTGTCCGTTGAGGAAAACTGCATGTATCACGAAAGCAAG
TTCTATGGCGTGAATTTCCCTGCGGACGGGCCCGTTATGAAGAAGATGACAGACAATTGGGAGCCCAGTTGCGAG
AAAATCATTCCAGTGCCCAAGCAGGGGATTCTCAAGGGCGACGTAAGCATGTACCTGCTGCTAAAGGATGGAGGT
CGCTTACGCTGCCAGTTTGACACCGTCTATAAAGCCAAGAGCGTCCCCCGGAAGATGCCGGACTGGCATTTTATC
CAGCACAAGCTGACCCGTGAGGACAGGTCTGATGCCAAAAATCAGAAGTGGCACTTAACTGAGCATGCTATCGCA
AGCGGATCAGCACTCCCGTGA
4. ZsG ORF following codon optimization with the proportional usage codon
usage scheme and then the six codon usage rules
>ZsG_APpRules
(SEQ ID NO: 4)
ATGGCCCAGTCAAAGCATGGCCTCACGAAGGAGATGACGATGAAGTACAGAATGGAGGGGTGCGTCGATGGTCAC
AAGTTCGTCATCACGGGCGAGGGGATTGGGTATCCATTCAAGGGCAAGCAGGCCATTAATCTGTGCGTCGTGGAG
GGCGGCCCACTGCCTTTCGCAGAGGATATCCTCAGTGCCGCATTCATGTATGGCAATAGGGTCTTCACGGAGTAC
CCACAGGATATTGTTGATTATTTTAAGAATTCTTGCCCAGCCGGCTATACGTGGGATCGCTCCTTCCTCTTTGAG
GATGGGGCGGTGTGTATCTGCAATGCAGATATCACGGTGTCCGTTGAGGAGAATTGCATGTATCACGAGAGCAAG
TTCTATGGCGTGAATTTCCCTGCGGATGGGCCCGTTATGAAGAAGATGACGGATAATTGGGAGCCCAGTTGCGAG
AAGATCATTCCAGTGCCCAAGCAGGGGATTCTCAAGGGCGATGTAAGCATGTACCTGCTGCTAAAGGATGGCGGT
CGCTTACGCTGCCAGTTTGATACGGTCTATAAGGCCAAGAGCGTCCCCCGGAAGATGCCGGATTGGCATTTTATC
CAGCACAAGCTGACGCGTGAGGATAGGTCTGATGCCAAGAATCAGAAGTGGCACTTAACGGAGCATGCTATCGCA
AGCGGTTCAGCACTCCCGTGA

Following the codon optimization strategies, the number of DRACH motifs in the ZsGreen open reading frame were quantified, the results of which are shown in Table 3 below.

TABLE 3
Number of DRACH motifs following codon
optimization of the ZsGreen ORF

| | SEQ | Number of |
|---|---|---|
| Codon Optimization Strategy | ID NO: | DRACH motifs |
| All-best | 1 | 14 |
| All-best + 6 codon usage rules | 2 | 0 |
| Proportional usage | 3 | 16 |
| Proportional usage + 6 codon usage rules | 4 | 0 |

Following quantification of the number of DRACH motifs, the resulting ORFs were then aligned and compared to assess the differences among the codon optimization strategies. The comparisons are shown below, in which identical nucleotides are denoted with an asterisk (*), and differences in the polynucleotide sequence are shown in bold and underline.

1. Alignment of ZsGreen ORF following all-best codon usage scheme without (SEQ ID NO: 1) and with (SEQ ID NO: 2) application of the six codon usage rules:

| | |
|---|---|
| ZsG_AllBest | ATGGCCCAGAGCAAGCACGGCCTGACCAAGGAGATGACCATGAAGTACCGGATGGAGGGC 60 |
| ZsG_ABpRules | ATGGCCCAGAGCAAGCACGGCCTGACGAAGGAGATGACGATGAAGTACCGGATGGAGGGC 60 |
| | ************************** *********** ********************* |
| ZsG_AllBest | TGCGTGGACGGCCACAAGTTCGTGATCACCGGCGAGGGCATCGGCTACCCCTTCAAGGGC 120 |
| ZSG_ABpRules | TGCGTGGATGGCCACAAGTTCGTGATCACGGGCGAGGGCATCGGCTACCCCTTCAAGGGC 120 |
| | ******** ******************** ****************************** |
| ZsG_AllBest | AAGCAGGCCATCAACCTGTGCGTGGTGGAGGGCGGCCCCCTGCCCTTCGCCGAGGACATC 180 |
| ZsG_ABpRules | AAGCAGGCCATCAATCTGTGCGTGGTGGAGGGCGGCCCCCTGCCCTTCGCCGAGGATATC 180 |
| | ************** ***************************************** *** |
| ZsG_AllBest | CTGAGCGCCGCCTTCATGTACGGCAACCGGGTGTTCACCGAGTACCCCCAGGACATCGTG 240 |
| ZsG_ABpRules | CTGAGCGCCGCCTTCATGTACGGCAATCGGGTGTTCACGGAGTACCCCCAGGATATCGTG 240 |
| | ************************** *********** ************** ****** |
| ZsG_AllBest | GACTACTTCAAGAACAGCTGCCCCGCCGGCTACACCTGGGACCGGAGCTTCCTGTTCGAG 300 |
| ZsG_ABpRules | GATTACTTCAAGAATAGCTGCCCCGCCGGCTACACGTGGGATCGGAGCTTCCTGTTCGAG 300 |
| | ** *********** ******************** ***** ****************** |
| ZsG_AllBest | GACGGCGCCGTGTGCATCTGCAACGCCGACATCACCGTGAGCGTGGAGGAGAACTGCATG 360 |
| ZsG_ABpRules | GATGGCGCCGTGTGCATCTGCAATGCCGATATCACGGTGAGCGTGGAGGAGAATTGCATG 360 |
| | ** ******************** *********** ***************** ****** |
| ZsG_AllBest | TACCACGAGAGCAAGTTCTACGGCGTGAACTTCCCCGCCGACGGCCCCGTGATGAAGAAG 420 |
| ZsG_ABpRules | TACCACGAGAGCAAGTTCTACGGCGTGAATTTCCCCGCCGATGGCCCCGTGATGAAGAAG 420 |
| | ***************************** *********** ****************** |
| ZSG_AllBest | ATGACCGACAACTGGGAGCCCAGCTGCGAGAAGATCATCCCCGTGCCCAAGCAGGGCATC 480 |
| ZsG_ABpRules | ATGACGGATAATTGGGAGCCCAGCTGCGAGAAGATCATCCCCGTGCCCAAGCAGGGCATC 480 |
| | ***** ** ** ************************************************ |
| ZsG_AllBest | CTGAAGGGCGACGTGAGCATGTACCTGCTGCTGAAGGACGGCGGCCGGCTGCGGTGCCAG 540 |
| ZsG_ABpRules | CTGAAGGGCGATGTGAGCATGTACCTGCTGCTGAAGGATGGCGGCCGGCTGCGGTGCCAG 540 |
| | *********** ************************** ********************* |
| ZsG_AllBest | TTCGACACCGTGTACAAGGCCAAGAGCGTGCCCCGGAAGATGCCCGACTGGCACTTCATC 600 |
| ZsG_ABpRules | TTCGATACGGTGTACAAGGCCAAGAGCGTGCCCCGGAAGATGCCCGATTGGCACTTCATC 600 |
| | ***** ** ************************************** ************ |
| ZSG_AllBest | CAGCACAAGCTGACCCGGGAGGACCGGAGCGACGCCAAGAACCAGAAGTGGCACCTGACC 660 |
| ZSG_ABpRules | CAGCACAAGCTGACGCGGGAGGATCGGAGCGATGCCAAGAATCAGAAGTGGCACCTGACG 660 |
| | ************** ******** ******** ******** ****************** |
| ZsG_AllBest | GAGCACGCCATCGCCAGCGGCAGCGCCCTGCCCTGA 696 |
| ZsG_ABpRules | GAGCACGCCATCGCCAGCGGCAGCGCCCTGCCCTGA 696 |
| | ************************************ |

2. Alignment of ZsGreen ORF following proportional codon usage scheme without (SEQ ID NO: 3) and with (SEQ ID NO: 4) application of the six codon usage rules

| | |
|---|---|
| ZsG_AllProb | ATGGCCCAGTCAAAACATGGACTCACTAAAGAGATGACCATGAAATACAGAATGGAGGGG 60 |
| ZsG_APpRules | ATGGCCCAGTCAAAGCATGGCCTCACGAAGGAGATGACGATGAAGTACAGAATGGAGGGG 60 |
| | ************** ***** ***** ** ******** ***** *************** |
| ZsG_AllProb | TGCGTCGATGGTCACAAATTCGTCATCACCGGCGAGGGGATTGGATATCCATTCAAGGGA 120 |
| ZSG_APpRules | TGCGTCGATGGTCACAAGTTCGTCATCACGGGCGAGGGGATTGGGTATCCATTCAAGGGC 120 |
| | ***************** *********** ************** ************** |
| ZsG_AllProb | AAGCAGGCCATTAACCTGTGCGTCGTGGAAGGAGGACCACTGCCTTTCGCAGAAGATATC 180 |
| ZsG_APpRules | AAGCAGGCCATTAATCTGTGCGTCGTGGAGGGCGGCCCACTGCCTTTCGCAGAGGATATC 180 |
| | ************** ************** ** ** ***************** ****** |
| ZsG AllProb | CTCAGTGCCGCATTCATGTATGGAAACAGGGTCTTCACTGAGTACCCACAGGATATTGTT 240 |
| ZsG_APpRules | CTCAGTGCCGCATTCATGTATGGCAATAGGGTCTTCACGGAGTACCCACAGGATATTGTT 240 |
| | *********************** ** *********** ********************* |
| ZsG_AllProb | GACTATTTTAAAAATTCTTGCCCAGCCGGCTATACATGGGACCGCTCCTTCCTCTTTGAA 300 |
| ZsG_APpRules | GATTATTTTAAGAATTCTTGCCCAGCCGGCTATACGTGGGATCGCTCCTTCCTCTTTGAG 300 |
| | ** ******** *********************** ***** ****************** |
| ZsG AllProb | GACGGAGCGGTGTGTATCTGCAACGCAGATATCACAGTGTCCGTTGAGGAAAACTGCATG 360 |
| ZsG_APpRules | GAtGGGGCGGTGTGTATCTGCAATGCAGATATCACGGTGTCCGTTGAGGAgAATTGCATG 360 |
| | ** ** ***************** *********** ************** ** ****** |
| ZSG_AllProb | TATCACGAAAGCAAGTTCTATGGCGTGAATTTCCCTGCGGACGGGCCCGTTATGAAGAAG 420 |
| ZsG_APpRules | TATCACGAGAGCAAGTTCTATGGCGTGAATTTCCCTGCGGATGGGCCCGTTATGAAGAAG 420 |
| | ******** ******************************** ****************** |
| ZsG_AllProb | ATGACAGACAATTGGGAGCCCAGTTGCGAGAAAATCATTCCAGTGCCCAAGCAGGGGATT 480 |
| ZsG_APpRules | ATGACGGATAATTGGGAGCCCAGTTGCGAGAAGATCATTCCAGTGCCCAAGCAGGGGATT 480 |
| | ***** ** *********************** *************************** |
| ZsG_AllProb | CTCAAGGGCGACGTAAGCATGTACCTGCTGCTAAAGGATGGAGGTCGCTTACGCTGCCAG 540 |
| ZsG_APpRules | CTCAAGGGCGATGTAAGCATGTACCTGCTGCTAAAGGATGGCGGTCGCTTACGCTGCCAG 540 |
| | *********** ***************************** ****************** |
| ZSG AllProb | TTTGACACCGTCTATAAAGCCAAGAGCGTCCCCCGGAAGATGCCGGACTGGCATTTTATC 600 |
| ZsGAPpRules | TTTGATACGGTCTATAAGGCCAAGAGCGTCCCCCGGAAGATGCCGGATTGGCATTTTATC 600 |
| | ***** ** ******** ***************************** ************ |
| ZsG AllProb | CAGCACAAGCTGACCCGTGAGGACAGGTCTGATGCCAAAAATCAGAAGTGGCACTTAACT 660 |
| ZsG_APpRules | CAGCACAAGCTGACGCGTGAGGATAGGTCTGATGCCAAGAATCAGAAGTGGCACTTAACG 660 |
| | ************** ******** ************** ******************** |
| ZsG_AllProb | GAGCATGCTATCGCAAGCGGATCAGCACTCCCGTGA 696 |
| ZsG_APpRules | GAGCATGCTATCGCAAGCGGTTCAGCACTCCCGTGA 696 |
| | ************************************ |

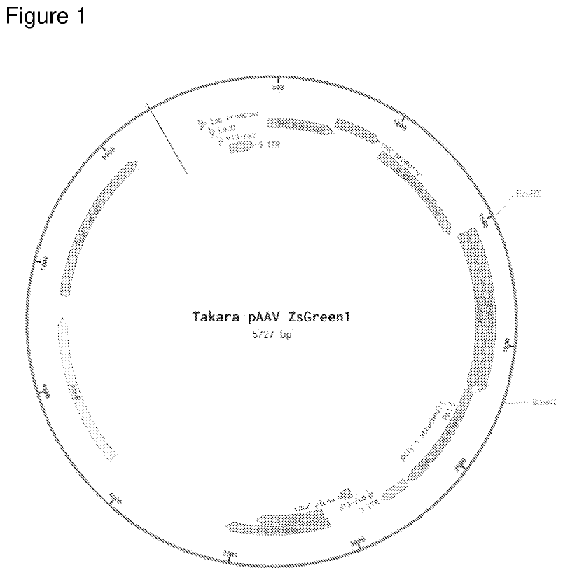

Each of the four ZsGreen fluorescent protein ORFs were then commercially ordered as synthetic double-stranded DNA (dsDNA) fragments and cloned into the plasmid pAAV ZsGreen1 between the BamHI and EcoRI restriction sites (FIG. 1). After verifying the sequences of the cloned plasmids by Sanger sequencing, the ZsGreen1 plasmid variants, along with a control pUC19 plasmid to equalize total DNA amounts, were transiently transfected into HEK293T cells in 24-well plates with lipofectamine 3000, per the manufacturer's instructions. The DNA transfection conditions are shown in Table 4 below.

TABLE 4
Transfection conditions

| Transfection | pUC19 DNA per | ZsGreen variant DNA |
|---|---|---|
| dilution condition | well (μg) | per well (μg) |
| 1:4 | 0.4 | 0.1 |
| 1:16 | 0.47 | 0.029 |

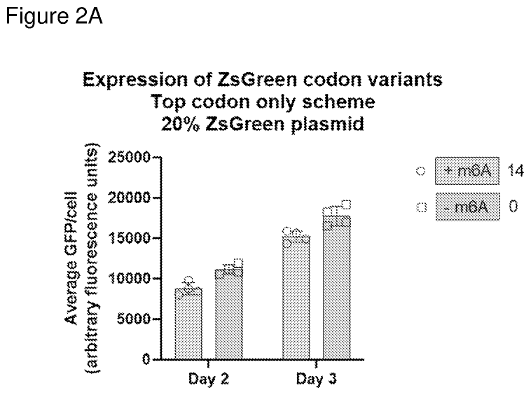

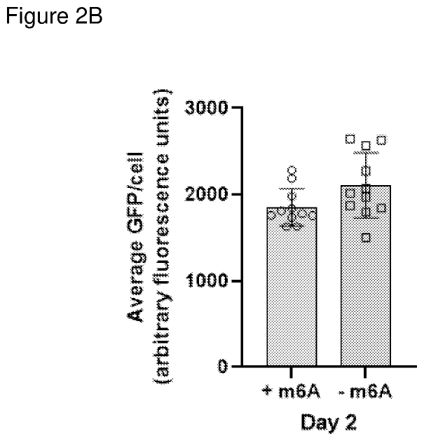

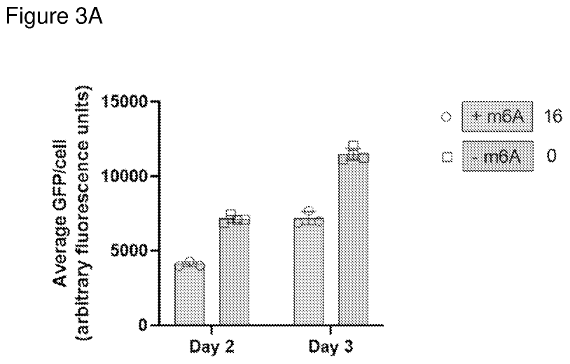

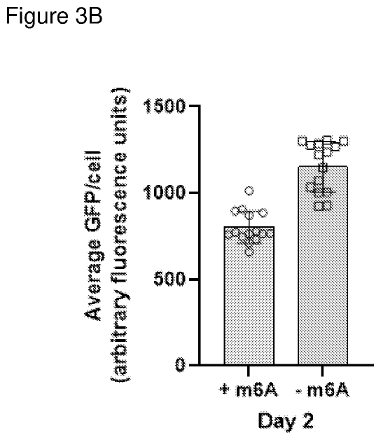

The transfected HEK293 cells were collected by trypsinization 2 or 3 days post-transfection and relative protein production by each ORF variant in both transfection conditions was analyzed by flow cytometry, in which the relative ZsGreen fluorescence in the green channel was measured. The ORFs generated from the all-best codon usage scheme with or without implementation of the six additional codon usage rules were compared following transfection (FIG. 2). The ORFs generated from the proportional usage scheme with or without implementation of the six additional codon usage rules were also compared (FIG. 3).

### Example 2. Determining GLP-1-Fc Protein Yield Using a Commercially Available Codon Optimization Webtool with or without Reducing DRACH Motifs

This example describes the process of codon optimizing a sequence encoding a GLP-1-Fc protein following codon optimization with a commercially available webtool and comparatively analyzing the resulting sequences and protein production thereof with or without applying the series of codon usage rules to reduce or eliminate DRACH motifs. The codon usage rules, or pRules, are as follows:
1. Never use GAC for aspartic acid (D)
2. Never use GAA for glutamic acid (E)
3. Never use GGA for glycine (G)
4. Never use AAA for lysine (K)
5. Never use AAC for asparagine (N)
6. Always use ACT for threonine (T)

In this example, the six codon usage rules were applied only for observed DRACH motifs and not at every instance in which D, E, G, K, N, and T codons appear in the sequence. To generate a codon optimized GLP1-Fc ORF for protein production, the amino acid sequence was entered into the IDT Codon Optimization Webtool available on the Integrated DNA Technologies website. Following entering of the amino acid sequence, the ORF of GLP-1-Fc was generated, and the DRACH motifs were quantified. The ORF of GLP-1-Fc generated by the IDT Codon Optimization Webtool (SEQ ID NO: 5) is shown below:

>IDT
ATGATCCCCGCAAAGGACATGGCGAAGGTAATGATTGTGATGCTG
GCAATCTGCTTTCTGACTAAGTCAGACGGGAAGTCAGTTAAGAAG
CGCCACGGCGAAGGCACCTTTACCAGTGATGTTAGCTCGTATCTG
GAGGAACAGGCCGCCAAAGAGTTCATCGCCTGGCTCGTGAAGGGG
GGAGGCGGAGGAGGTGGATCTGGGGGCGGTGGGTCCGGTGGTGGC
GGCTCCGCGGAATCGAAATACGGCCCTCCTTGTCCACCATGCCCC
GCTCCTGAAGCCGCAGGAGGACCCTCTGTCTTTCTCTTCCCACCT
AAGCCTAAAGATACTCTTATGATAAGTCGAACCCCTGAGGTGACA
TGCGTGGTGGTCGATGTTTCACAGGAAGACCCTGAGGTCCAGTTC
AACTGGTATGTTGATGGGGTAGAGGTACACAATGCCAAAACAAAG
CCTAGAGAGGAACAGTTCAATAGCACATATCGCGTGGTTTCCGTG
CTGACAGTGCTGCACCAGGACTGGCTCAACGGAAAGGAGTACAAG
TGTAAGGTTAGCAACAAAGGCCTTCCAAGTTCGATAGAGAAGACA
ATCTCTAAAGCTAAGGGACAGCCTAGAGAGCCACAGGTTTACACT
CTGCCCCCCAGTCAGGAGGAAATGACCAAAAACCAGGTCAGCCTG
ACTTGTCTGGTGAAGGGATTCTATCCATCAGATATTGCCGTCGAG
TGGGAAAGCAATGGGCAGCCTGAGAATAATTACAAAACCACACCC
CCCGTGCTGGATAGCGACGGCAGCTTTTTCCTATATTCCCGACTT
ACCGTTGACAAGTCCCGCTGGCAGGAAGGCAATGTGTTTTCTTGC
AGTGTGATGCACGAGGCATTGCATAATCACTATACCCAGAAAAGC
CTGAGCCTGTCCCTGGGGTGA

A total of 19 DRACH motifs appeared in the GLP-1-Fc ORF. Next, the ORF was further modified according to the six codon usage rules to modify the DRACH motifs. By following these rules, 18 DRACH motifs were removed by synonymous codon substitution. Due to cloning limitations, one DRACH motif in the sequence could not be altered and was left in the ORF with no substitutions. The resulting modified sequence in which all but one DRACH motif were removed from the IDT sequence (IDT−m6A) (SEQ ID NO: 6) is shown below:

>IDT-m6A
ATGATCCCCGCAAAGGATATGGCGAAGGTAATGATTGTGATGCTG
GCAATCTGCTTTCTGACGAAGTCAGACGGGAAGTCAGTTAAGAAG
CGCCACGGCGAAGGCACCTTTACCAGTGATGTTAGCTCGTATCTG
GAGGAGCAGGCCGCCAAAGAGTTCATCGCCTGGCTCGTGAAGGGG
GGAGGCGGAGGAGGTGGATCTGGGGGCGGTGGGTCCGGTGGTGGC
GGCTCCGCGGAATCGAAATACGGCCCTCCTTGTCCACCATGCCCC
GCTCCTGAAGCCGCAGGAGGCCCCTCTGTCTTTCTCTTCCCACCT
AAGCCTAAAGATACTCTTATGATAAGTCGAACGCCTGAGGTGACG
TGCGTGGTGGTCGATGTTTCACAGGAGGATCCTGAGGTCCAGTTC
AACTGGTATGTTGATGGGGTAGAGGTACACAATGCCAAGACGAAG
CCTAGAGAGGAGCAGTTCAATAGCACATATCGCGTGGTTTCCGTG
CTGACGGTGCTGCACCAGGATTGGCTCAACGGAAAGGAGTACAAG
TGTAAGGTTAGCAACAAAGGCCTTCCAAGTTCGATAGAGAAGACG
ATCTCTAAAGCTAAGGGCCAGCCTAGAGAGCCACAGGTTTACACT
CTGCCCCCCAGTCAGGAGGAAATGACGAAGAATCAGGTCAGCCTG
ACGTGTCTGGTGAAGGGATTCTATCCATCAGATATTGCCGTCGAG
TGGGAAAGCAATGGGCAGCCTGAGAATAATTACAAGACGACACCC
CCCGTGCTGGATAGCGACGGCAGCTTTTTCCTATATTCCCGACTT
ACCGTTGATAAGTCCCGCTGGCAGGAAGGCAATGTGTTTTCTTGC
AGTGTGATGCACGAGGCATTGCATAATCACTATACCCAGAAAAGC
CTGAGCCTGTCCCTGGGGTGA

The generated GLP1-Fc ORFs in which the DRACH motifs were left alone (IDT) (SEQ ID NO: 5) or removed following application of the six codon usage rules (IDT−m6A) (SEQ ID NO: 6) were compared and are shown as follows, in which identical nucleotides are denoted with an asterisk (*), and differences in the polynucleotide sequence are shown in bold and underline.

| | | |
|---|---|---|
| IDT | TTTGGCAAAGAATTGGGATTCGCGAGAATTCGCCACCATGATCCCCGCAAAGGACATGGC | 60 |
| IDT-m6A | TTTGGCAAAGAATTGGGATTCGCGAGAATTCGCCACCATGATCCCCGCAAAGGATATGGC | 60 |
| | ****************************************************** ***** | |
| IDT | GAAGGTAATGATTGTGATGCTGGCAATCTGCTTTCTGACTAAGTCAGACGGGAAGTCAGT | 120 |
| IDT-m6A | GAAGGTAATGATTGTGATGCTGGCAATCTGCTTTCTGACGAAGTCAGACGGGAAGTCAGT | 120 |
| | *************************************** ******************** | |
| IDT | TAAGAAGCGCCACGGCGAAGGCACCTTTACCAGTGATGTTAGCTCGTATCTGGAGGAACA | 180 |
| IDT-m6A | TAAGAAGCGCCACGGCGAAGGCACCTTTACCAGTGATGTTAGCTCGTATCTGGAGGAGCA | 180 |
| | ************************************************************ | |
| IDT | GGCCGCCAAAGAGTTCATCGCCTGGCTCGTGAAGGGGGGAGGCGGAGGAGGTGGATCTGG | 240 |
| IDT-m6A | GGCCGCCAAAGAGTTCATCGCCTGGCTCGTGAAGGGGGGAGGCGGAGGAGGTGGATCTGG | 240 |
| | ************************************************************ | |
| IDT | GGGCGGTGGGTCCGGTGGTGGCGGCTCCGCGGAATCGAAATACGGCCCTCCTTGTCCACC | 300 |
| IDT-m6A | GGGCGGTGGGTCCGGTGGTGGCGGCTCCGCGGAATCGAAATACGGCCCTCCTTGTCCACC | 300 |
| | ************************************************************ | |
| IDT | ATGCCCCGCTCCTGAAGCCGCAGGAGGACCCTCTGTCTTTCTCTTCCCACCTAAGCCTAA | 360 |
| IDT-m6A | ATGCCCCGCTCCTGAAGCCGCAGGAGGCCCCTCTGTCTTTCTCTTCCCACCTAAGCCTAA | 360 |
| | *************************** ******************************** | |
| IDT | AGATACTCTTATGATAAGTCGAACCCCTGAGGTGACATGCGTGGTGGTCGATGTTTCACA | 420 |
| IDT-m6A | AGATACTCTTATGATAAGTCGAACGCCTGAGGTGACGTGCGTGGTGGTCGATGTTTCACA | 420 |
| | ************************************ *********************** | |
| IDT | GGAAGACCCTGAGGTCCAGTTCAACTGGTATGTTGATGGGGTAGAGGTACACAATGCCAA | 480 |
| IDT-m6A | GGAGGATCCTGAGGTCCAGTTCAACTGGTATGTTGATGGGGTAGAGGTACACAATGCCAA | 480 |
| | *** ** ***************************************************** | |
| IDT | AACAAAGCCTAGAGAGGAACAGTTCAATAGCACATATCGCGTGGTTTCCGTGCTGACAGT | 540 |
| IDT-m6A | GACGAAGCCTAGAGAGGAGCAGTTCAATAGCACATATCGCGTGGTTTCCGTGCTGACGGT | 540 |
| | ** ************** ************************************** ** | |
| IDT | GCTGCACCAGGACTGGCTCAACGGAAAGGAGTACAAGTGTAAGGTTAGCAACAAAGGCCT | 600 |
| IDT-m6A | GCTGCACCAGGATTGGCTCAACGGAAAGGAGTACAAGTGTAAGGTTAGCAACAAAGGCCT | 600 |
| | ************************************************************ | |
| IDT | TCCAAGTTCGATAGAGAAGACAATCTCTAAAGCTAAGGGACAGCCTAGAGAGCCACAGGT | 660 |
| IDT-m6A | TCCAAGTTCGATAGAGAAGACGATCTCTAAAGCTAAGGGCCAGCCTAGAGAGCCACAGGT | 660 |
| | ********************* ***************** ******************** | |
| IDT | TTACACTCTGCCCCCCAGTCAGGAGGAAATGACCAAAAACCAGGTCAGCCTGACTTGTCT | 720 |
| IDT-m6A | TTACACTCTGCCCCCCAGTCAGGAGGAAATGACGAAGAATCAGGTCAGCCTGACGTGTCT | 720 |
| | ********************************* ** ** ************** ***** | |
| IDT | GGTGAAGGGATTCTATCCATCAGATATTGCCGTCGAGTGGGAAAGCAATGGGCAGCCTGA | 780 |
| IDT-m6A | GGTGAAGGGATTCTATCCATCAGATATTGCCGTCGAGTGGGAAAGCAATGGGCAGCCTGA | 780 |
| | ************************************************************ | |
| IDT | GAATAATTACAAAACCACACCCCCCGTGCTGGATAGCGACGGCAGCTTTTTCCTATATTC | 840 |
| IDT-m6A | GAATAATTACAAGACGACACCCCCCGTGCTGGATAGCGACGGCAGCTTTTTCCTATATTC | 840 |
| | ************ ** ******************************************** | |
| IDT | CCGACTTACCGTTGACAAGTCCCGCTGGCAGGAAGGCAATGTGTTTTCTTGCAGTGTGAT | 900 |
| IDT-m6A | CCGACTTACCGTTGATAAGTCCCGCTGGCAGGAAGGCAATGTGTTTTCTTGCAGTGTGAT | 900 |
| | *************** ******************************************** | |
| IDT | GCACGAGGCATTGCATAATCACTATACCCAGAAAAGCCTGAGCCTGTCCCTGGGGTGATA | 960 |
| IDT-m6A | GCACGAGGCATTGCATAATCACTATACCCAGAAAAGCCTGAGCCTGTCCCTGGGGTGATA | 960 |
| | ************************************************************ | |
| IDT | AGGATCCACGGGTGGCATCCCTGTGACCCCTC | 992 |
| IDT-m6A | AGGATCCACGGGTGGCATCCCTGTGACCCCTC | 992 |
| | ******************************** | |

The two ORFs shown above were ordered as synthetic dsDNA fragments and cloned into the pAAV ZsGreen1 plasmid between the BamHI and EcoRI restriction sites (FIG. 1). After verifying the sequences of the cloned plasmids by Sanger sequencing, the plasmids containing the ORF variants, along with a control pUC19 plasmid to for a total amount of 0.5 μg of DNA, were transiently transfected into HEK293T cells in 24-well plates with lipofectamine 3000, per the manufacturer's instructions. The DNA transfection conditions are shown in Table 5 below.

TABLE 5
Transfection Conditions

| Transfection dilution | pUC 19 DNA per | GLP-1-Fc ORF variant DNA |
|---|---|---|
| condition | well (μg) | per well (μg) |
| 1:10 | 0.45 | 0.05 |

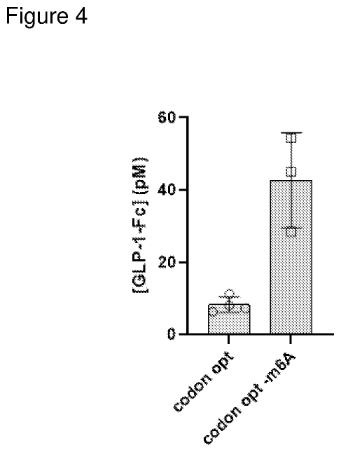

Following 2 days post-transfection, the cell culture supernatants were collected, and the amount of secreted GLP-1-Fc protein was quantified via ELISA. The concentration of detected GLP-1-Fc protein increased by more than 5-fold in the sample in which DRACH motifs were reduced, as compared to the sample that was codon optimized only using the IDT webtool (FIG. 4).

### Example 3. Determining GLP-1-Fc Protein Production Yield Via AAV Transduction Following Elimination of DRACH Motifs

This example describes the process of codon optimizing a sequence encoding a GLP-1-Fc protein following codon optimization in which all amino acids in the ORF were encoded by the most preferred codon (i.e., the highest usage frequency) in every instance. The codon optimized ORF was then subjected to the codon usage rules to eliminate DRACH motifs. The codon usage rules, or pRules, are as follows:
1. Never use GAC for aspartic acid (D)
2. Never use GAA for glutamic acid (E)
3. Never use GGA for glycine (G)
4. Never use AAA for lysine (K)
5. Never use AAC for asparagine (N)
6. Always use ACT for threonine (T)
   The codon optimized ORF encoding GLP-1-Fc (denoted CH) (SEQ ID NO: 7) contains 19 DRACH motifs and is shown below:

>CH
atgatccccgccaaggacatggccaaggtgatgatcgtgatgctgg
ccatctgcttcctgaccaagtccgacggcaagtccgtgaagaagc
gccacggcgagggcaccttcaccagcgacgtgtctagctacctgg
aggagcaggccgccaaggagttcatcgcctggctggtgaagggcg
gcggcggcggcggcggcagcggcggcggcggcagcggcggcggcg
gcagcgccgagagcaagtacggccccccctgccccccctgccccg
cccccgaggccgccggeggccccagcgtgttcctgttccccccca
agcccaaggacaccctgatgatcagcaggacccccgaggtgacct
gcgtggtggtggacgtgagccaggaggaccccgaggtgcagttca
actggtacgtggacggcgtggaggtgcacaacgccaagaccaagc
ccagggaggagcagttcaacagcacctacagggtggtgagcgtgc
tgaccgtgctgcaccaggactggctgaacggcaaggagtacaagt
gcaaggtttctaacaagggcctgcccagcagcatcgagaagacca
tcagcaaggccaagggccagcccagggagccccaggtgtacaccc
tgccccccagccaggaggagatgaccaagaaccaagttagcctga
cctgcctggtgaagggcttctaccccagcgacatcgccgtggagt
gggagagcaacggccagcccgagaacaactacaagaccacccccc
ccgtgctggacagcgacggcagcttcttcctgtacagccgactga
ccgtggacaagagcaggtggcaggagggcaacgtgttcagctgca
gcgtgatgcacgaggccctgcacaaccactacacccagaagagcc
tgagcctgagcctgggctga

The CH ORF was further modified by following the six codon usage rules to eliminate DRACH motifs, which resulted in 20 base pair substitutions and the removal of all 19 DRACH motifs that were present in the codon optimized CH ORF. The further modified ORF encoding GLP-1-Fc in which all DRACH motifs were removed (denoted CH−m6A) (SEQ ID NO: 8) is shown below with all modified base pairs shown in bold and underline:

>CH-m6A
atg atc ccc gcc aag gaT atg gcc aag gtg atg atc gtg atg ctg gcc atc tgc ttc
CTG ACG aag tcc gac ggc aag tcc gtg aag aag cgc cac ggc gag ggc acc ttc acc
agc gac gtg tct agc tac ctg gag gag cag gcc gcc aag gag ttc atc gcc tgg ctg
gtg aag ggc ggc ggc ggc ggc ggc ggc agc ggc ggc ggc ggc agc ggc ggc ggc ggc
agc gcc gag agc aag tac ggc ccc ccc tgc ccc ccc tgc ccc gcc ccc gag gcc gcc
ggc ggc ccc agc gtg ttc ctg ttc ccc ccc aag ccc aag gaT acc ctg atg atc agc
aGG ACG ccc gag gTG ACG tgc gtg gtg gtg gac gtg agc cag gaG GAT Ccc gag gtg
cag ttc aac tgg tac gtg gac ggc gtg gag gtg cac aac gcc aAG ACG aag ccc agg
gag gag cag ttc aac agc acc tac agg gtg gtg agc gtg cTG ACG gtg ctg cac cag
gaT tgg ctg aac ggc aag gag tac aag tgc aag gtt tcT AAT Aag ggc ctg ccc agc
agc atc gag aAG ACG atc agc aag gcc aag ggc cag ccc agg gag ccc cag gtg tac
acc ctg ccc ccc agc cag gag gag aTG ACG aag aaT caa gtt agc CTG ACG tgc ctg
gtg aag ggc ttc tac ccc agc gac atc gcc gtg gag tgg gag agc aac ggc cag ccc
gaG AAT AaT tac aAG ACG acc ccc ccc gtg ctg gaT agc gac ggc agc ttc ttc ctg
tac agc cga CTG ACG gtg gaT aag agc agg tgg cag gag ggc aac gtg ttc agc tgc
agc gtg atg cac gag gcc ctg cac aac cac tac acc cag aag agc ctg agc ctg agc
ctg ggc tga

Alignment of the codon optimized GLP-1-Fc ORF (CH) (SEQ ID NO: 7) and the codon optimized GLP-1-Fc ORF with removal of the DRACH motifs (CH−m6A) sequences (SEQ ID NO: 8) are shown below, in which identical nucleotides are denoted with an asterisk (*), and the CH−m6A sequence is shown with the stretch of nucleotides that comprised the DRACH motif as capital letters and the modified nucleotide in bold and underline:

| | | |
|---|---|---|
| CH | atgatccccgccaaggacatggccaaggtgatgatcgtgatgctggccatctgcttcctg | 60 |
| CH-m6A | atgatccccgccaaggaTatggccaaggtgatgatcgtgatgctggccatctgcttccTG | 60 |
| | ***************** ****************************************** | |
| CH | accaagtccgacggcaagtccgtgaagaagcgccacggcgagggcaccttcaccagcgac | 120 |
| CH-m6A | ACGaagtccgacggcaagtccgtgaagaagcgccacggcgagggcaccttcaccagcgac | 120 |
| | ** ********************************************************* | |
| CH | gtgtctagctacctggaggagcaggccgccsaggagttcatcgcctggctggtgaagggc | 180 |
| CH-m6A | gtgtctagctacctggaggagcaggccgccsaggagttcatcgcctggctggtgaagggc | 180 |
| | ************************************************************ | |
| CH | ggcggcggcggcggcggcagcggcggcggcggcagcggcggcggcggcagcgccgagagc | 240 |
| CH-m6A | ggcggcggcggcggcggcagcggcggcggcggcagcggcggcggcggcagcgccgagagc | 240 |
| | ************************************************************ | |
| CH | aagtacggccccccctgccccccctgccccgcccccgaggccgccggcggccccagcgtg | 300 |
| CH-m6A | aagtacggccccccctgccccccctgccccgcccccgaggccgccggcggccccagcgtg | 300 |
| | ************************************************************ | |
| CH | ttcctgttcccccccaagcccaaggacaccctgatgatcagcaggacccccgaggtgacc | 360 |
| CH-m6A | ttcctgttcccccccaagcccaaggaTaccctgatgatcagcaGGACGcccgaggTGACG | 360 |
| | ************************** ******************** ******* **** | |
| CH | tgcgtggtggtggacgtgagccaggaggaccccgaggtgcagttcaactggtacgtggac | 420 |
| CH-m6A | tgcgtggtggtggacgtgagccaggaGGATCccgaggtgcagttcaactggtacgtggac | 420 |
| | ***************************** ****************************** | |
| CH | ggcgtggaggtgcacaacgccaagaccaagcccagggaggagcagttcaacagcacctac | 480 |
| CH-m6A | ggcgtggaggtgcacaacgccaAGACGaagcccagggaggagcagttcaacagcacctac | 480 |
| | ************************** ********************************* | |
| CH | agggtggtgagcgtgctgaccgtgctgcaccaggactggctgaacggcaaggagtacaag | 540 |
| CH-m6A | agggtggtgagcgtgcTGACGgtgctgcaccaggaTtggctgaacggcaaggagtacaag | 540 |
| | ******************** ************** ************************ | |
| CH | tgcaaggtttctaacaagggcctgcccagcagcatcgagaagaccatcagcaaggccaag | 600 |
| CH-m6A | tgcaaggtttcTAATAagggcctgcccagcagcatcgagaAGACGatcagcaaggccaag | 600 |
| | ************** ***************************** *************** | |
| CH | ggccagcccagggagccccaggtgtacaccctgccccccagccaggaggagatgaccaag | 660 |
| CH-m6A | ggccagcccagggagccccaggtgtacaccctgccccccagccaggaggagaTGACGaag | 660 |
| | ************************************************************ | |
| CH | aaccaagttagcctgacctgcctggtgaagggcttctaccccagcgacatcgccgtggag | 720 |
| CH-m6A | aaTcaagttagccTGACGtgcctggtgaagggcttctaccccagcgacatcgccgtggag | 720 |
| | ** ************** ****************************************** | |
| CH | tgggagagcaacggccagcccgagaacaactacaagaccaccccccccgtgctggacagc | 780 |
| CH-m6A | tgggagagcaacggccagcccgaGAATAaTtacaAGACGaccccccccgtgctggaTagc | 780 |
| | ************************** ** ******** ***************** *** | |
| CH | gacggcagcttcttcctgtacagccgactgaccgtggacaagagcaggtggcaggagggc | 840 |
| CH-m6A | gacggcagcttcttcctgtacagccgacTGACGgtggaTaagagcaggtggcaggagggc | 840 |
| | ******************************** ***** ********************* | |
| CH | aacgtgttcagctgcagcgtgatgcacgaggccctgcacaaccactacacccagaagagc | 900 |
| CH-m6A | aacgtgttcagctgcagcgtgatgcacgaggccctgcacaaccactacacccagaagagc | 900 |
| | ************************************************************ | |
| CH | ctgagcctgagcctgggctga | 921 |
| CH-m6A | ctgagcctgagcctgggctga | 921 |
| | ********************* | |

After validating the polynucleotide sequences of the ORF, the amino acid sequences encoded by each ORF were aligned to ensure that the GLP-1-Fc protein retained 100% sequence identity after elimination of the DRACH motifs in the ORF. The alignment of the amino acid sequences from the CH ORF (SEQ ID NO: 9) and the CH−m6a ORF (SEQ ID NO: 10) are shown below, in which identical amino acids are denoted with an asterisk (*):

| | | |
|---|---|---|
| CH | MIPAKDMAKVMIVMLAICFLIKSDGKSVKKRHGEGTFISDVSSYLEEQAAKEFIAWLVKG | 60 |
| CH-m6A | MIPAKDMAKVMIVMLAICELIKSDGKSVKKRHGEGIFTSDVSSYLEEQAAKEFIAWLVKG | 60 |
| | ************************************************************ | |
| CH | GGGGGGSGGGGSGGGGSAESKYGPPCPPCPAPEAAGGPSVFLEPPKPKDTLMISRTPEVT | 120 |
| CH-m6A | GGGGGGSGGGGSGGGGSAESKYGPPCPPCPAPEAAGGPSVFLFPPKPKDTLMISRTPEVT | 120 |
| | ************************************************************ | |
| CH | CVVVDVSQEDPEVQFNWYVDGVEVHNAKIKPREEQENSTYRVVSVLIVLHQDWLNGKEYK | 180 |
| CH-m6A | CVVVDVSQEDPEVQFNWYVDGVEVHNAKIKPREEQFNSTYRVVSVLIVLHQDWLNGKEYK | 180 |
| | ************************************************************ | |
| CH | CKVSNKGLPSSIEKTISKAKGQPREPQVYILPPSQEEMTKNQVSLICLVKGFYPSDIAVE | 240 |
| CH-m6A | CKVSNKGLPSSIEKTISKAKGQPREPQVYILPPSQEEMTKNQVSLICLVKGFYPSDIAVE | 240 |
| | ************************************************************ | |
| CH | WESNGQPENNYKTTPPVLDSDGSFFLYSRLTVDKSRWQEGNVESCSVMHEALANHYTQKS | 300 |
| CH-m6A | WESNGQPENNYKTTPPVLDSDGSFFLYSRLTVDKSRWQEGNVESCSVMHEALHNHYTQKS | 300 |
| | ************************************************************ | |
| CH | LSLSLG* | 306 |
| CH-m6A | LSLSLG* | 306 |
| | ******* | |

After confirming the polynucleotide and amino acid sequences, both the CH and CH−m6A GLP-1-Fc ORFS (SEQ ID NOs: 7 and 8) were ordered as synthetic dsDNA fragments and were subsequently cloned into the pAAV ZsGreen1 plasmid between the BamHI and EcoRI restriction sites. Sequences of the cloned plasmids were verified by Sanger sequencing.

The CH and CH−m6A plasmids as well as the IDT and IDT−m6A plasmids described in Example 2 were then to construct AAV9 vectors by triple transfection into HEK293 cells using standard methods known in the art. The recovered AAV9.GLP-1Fc vectors were titered by qPCR to determine the vector genome concentrations and then applied to HEK293 cells at a multiplicity of infection (MOI) of 1×10⁶ vector genomes per cell for GLP-1-Fc protein transduction. After 3 days of protein transduction, the supernatants of the HEK293 cell cultures were collected, and the levels of secreted GLP-1-Fc protein were determined by ELISA.

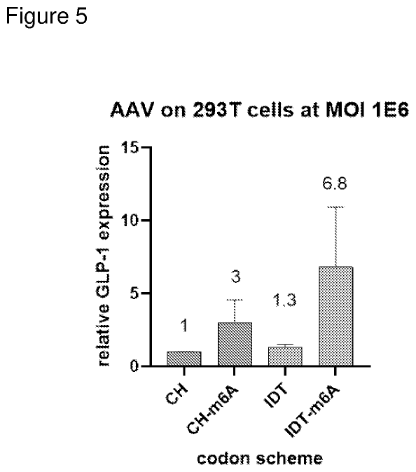

The GLP-1-Fc protein levels in each sample were compared relative to the CH codon optimized ORF sample (FIG. 5). The CH−m6A sample in which all DRACH motifs were removed from the codon optimized CH ORF (SEQ ID NO: 8) resulted in a 3-fold increase in detectable GLP-1-Fc protein secretion relative to the CH sample, in which the CH ORF (SEQ ID NO: 7) was used. The codon optimized ORF using the IDT codon optimization webtool (SEQ ID NO: 5) exhibited a 1.3-fold increase in detectable GLP-1-Fc protein secretion relative to the CH sample. Additionally, the IDT codon optimized ORF in which all but one DRACH motif was removed (SEQ ID NO: 6) resulted in a 6.8-fold increase in detectable GLP-1-Fc protein secretion relative to the CH sample.

### Example 4. Evaluating Circulating GLP-1-Fc Protein In Vivo Following AAV Delivery

This example describes the impact that the different codon optimization methodologies described in Examples 2 and 3 have on in vivo protein production.

In addition to the 4 AAV vectors prepared as described in Example 3, a native ORF encoding GLP-1-Fc (i.e., an ORF without the application of any codon optimization method) was generated based on the human GLP-1 gene sequence. The native GLP-1-Fc ORF (SEQ ID NO: 11) is shown below:

>native
ATGATACCTGCAAAAGACATGGCTAAAGTTATGATTGTCATGTTGG
CAATTTGTTTTCTTACAAAATCGGATGGGAAATCTGTAAAGAAGC
GGCATGGAGAAGGGACCTTTACCAGTGATGTAAGTTCTTATTTGG
AAGAACAAGCTGCCAAGGAATTCATTGCTTGGCTGGTGAAAGGCG
GAGGAGGGGGGGGGGGGTCAGGCGGAGGGGGCTCCGGAGGAGGAG
GATCTGCAGAGTCCAAATATGGTCCCCCATGCCCACCATGCCCAG
CACCTGAGGCTGCAGGGGGACCATCAGTCTTCCTGTTCCCCCCAA
AACCCAAGGACACTCTCATGATCTCCCGGACCCCTGAGGTCACGT
GCGTGGTGGTGGACGTGAGCCAGGAAGACCCCGAGGTCCAGTTCA
ACTGGTACGTGGATGGCGTGGAGGTGCATAATGCCAAGACAAAGC
CGCGGGAGGAGCAGTTCAACAGCACGTACCGTGTGGTCAGCGTCC
TCACCGTCCTGCACCAGGACTGGCTGAACGGCAAGGAGTACAAGT
GCAAGGTCTCCAACAAAGGCCTCCCGTCCTCCATCGAGAAAACCA
TCTCCAAAGCCAAAGGGCAGCCCCGAGAGCCACAGGTGTACACCC
TGCCCCCATCCCAGGAGGAGATGACCAAGAACCAGGTCAGCCTGA
CCTGCCTGGTCAAAGGCTTCTACCCCAGCGACATCGCCGTGGAGT
GGGAGAGCAATGGGCAGCCGGAGAACAACTACAAGACCACGCCTC
CCGTGCTGGACTCCGACGGCTCCTTCTTCCTCTACAGCAGGCTCA
CCGTGGACAAGAGCAGGTGGCAGGAGGGGAATGTCTTCTCATGCT
CCGTGATGCATGAGGCTCTGCACAACCACTACACACAGAAGAGCC
TCTCCCTGTCTCTGGGTTGA

The plasmid and AAV vector encoding the native GLP-1-Fc sequence were generated and validated using the same methods as described in Example 3.

To test in vivo protein production of the four codon optimization methods described, each of the described GLP-1-Fc AAV vectors were injected into a mouse at either a low-dose (1×10⁹ genome copies (GC) per mouse) or a high-dose (1×10¹⁰ GC per mouse). Eight 8-week-old female rag mice comprised each vector and dosing experimental group.

The mice were each injected with a single 10 μL intramuscular injection of a GLP-1-Fc AAV vector in the leg. A serum sample was collected from the mice every 2 weeks for 60 days following the injection. The muscle tissue at the injection site as well as a liver sample were harvested at necropsy, as these tissues were the primary organs for protein transduction following intramuscular AAV administration. Serum protein levels of GLP-1-Fc were evaluated by ELISA, and GLP-1-Fc mRNA transcript levels in muscle and liver tissue homogenates were measured by qPCR.

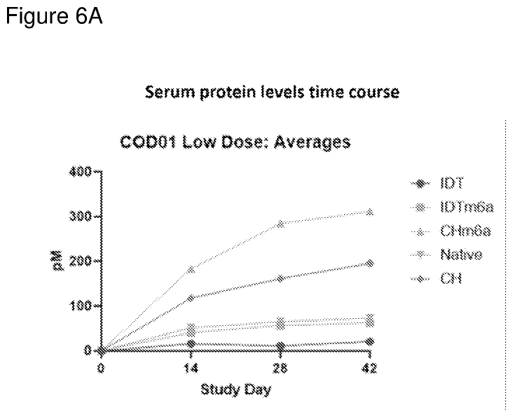

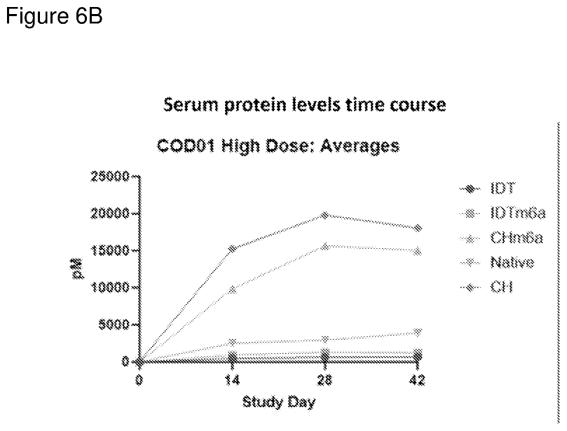

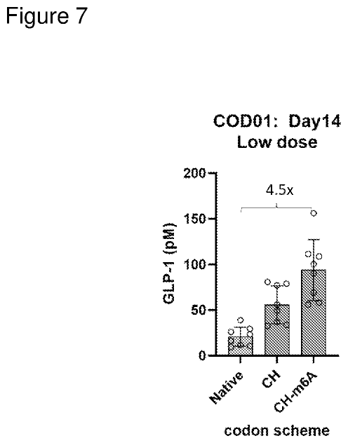

In both dosing groups, the CH−m6A and CH codon optimized AAV vectors produced the highest concentrations of detectable GLP-1-Fc protein in the serum at every time point over 42 days (FIGS. 6A-6B). At day 14, GLP-1-Fc protein levels in the serum were the lowest in the samples containing the vector encoding the native sequence and were the highest in samples containing the vector encoding the CH codon optimized sequence in which DRACH motifs were removed (CH−m6A) (SEQ ID NO: 8). The vector encoding the CH−m6A sequence (SEQ ID NO: 8) led to serum concentrations of GLP-1-Fc protein that were 4.5-fold higher as compared to the vector encoding the native sequence (FIG. 7).

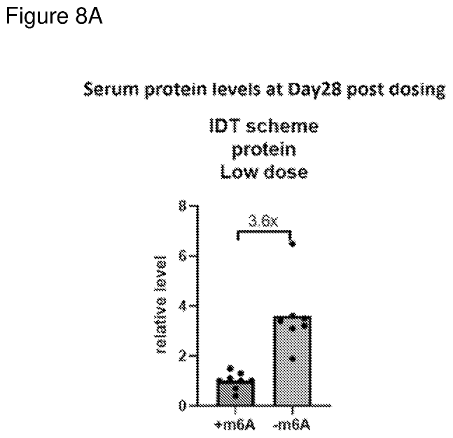

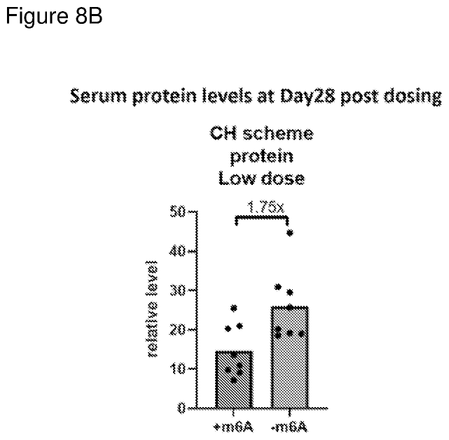

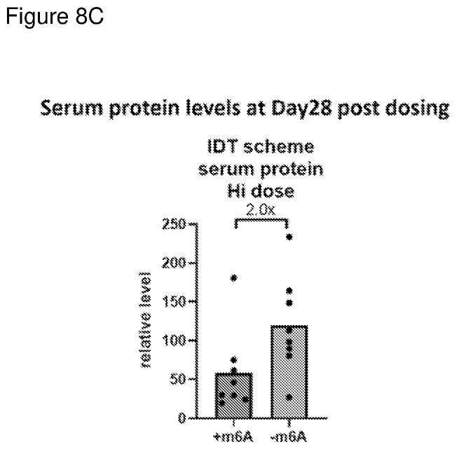

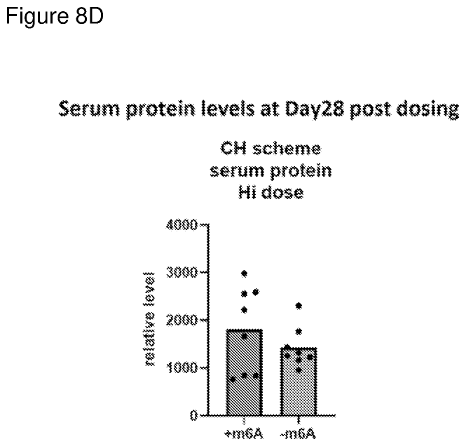

To further evaluate the impact of m6A modifications (i.e., the presence of DRACH motifs) on protein production efficiency, detectable levels of GLP-1-Fc protein in the serum were pairwise compared based on the codon optimization strategy and dosing group at 28 days following AAV vector administration (FIGS. 8A-D). Three out of the four of the compared experimental groups showed increased GLP-1-Fc protein levels in the serum when DRACH motifs were removed. The IDT codon optimization scheme following the removal of DRACH motifs (SEQ ID NO: 6) resulted in a 3.6-fold increase in serum GLP-1-Fc protein levels compared to the IDT codon optimization scheme alone (SEQ ID NO: 5) following low-dose injection of each AAV vector (FIG. 8A). The CH codon optimization scheme following the removal of DRACH motifs (SEQ ID NO: 8) resulted in a 1.75-fold increase in serum GLP-1-Fc protein levels compared to the CH codon optimization scheme alone (SEQ ID NO: 7) following the low-dose injection of each AAV vector (FIG. 8B). Similarly, the at high-doses, the IDT codon optimization scheme following the removal of DRACH motifs (SEQ ID NO: 6) resulted in a 2.0-fold increase in GLP-1-Fc protein levels compared to the ID codon optimization scheme alone (SEQ ID NO: 5) (FIG. 8D). However, no significant differences in GLP-1-Fc protein levels in the serum were observed with the high-dose injection of either variation of the CH codon optimized vectors.

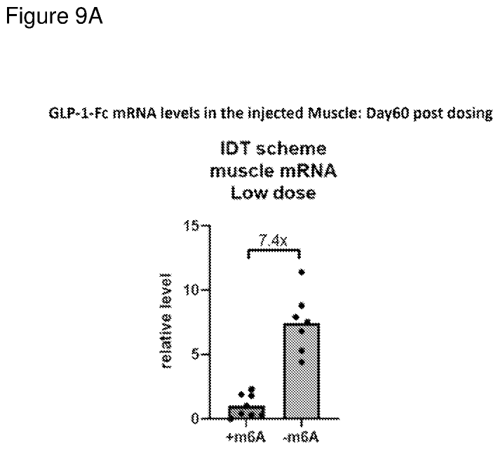

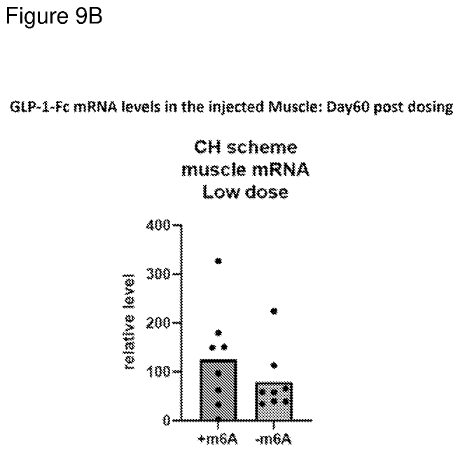

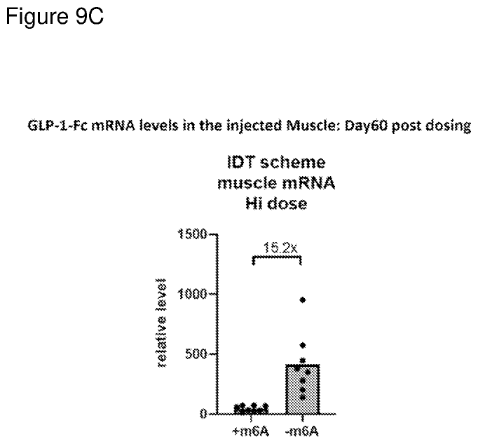

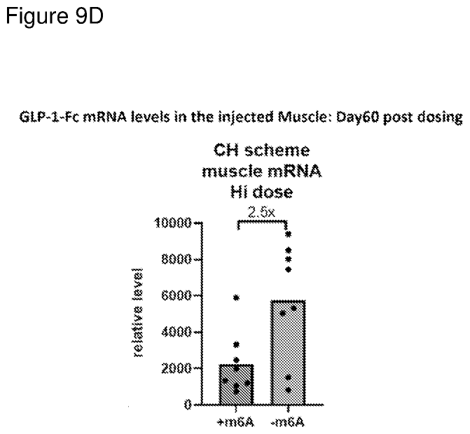

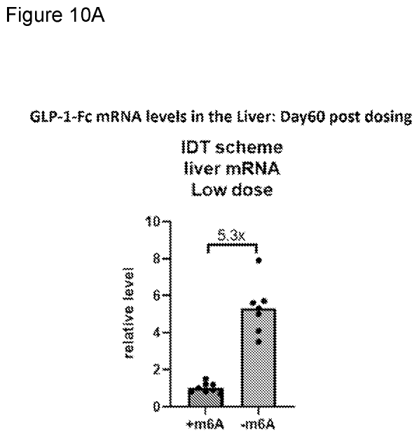

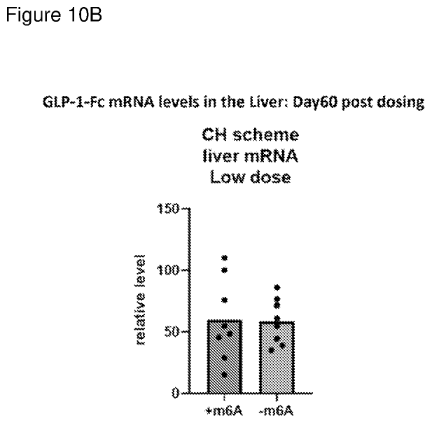

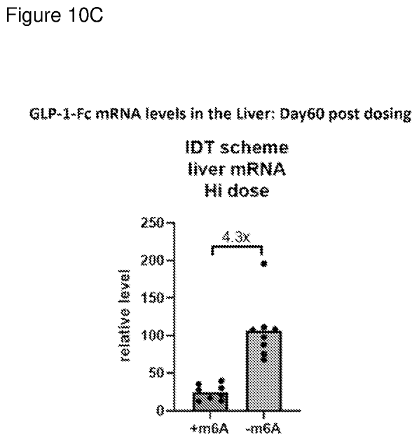

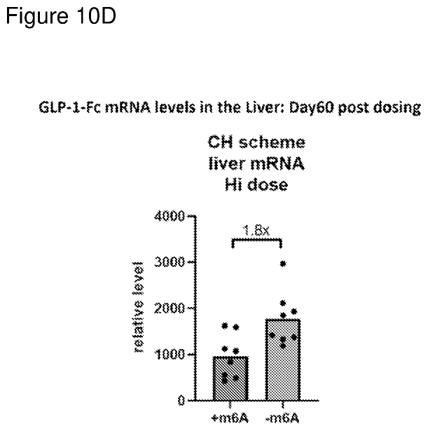

A similar trend was observed for the GLP-1-Fc mRNA transcript levels in both the muscle and liver tissue. In every group but the low-dose CH codon optimized vector, vectors in which DRACH motifs were removed following codon optimization yielded higher detectable GLP-1-Fc mRNA transcripts (FIGS. 9-10). The largest difference in tissue GLP-1-Fc mRNA transcript expression was observed in the muscle tissue following the high-dose injection of the IDT codon optimized vectors, in which removing the DRACH motifs from the codon optimized ORF led to a 15.2-fold increase in detectable GLP-1-Fc mRNA levels after 60 days (FIG. 9C). Collectively, these results indicate that the removal of DRACH motifs can lead to an increase in the expression of both mRNA transcripts and protein following transduction.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of codon optimization of a polynucleotide sequence that encodes a polypeptide of interest, the method comprising substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide.

2. The method of claim 1, wherein the polynucleotide sequence comprises a plurality of DRACH motifs, and wherein the substituting is performed only in a subset of the DRACH motifs.

3. The method of claim 2, wherein the substituting is performed only in a DRACH motif that is separated from a splice junction site within the polynucleotide sequence by at least 10 nucleotides.

4. The method of claim 3, wherein the substituting is performed only in a DRACH motif that is separated from a splice junction site within the polynucleotide sequence by at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 110 nucleotides, at least 120 nucleotides, at least 130 nucleotides, at least 140 nucleotides, at least 150 nucleotides, at least 160 nucleotides, at least 170 nucleotides, at least 180 nucleotides, at least 190 nucleotides, at least 200 nucleotides, or more.

5. The method of claim 2, wherein the substituting is performed only in a DRACH motif that is separated from a splice junction site within the polynucleotide sequence by from about 10 nucleotides to about 200 nucleotides, by from about 20 nucleotides to about 190 nucleotides, by from about 30 nucleotides to about 180 nucleotides, by from about 40 nucleotides to about 170 nucleotides, by from about 50 nucleotides to about 160 nucleotides, by from about 60 nucleotides to about 150 nucleotides, by from about 70 nucleotides to about 140 nucleotides, by from about 80 nucleotides to about 130 nucleotides, by from about 90 nucleotides to about 120 nucleotides, or by about 100 nucleotides.

6. The method of any one of claims 1-5, wherein the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC.

7. The method of claim 6, wherein the substituting is performed in a DRACH motif selected from the group consisting of AGACA, AGACT, GAACC, GAACT, GGACA, GGACT, and TGACT.

8. The method of any one of claims 1-7, wherein the substituting of the one or more nucleotides in the DRACH motif is performed using one or more codon usage guidelines selected from the group consisting of:
(a) never using the codon GAC to encode aspartic acid when eliminating the DRACH motif;
(b) never using the codon GAA to encode glutamic acid when eliminating the DRACH motif;
(c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif;
and/or
(f) always using the codon ACG to encode threonine when eliminating the DRACH motif.

9. The method of any one of claims 1-8, wherein the codon optimization further comprises implementing, across the full polynucleotide sequence, one or more codon usage guidelines selected from the group consisting of:
(a) never using the codon GAC to encode aspartic acid across the full polynucleotide sequence;
(b) never using the codon GAA to encode glutamic acid across the full polynucleotide sequence;
(c) never using the codon GGA to encode glycine across the full polynucleotide sequence;
(d) never using the codon AAA to encode lysine across the full polynucleotide sequence;
(e) never using the codon AAC to encode asparagine across the full polynucleotide sequence; and/or
(f) always using the codon ACG to encode threonine across the full polynucleotide sequence.

10. The method of any one of claims 1-9, wherein prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

11. The method of any one of claims 1-10, wherein prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism.

12. The method of claim 10 or 11, wherein the target organism is a mammal, optionally wherein the mammal is a human.

13. The method of any one of claims 1-12, wherein prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

14. The method of any one of claims 1-13, wherein prior to the substituting of the one or more nucleotides in the DRACH motif, the polynucleotide sequence has been codon optimized by modulating polynucleotide guanine-cytosine (GC) content, polynucleotide secondary structure, mRNA motifs, ribosomal binding sites, and/or rates of translation.

15. The method of claim 14, wherein the polynucleotide GC content is adjusted to between 50% and 80%.

16. The method of claim 15, wherein the polynucleotide GC content is adjusted to between 65% and 75%.

17. The method of any one of claims 1-16, wherein after the substituting of the one or more nucleotides in the DRACH motif, the method of codon optimization further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

18. The method of any one of claims 1-16, wherein after the substituting of the one or more nucleotides in the DRACH motif, the method of codon optimization further comprises substituting one or more codons in the polynucleotide sequence with an equivalent quantity of synonymous codons that occur naturally at a higher frequency in a target organism relative to the frequency with which the one or more unmodified codons occur in the target organism.

19. The method of claim 17 or 18, wherein the target organism is a mammal, optionally wherein the mammal is a human.

20. The method of any one of claims 1-19, wherein after the substituting of the one or more nucleotides in the DRACH motif, the method of codon optimization further comprises codon optimizing by way of a codon optimization webtool, codon optimization software, quantum computing, a heuristic scoring method, a neural network, or a combination thereof.

21. The method of any one of claims 1-20, wherein after the substituting of the one or more nucleotides in the DRACH motif, the method of codon optimization further comprises modulating polynucleotide GC content, polynucleotide secondary structure, mRNA motifs, ribosomal binding sites, and/or rates of translation.

22. The method of claim 21, wherein the polynucleotide GC content is adjusted to between 50% and 80%.

23. The method of claim 22, wherein the polynucleotide GC content is adjusted to between 65% and 75%.

24. The method of anyone of claims 1-23, wherein the amino acid sequence of the protein or polypeptide shares at least 85% sequence identity to a wild-type amino acid sequence of the protein or polypeptide following codon optimization.

25. The method of any one of claims 1-24, wherein the amino acid sequence of the protein or polypeptide shares 100% sequence identity to a wild-type amino acid sequence of the protein or polypeptide following codon optimization.

26. The method of any one of claims 1-25, wherein the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method.

27. The method of claim 26, wherein the number of DRACH motifs in the polynucleotide sequence is reduced by 100%, as compared to a corresponding polynucleotide sequence that has not been subjected to the method.

28. The method of any one of claims 1-27, wherein the method of codon optimization results in increased mRNA transcript stability or half-life.

29. The method of claim 28, wherein the increased mRNA transcript stability or half-life is assessed by an increase in concentration or relative abundance as compared to a corresponding polynucleotide sequence that has not been subjected to the method.

30. The method of claim 29, wherein the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

31. The method of claim 28, wherein the increased mRNA transcript stability or half-life is assessed by reduced rates of decay as detected by one or more pulse-chase methodologies.

32. The method of claim 1-31, wherein reducing the number of DRACH motifs in the polynucleotide sequence reduces the number of N(6)-methyladenosine (m6A) modifications in an mRNA transcript as compared to a corresponding polynucleotide sequence that has not been subjected to the method.

33. The method of any one of claims 1-32, wherein the method of codon optimization results in increased protein expression or stability as compared to the protein encoded by a corresponding polynucleotide sequence that has not been subjected to the method.

34. The method of any one of claims 26-33, wherein the corresponding polynucleotide sequence is a wild-type polynucleotide sequence.

35. The method of claim 33 or 34, wherein the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a corresponding polynucleotide sequence that has not been subjected to the method.

36. The method of any one of claims 33-35, wherein the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a corresponding polynucleotide sequence that has not been subjected to the method.

37. The method of any one of claims 33-36, wherein the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

38. A polynucleotide produced by the method of any one of claims 1-37.

39. A method of delivering a polypeptide-encoding polynucleotide sequence to a host cell, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the resulting polynucleotide sequence to the host cell.

40. A method of expressing an mRNA transcript in a host cell from a polypeptide-encoding polynucleotide sequence, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the polynucleotide sequence to the host cell.

41. A method of expressing a protein in a host cell from a polynucleotide sequence encoding the protein, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded protein, and (ii) providing the polynucleotide sequence to the host cell.

42. A method of delivering a polypeptide-encoding polynucleotide sequence to a subject, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the resulting polynucleotide sequence to the subject.

43. A method of expressing an mRNA transcript in a subject from a polypeptide-encoding polynucleotide sequence, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded polypeptide, and (ii) providing the polynucleotide sequence to the subject.

44. A method of expressing a protein in a subject from a polynucleotide sequence encoding the protein, the method comprising (i) substituting one or more nucleotides in a DRACH motif within the polynucleotide sequence with an equivalent quantity of alternative nucleotides that eliminate the DRACH motif, wherein the substituting does not alter the amino acid sequence of the encoded protein, and (ii) providing the polynucleotide sequence to the subject.

45. The method of any one of claims 39-44, wherein the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC.

46. The method of claim 45, wherein the substituting is performed in a DRACH motif is selected from the group consisting of AGACA, AGACT, GAACC, GAACT, GGACA, GGACT, and TGACT.

47. The method of any one of claims 39-46, wherein the substituting of the one or more nucleotides in the DRACH motif is performed using one or more codon usage guidelines selected from the group consisting of:
(a) never using the codon GAC to encode aspartic acid when eliminating the DRACH motif;
(b) never using the codon GAA to encode glutamic acid when eliminating the DRACH motif;
(c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif; and/or
(f) always using the codon ACG to encode threonine when eliminating the DRACH motif.

48. The method of any one of claims 39-47, wherein the codon optimization further comprises implementing, across the full polynucleotide sequence, one or more codon usage guidelines selected from the group consisting of:
(a) never using the codon GAC for aspartic acid across the full polynucleotide sequence;
(b) never using the codon GAA for glutamic acid across the full polynucleotide sequence;
(c) never using the codon GGA for glycine across the full polynucleotide sequence;
(d) never using the codon AAA for lysine across the full polynucleotide sequence;
(e) never using the codon AAC for asparagine across the full polynucleotide sequence; and/or
(f) always using the codon ACG for threonine across the full polynucleotide sequence.

49. The method of any one of claims 39-48, wherein prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

50. The method of any one of claims 39-49, wherein prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism.

51. The method of claim 49 or 50, wherein the target organism is a mammal, optionally wherein the mammal is a human.

52. The method of any one of claims 39-51, wherein prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

53. The method of any one of claims 39-52, wherein prior to the substituting of the one or more nucleotides in the DRACH motif, the polynucleotide sequence has been codon optimized by modulating polynucleotide GC content, polynucleotide secondary structure, mRNA motifs, ribosomal binding sites, and/or rates of translation.

54. The method of claim 53, wherein the polynucleotide GC content is adjusted to between 50% and 80%.

55. The method of claim 54, wherein the polynucleotide GC content is adjusted to between 65% and 75%.

56. The method of any one of claims 39-55, wherein after the substituting of the one or more nucleotides in the DRACH motif, the method further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

57. The method of any one of claims 39-56, wherein after the substituting of the one or more nucleotides in the DRACH motif, the method further comprises substituting one or more codons in the polynucleotide sequence with an equivalent quantity of synonymous codons that occur naturally at a higher frequency in a target organism relative to the frequency with which the one or more unmodified codons occur in the target organism.

58. The method of claim 56 or 57, wherein the target organism is a mammal, optionally wherein the mammal is a human.

59. The method of any one of claims 39-58, wherein after the substituting of the one or more nucleotides in the DRACH motif, the method further comprises codon optimizing by way of a codon optimization webtool, codon optimization software, quantum computing, a heuristic scoring method, a neural network, or a combination thereof.

60. The method of any one of claims 39-59, wherein after the substituting of the one or more nucleotides in the DRACH motif, the method of codon optimization further comprises modulating polynucleotide GC content, polynucleotide secondary structure, mRNA motifs, ribosomal binding sites, and/or rates of translation.

61. The method of claim 60, wherein the polynucleotide GC content is adjusted to between 50% and 80%.

62. The method of claim 61, wherein the polynucleotide GC content is adjusted to between 65% and 75%.

63. The method of any one of claims 39-62, wherein the amino acid sequence of the protein or polypeptide shares at least 85% sequence identity to a wild-type amino acid sequence of the protein or polypeptide following codon optimization.

64. The method of anyone of claims 39-63, wherein the amino acid sequence of the protein or polypeptide shares 100% sequence identity to a wild-type amino acid sequence of the protein or polypeptide following codon optimization.

65. The method of any one of claims 39-64, wherein the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method.

66. The method of claim 65, wherein the number of DRACH motifs in the polynucleotide sequence is reduced by 100%, as compared to a corresponding polynucleotide sequence that has not been subjected to the method.

67. The method of any one of claims 39-66, wherein the method results in increased mRNA transcript stability or half-life.

68. The method of claim 67, wherein the increased mRNA transcript stability or half-life is assessed by an increase in concentration or relative abundance as compared to a corresponding polynucleotide sequence that has not been subjected to the method.

69. The method of claim 68, wherein the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

70. The method of claim 67, wherein the increased mRNA transcript stability or half-life is assessed by reduced rates of decay as detected by one or more pulse-chase methodologies.

71. The method of any one of claims 39-70, wherein reducing the number of DRACH motifs in the polynucleotide sequence reduces the number of N(6)-methyladenosine (m6A) modifications in an mRNA transcript as compared to a corresponding polynucleotide sequence that has not been subjected to the method.

72. The method of any one of claims 39-71, wherein the method results in increased protein expression or stability as compared to the protein encoded by a corresponding polynucleotide sequence that has not been subjected to the method.

73. The method of any one of claims 65-72, wherein the corresponding polynucleotide sequence is a wild-type polynucleotide sequence.

74. The method of claim 72 or 73, wherein the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a corresponding polynucleotide sequence that has not been subjected to the method.

75. The method of any one of claims 72-74, wherein the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a corresponding polynucleotide sequence that has not been subjected to the method.

76. The method of any one of claims 72-75, wherein the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

77. The method of any one of claims 39-41 and 45-76, wherein the polynucleotide is delivered to the host cell by contacting the host cell with a vehicle comprising the polynucleotide.

78. The method of any one of claims 42-76, wherein the polynucleotide is delivered to the subject by administering to the subject a vehicle comprising the polynucleotide.

79. The method of claim 77 or 78, wherein the vehicle is selected from the group consisting of a lipid nanoparticle, a liposome, a viral genome, and a viral vector.

80. The method of claim 79, wherein the viral vector is an adeno-associated virus (AAV), an adenovirus, a retrovirus, or a lentivirus.

81. The method of any one of claims 77-80, wherein the viral vector is an AAV.

82. The method of claim 81, wherein the AAV is pseudotyped.

83. The method of claim 81 or 82, wherein the AAV comprises an inverted terminal repeat (ITR) sequence and a capsid protein derived from different serotypes.

84. The method of any one of claims 39-41 and 45-83, wherein the delivery to the host cell occurs in vivo, in vitro, or ex vivo.

85. The method of any one of claims 42-83, wherein the delivery to the subject occurs by way of (i) in vivo administration of the polynucleotide to the subject or (ii) ex vivo expression of the polynucleotide in a host cell, followed by administration of the host cell to the subject.

86. The method of any one of claims 1-85, wherein the method is used to treat a subject that has or is at risk of having a disease characterized by low expression or activity of a protein.

87. A method of treating a subject who has or is at risk of developing a disease, the method comprising (i) substituting one or more nucleotides in a DRACH motif within a polynucleotide sequence encoding a polypeptide associated with the disease with an equivalent quantity of alternative nucleotides that eliminate the DRACH motif, wherein the substituting does not alter the amino acid sequence of the encoded polypeptide, and (ii) administering the polynucleotide sequence to the subject.

88. A method of treating a subject who has or is at risk of developing a disease, the method comprising administering a polynucleotide sequence encoding a polypeptide associated with the disease to the subject, wherein prior to the administering, one or more nucleotides in a DRACH motif within the polynucleotide sequence has been substituted with an equivalent quantity of alternative nucleotides that eliminate the DRACH motif, wherein the substituting does not alter the amino acid sequence of the encoded polypeptide.

89. The method of claim 87 or 88, wherein the substituting is performed in a DRACH motif selected from the group consisting of AGACA, AGACT, GAACC, GAACT, GGACA, GGACT, TGACT, AAACA, AAACC, AAACT, AGACC, GAACA, GGACC, TAACA, TAACC, TAACT, TGACA, and TGACC.

90. The method of claim 89, wherein the substituting is performed in a DRACH motif selected from the group consisting of AGACA, AGACT, GAACC, GAACT, GGACA, GGACT, and TGACT.

91. The method of any one of claims 87-90, wherein the substituting of the one or more nucleotides in the DRACH motif is performed using one or more codon usage guidelines selected from the group consisting of:
(a) never using the codon GAC to encode aspartic acid when eliminating the DRACH motif;
(b) never using the codon GAA to encode glutamic acid when eliminating the DRACH motif;
(c) never using the codon GGA to encode glycine when eliminating the DRACH motif;
(d) never using the codon AAA to encode lysine when eliminating the DRACH motif;
(e) never using the codon AAC to encode asparagine when eliminating the DRACH motif; and/or
(f) always using the codon ACG to encode threonine when eliminating the DRACH motif.

92. The method of any one of claims 87-91, wherein prior to the administering, the polynucleotide sequence has been codon optimized by a method comprising implementing, across the full polynucleotide sequence, one or more codon usage guidelines selected from the group consisting of:
(a) never using the codon GAC to encode aspartic acid across the full polynucleotide sequence;
(b) never using the codon GAA to encode glutamic acid across the full polynucleotide sequence;
(c) never using the codon GGA to encode glycine across the full polynucleotide sequence;
(d) never using the codon AAA to encode lysine across the full polynucleotide sequence;
(e) never using the codon AAC to encode asparagine across the full polynucleotide sequence; and/or
(f) always using the codon ACG to encode threonine across the full polynucleotide sequence.

93. The method of any one of claims 87-92, wherein prior to the substituting of the one or more nucleotides in the DRACH motif, the polynucleotide sequence has been codon optimized by substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

94. The method of any one of claims 87-93, wherein prior to the substituting of the one or more nucleotides in the DRACH motif, the polynucleotide sequence has been codon optimized by substituting one or more codons in the polynucleotide sequence with an equivalent quantity of synonymous codons that occur naturally at a higher frequency in a target organism relative to the frequency with which the one or more unmodified codons occur in the target organism.

95. The method of claim 93 or 94, wherein the target organism is a mammal, optionally wherein the mammal is a human.

96. The method of any one of claims 87-95, wherein prior to the substituting of the one or more nucleotides in the DRACH motif, the polynucleotide sequence has been codon optimized by way of a codon optimization webtool, codon optimization software, quantum computing, a heuristic scoring method, a neural network, or a combination thereof.

97. The method of any one of claims 87-98, wherein prior to the substituting of the one or more nucleotides in the DRACH motif, the polynucleotide sequence has been codon optimized by modulating polynucleotide guanine-cytosine (GC) content, polynucleotide secondary structure, mRNA motifs, ribosomal binding sites, and/or rates of translation.

98. The method of claim 97, wherein the polynucleotide GC content is adjusted to between 50% and 80%.

99. The method of claim 98, wherein the polynucleotide GC content is adjusted to between 65% and 75%.

100. The method of any one of claims 87-99, wherein after the substituting of the one or more nucleotides in the DRACH motif, the method further comprises substituting one or more nucleotides in a codon with an equivalent quantity of alternative nucleotides that, together, comprise a synonymous codon that occurs naturally at a higher frequency in a target organism relative to the frequency with which the unmodified codon occurs in the target organism.

101. The method of any one of claims 87-100, wherein after the substituting of the one or more nucleotides in the DRACH motif, the method further comprises substituting one or more codons in the polynucleotide sequence with an equivalent quantity of synonymous codons that occur naturally at a higher frequency in a target organism relative to the frequency with which the one or more unmodified codons occur in the target organism.

102. The method of claim 100 or 101, wherein the target organism is a mammal, optionally wherein the mammal is a human.

103. The method of any one of claims 87-102, wherein after the substituting of the one or more nucleotides in the DRACH motif, the method further comprises codon optimizing by way of a codon optimization webtool, codon optimization software, quantum computing, a heuristic scoring method, a neural network, or a combination thereof.

104. The method of any one of claims 87-103, wherein after the substituting of the one or more nucleotides in the DRACH motif, the method further comprises modulating the polynucleotide GC content, polynucleotide secondary structure, mRNA motifs, ribosomal binding sites, and/or rates of translation.

105. The method of claim 104, wherein the polynucleotide GC content is adjusted to between 50% and 80%.

106. The method of claim 105, wherein the polynucleotide GC content is adjusted to between 65% and 75%.

107. The method of any one of claims 87-106, wherein the amino acid sequence of the protein or polypeptide shares at least 85% sequence identity to a wild-type amino acid sequence of the protein or polypeptide following codon optimization.

108. The method of anyone of claims 87-107, wherein the amino acid sequence of the protein or polypeptide shares 100% sequence identity to a wild-type amino acid sequence of the protein or polypeptide following codon optimization.

109. The method of any one of claims 87-108, wherein the number of DRACH motifs in the polynucleotide sequence is reduced by at least 90%, at least 80%, at least 70%, at least 60%, at least 50%, at least 40%, at least 30%, at least 20%, or at least 10%, as compared to a corresponding polynucleotide sequence that has not been subjected to the method.

110. The method of claim 109, wherein the number of DRACH motifs in the polynucleotide sequence is reduced by 100%, as compared to a corresponding polynucleotide sequence that has not been subjected to the method.

111. The method of any one of claims 87-110, wherein the method results in increased mRNA transcript stability or half-life.

112. The method of claim 111, wherein the increased mRNA transcript stability or half-life is assessed by an increase in concentration or relative abundance as compared to a reference.

113. The method of claim 112, wherein the increase in mRNA transcript concentration or relative abundance as compared to the mRNA transcript of the native polynucleotide sequence is measured by RT-PCR, fluorimetry, colorimetry, spectrophotometric analyses, flow cytometry, in situ hybridization methods, Northern blot analyses, or an array using one or more hybridizing oligonucleotides or antibodies against the mRNA transcript of interest.

114. The method of claim 87-113, wherein reducing the number of DRACH motifs in the polynucleotide sequence reduces the number of N(6)-methyladenosine (m6A) modifications in an mRNA transcript as compared to a corresponding polynucleotide sequence that has not been subjected to the method.

115. The method of any one of claims 87-114, wherein the method results in increased protein expression or stability as compared to a reference.

116. The method of any one of claims 109-115, wherein the corresponding polynucleotide sequence is a wild-type polynucleotide sequence.

117. The method of any one of claims 112-116, wherein the reference is a sample obtained from a subject that has not received treatment or a sample obtained from the subject prior to treatment.

118. The method of any one of claims 115-117, wherein the protein expression or stability is increased by at least 10%, by at least 20%, by at least 30%, by at least 40%, by at least 50%, by at least 60%, by at least 70%, by at least 80%, by at least 90%, by at least 100%, or more than 100% as compared to a reference.

119. The method of any one of claims 115-118, wherein the protein expression or stability is increased by about 5-fold, by about 6-fold, by about 7-fold, by about 8-fold, by about 9-fold, by about 10-fold, by about 15-fold, by about 20-fold, by about 25-fold, by about 30-fold, by about 35-fold, by about 40-fold, by about 45-fold, by about 50-fold, or greater than 50-fold as compared to a reference.

120. The method of any one of claims 115-119, wherein the increase in protein expression is measured by mass spectrometry, Western blot analysis, enzyme-linked immunosorbent assay (ELISA), immunoprecipitation methods, flow cytometry, fluorimetry, colorimetry, spectrophotometric analyses, or an array using aptamers or antibodies against the protein of interest.

121. The method of any one of claims 87-120, wherein the polynucleotide is administered to the subject by contacting the subject with a vehicle comprising the polynucleotide.

122. The method of claim 121, wherein the vehicle is selected from a lipid nanoparticle, a liposome, a viral genome, and a viral vector.

123. The method of claim 122, wherein the viral vector is an adeno-associated virus (AAV), an adenovirus, a retrovirus, a lentivirus, or a double-stranded DNA virus.

124. The method of claim 122 or 123, wherein the viral vector is an AAV.

125. The method of claim 124, wherein the AAV is pseudotyped.

126. The method of claim 124 or 125, wherein the AAV comprises an inverted terminal repeat (ITR) sequence and a capsid protein derived from different serotypes.

127. The method of any one of claims 87-126, wherein the administration occurs by directly providing the subject, in vivo, with the polynucleotide or by expressing the polynucleotide in a host cell ex vivo and subsequently administering the host cell to the subject.

128. The method of any one of claims 87-127, wherein the delivery is performed by intramuscular, subcutaneous, intradermal, intravenous, intraperitoneal, intraarterial, intraventricular, intranasal, intraorbital, intracranial, or intraosseous administration.

129. The method of any one of claims 87-128, wherein the treatment is a monotherapy.

130. The method of any one of claims 87-128, wherein the method is combined with the administration of one or more additional therapeutic agents.

131. A kit comprising a package insert that instructs a user to perform a method of codon optimization that comprises modifying a polypeptide-encoding polynucleotide sequence by substituting one or more nucleotides in a DRACH motif in the polynucleotide sequence without altering the amino acid sequence of the encoded polypeptide, thereby eliminating the one or more DRACH motifs.

132. A kit comprising a package insert that instructs a user to perform the method of any one of claims 1-37 and 39-130.

133. The kit of claim 131 or 132, wherein the kit comprises a device for administering the polynucleotide sequence to a subject.

134. The kit of any one of claims 131-133, wherein the kit comprises one or more binding molecules to detect the expression of an mRNA transcript encoding the polypeptide or the expression or activity of the polypeptide.


SEQUENCE LISTING (11 sequences)

SEQ ID NO: 1 - DNA, 696 bp, synthetic construct
  source 1..696: mol_type = other DNA
atggcccagagcaagcacggcctgaccaaggagatgaccatgaagtaccggatggagggctgcgtggacggccacaagttcgtgatcaccggcgagggcatcggctaccccttcaagggcaagcaggccatcaacctgtgcgtggtggagggcggccccctgcccttcgccgaggacatcctgagcgccgccttcatgtacggcaaccgggtgttcaccgagtacccccaggacatcgtggactacttcaagaacagctgccccgccggctacacctgggaccggagcttcctgttcgaggacggcgccgtgtgcatctgcaacgccgacatcaccgtgagcgtggaggagaactgcatgtaccacgagagcaagttctacggcgtgaacttccccgccgacggccccgtgatgaagaagatgaccgacaactgggagcccagctgcgagaagatcatccccgtgcccaagcagggcatcctgaagggcgacgtgagcatgtacctgctgctgaaggacggcggccggctgcggtgccagttcgacaccgtgtacaaggccaagagcgtgccccggaagatgcccgactggcacttcatccagcacaagctgacccgggaggaccggagcgacgccaagaaccagaagtggcacctgaccgagcacgccatcgccagcggcagcgccctgccctga

SEQ ID NO: 2 - DNA, 696 bp, synthetic construct
  source 1..696: mol_type = other DNA
atggcccagagcaagcacggcctgacgaaggagatgacgatgaagtaccggatggagggctgcgtggatggccacaagttcgtgatcacgggcgagggcatcggctaccccttcaagggcaagcaggccatcaatctgtgcgtggtggagggcggccccctgcccttcgccgaggatatcctgagcgccgccttcatgtacggcaatcgggtgttcacggagtacccccaggatatcgtggattacttcaagaatagctgccccgccggctacacgtgggatcggagcttcctgttcgaggatggcgccgtgtgcatctgcaatgccgatatcacggtgagcgtggaggagaattgcatgtaccacgagagcaagttctacggcgtgaatttccccgccgatggccccgtgatgaagaagatgacggataattgggagcccagctgcgagaagatcatccccgtgcccaagcagggcatcctgaagggcgatgtgagcatgtacctgctgctgaaggatggcggccggctgcggtgccagttcgatacggtgtacaaggccaagagcgtgccccggaagatgcccgattggcacttcatccagcacaagctgacgcgggaggatcggagcgatgccaagaatcagaagtggcacctgacggagcacgccatcgccagcggcagcgccctgccctga

SEQ ID NO: 3 - DNA, 696 bp, synthetic construct
  source 1..696: mol_type = other DNA
atggcccagtcaaaacatggactcactaaagagatgaccatgaaatacagaatggaggggtgcgtcgatggtcacaaattcgtcatcaccggcgaggggattggatatccattcaagggaaagcaggccattaacctgtgcgtcgtggaaggaggaccactgcctttcgcagaagatatcctcagtgccgcattcatgtatggaaacagggtcttcactgagtacccacaggatattgttgactattttaaaaattcttgcccagccggctatacatgggaccgctccttcctctttgaagacggagcggtgtgtatctgcaacgcagatatcacagtgtccgttgaggaaaactgcatgtatcacgaaagcaagttctatggcgtgaatttccctgcggacgggcccgttatgaagaagatgacagacaattgggagcccagttgcgagaaaatcattccagtgcccaagcaggggattctcaagggcgacgtaagcatgtacctgctgctaaaggatggaggtcgcttacgctgccagtttgacaccgtctataaagccaagagcgtcccccggaagatgccggactggcattttatccagcacaagctgacccgtgaggacaggtctgatgccaaaaatcagaagtggcacttaactgagcatgctatcgcaagcggatcagcactcccgtga

SEQ ID NO: 4 - DNA, 696 bp, synthetic construct
  source 1..696: mol_type = other DNA
atggcccagtcaaagcatggcctcacgaaggagatgacgatgaagtacagaatggaggggtgcgtcgatggtcacaagttcgtcatcacgggcgaggggattgggtatccattcaagggcaagcaggccattaatctgtgcgtcgtggagggcggcccactgcctttcgcagaggatatcctcagtgccgcattcatgtatggcaatagggtcttcacggagtacccacaggatattgttgattattttaagaattcttgcccagccggctatacgtgggatcgctccttcctctttgaggatggggcggtgtgtatctgcaatgcagatatcacggtgtccgttgaggagaattgcatgtatcacgagagcaagttctatggcgtgaatttccctgcggatgggcccgttatgaagaagatgacggataattgggagcccagttgcgagaagatcattccagtgcccaagcaggggattctcaagggcgatgtaagcatgtacctgctgctaaaggatggcggtcgcttacgctgccagtttgatacggtctataaggccaagagcgtcccccggaagatgccggattggcattttatccagcacaagctgacgcgtgaggataggtctgatgccaagaatcagaagtggcacttaacggagcatgctatcgcaagcggttcagcactcccgtga

SEQ ID NO: 5 - DNA, 921 bp, synthetic construct
  source 1..921: mol_type = other DNA
atgatccccgcaaaggacatggcgaaggtaatgattgtgatgctggcaatctgctttctgactaagtcagacgggaagtcagttaagaagcgccacggcgaaggcacctttaccagtgatgttagctcgtatctggaggaacaggccgccaaagagttcatcgcctggctcgtgaaggggggaggcggaggaggtggatctgggggcggtgggtccggtggtggcggctccgcggaatcgaaatacggccctccttgtccaccatgccccgctcctgaagccgcaggaggaccctctgtctttctcttcccacctaagcctaaagatactcttatgataagtcgaacccctgaggtgacatgcgtggtggtcgatgtttcacaggaagaccctgaggtccagttcaactggtatgttgatggggtagaggtacacaatgccaaaacaaagcctagagaggaacagttcaatagcacatatcgcgtggtttccgtgctgacagtgctgcaccaggactggctcaacggaaaggagtacaagtgtaaggttagcaacaaaggccttccaagttcgatagagaagacaatctctaaagctaagggacagcctagagagccacaggtttacactctgccccccagtcaggaggaaatgaccaaaaaccaggtcagcctgacttgtctggtgaagggattctatccatcagatattgccgtcgagtgggaaagcaatgggcagcctgagaataattacaaaaccacaccccccgtgctggatagcgacggcagctttttcctatattcccgacttaccgttgacaagtcccgctggcaggaaggcaatgtgttttcttgcagtgtgatgcacgaggcattgcataatcactatacccagaaaagcctgagcctgtccctggggtga

SEQ ID NO: 6 - DNA, 921 bp, synthetic construct
  source 1..921: mol_type = other DNA
atgatccccgcaaaggatatggcgaaggtaatgattgtgatgctggcaatctgctttctgacgaagtcagacgggaagtcagttaagaagcgccacggcgaaggcacctttaccagtgatgttagctcgtatctggaggagcaggccgccaaagagttcatcgcctggctcgtgaaggggggaggcggaggaggtggatctgggggcggtgggtccggtggtggcggctccgcggaatcgaaatacggccctccttgtccaccatgccccgctcctgaagccgcaggaggcccctctgtctttctcttcccacctaagcctaaagatactcttatgataagtcgaacgcctgaggtgacgtgcgtggtggtcgatgtttcacaggaggatcctgaggtccagttcaactggtatgttgatggggtagaggtacacaatgccaagacgaagcctagagaggagcagttcaatagcacatatcgcgtggtttccgtgctgacggtgctgcaccaggattggctcaacggaaaggagtacaagtgtaaggttagcaacaaaggccttccaagttcgatagagaagacgatctctaaagctaagggccagcctagagagccacaggtttacactctgccccccagtcaggaggaaatgacgaagaatcaggtcagcctgacgtgtctggtgaagggattctatccatcagatattgccgtcgagtgggaaagcaatgggcagcctgagaataattacaagacgacaccccccgtgctggatagcgacggcagctttttcctatattcccgacttaccgttgataagtcccgctggcaggaaggcaatgtgttttcttgcagtgtgatgcacgaggcattgcataatcactatacccagaaaagcctgagcctgtccctggggtga

SEQ ID NO: 7 - DNA, 921 bp, synthetic construct
  source 1..921: mol_type = other DNA
atgatccccgccaaggacatggccaaggtgatgatcgtgatgctggccatctgcttcctgaccaagtccgacggcaagtccgtgaagaagcgccacggcgagggcaccttcaccagcgacgtgtctagctacctggaggagcaggccgccaaggagttcatcgcctggctggtgaagggcggcggcggcggcggcggcagcggcggcggcggcagcggcggcggcggcagcgccgagagcaagtacggccccccctgccccccctgccccgcccccgaggccgccggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtttctaacaagggcctgcccagcagcatcgagaagaccatcagcaaggccaagggccagcccagggagccccaggtgtacaccctgccccccagccaggaggagatgaccaagaaccaagttagcctgacctgcctggtgaagggcttctaccccagcgacatcgccgtggagtgggagagcaacggccagcccgagaacaactacaagaccaccccccccgtgctggacagcgacggcagcttcttcctgtacagccgactgaccgtggacaagagcaggtggcaggagggcaacgtgttcagctgcagcgtgatgcacgaggccctgcacaaccactacacccagaagagcctgagcctgagcctgggctga

SEQ ID NO: 8 - DNA, 921 bp, synthetic construct
  source 1..921: mol_type = other DNA
atgatccccgccaaggatatggccaaggtgatgatcgtgatgctggccatctgcttcctgacgaagtccgacggcaagtccgtgaagaagcgccacggcgagggcaccttcaccagcgacgtgtctagctacctggaggagcaggccgccaaggagttcatcgcctggctggtgaagggcggcggcggcggcggcggcagcggcggcggcggcagcggcggcggcggcagcgccgagagcaagtacggccccccctgccccccctgccccgcccccgaggccgccggcggccccagcgtgttcctgttcccccccaagcccaaggataccctgatgatcagcaggacgcccgaggtgacgtgcgtggtggtggacgtgagccaggaggatcccgaggtgcagttcaactggtacgtggacggcgtggaggtgcacaacgccaagacgaagcccagggaggagcagttcaacagcacctacagggtggtgagcgtgctgacggtgctgcaccaggattggctgaacggcaaggagtacaagtgcaaggtttctaataagggcctgcccagcagcatcgagaagacgatcagcaaggccaagggccagcccagggagccccaggtgtacaccctgccccccagccaggaggagatgacgaagaatcaagttagcctgacgtgcctggtgaagggcttctaccccagcgacatcgccgtggagtgggagagcaacggccagcccgagaataattacaagacgaccccccccgtgctggatagcgacggcagcttcttcctgtacagccgactgacggtggataagagcaggtggcaggagggcaacgtgttcagctgcagcgtgatgcacgaggccctgcacaaccactacacccagaagagcctgagcctgagcctgggctga

SEQ ID NO: 9 - protein, 306 aa, synthetic construct
MIPAKDMAKVMIVMLAICFLTKSDGKSVKKRHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 306 aa, synthetic construct; identical to SEQ ID NO: 9

SEQ ID NO: 11 - DNA, 921 bp, Homo sapiens
  source 1..921: mol_type = genomic DNA
atgatacctgcaaaagacatggctaaagttatgattgtcatgttggcaatttgttttcttacaaaatcggatgggaaatctgtaaagaagcggcatggagaagggacctttaccagtgatgtaagttcttatttggaagaacaagctgccaaggaattcattgcttggctggtgaaaggcggaggaggggggggggggtcaggcggagggggctccggaggaggaggatctgcagagtccaaatatggtcccccatgcccaccatgcccagcacctgaggctgcagggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggttga